SEQUENCE LISTING (214 sequences)

SEQ ID NO: 1 - protein, 13 aa, Artificial Sequence
  Synthetic Polypeptide
MAAAPSGCPDPTS

SEQ ID NO: 2 - protein, 13 aa, Artificial Sequence
  Synthetic Polypeptide
MAASVERGFIFLV

SEQ ID NO: 3 - protein, 24 aa, Artificial Sequence
  Synthetic Polypeptide
MAGGFMLMIVVMKLMAPKMEETPA

SEQ ID NO: 4 - protein, 12 aa, Artificial Sequence
  Synthetic Polypeptide
MAHTFPLNKTSR

SEQ ID NO: 5 - protein, 18 aa, Artificial Sequence
  Synthetic Polypeptide
MAHYSQIPSRPHGWPPSD

SEQ ID NO: 6 - protein, 16 aa, Artificial Sequence
  Synthetic Polypeptide
MAIAVVYPKTTIIPPK

SEQ ID NO: 7 - protein, 27 aa, Artificial Sequence
  Synthetic Polypeptide
MAIMIINDEYWLVVLVMVHCPESMLLK

SEQ ID NO: 8 - protein, 15 aa, Artificial Sequence
  Synthetic Polypeptide
MAPPLPPLEPTVKLT

SEQ ID NO: 9 - protein, 16 aa, Artificial Sequence
  Synthetic Polypeptide
MAQTMPMYPNGSFFPE

SEQ ID NO: 10 - protein, 23 aa, Artificial Sequence
  Synthetic Polypeptide
MATKKNFMEKGTRAGDMGSKPHS

SEQ ID NO: 11 - protein, 22 aa, Artificial Sequence
  Synthetic Polypeptide
MAVVDGWVNTWVVPKSASPWKN

SEQ ID NO: 12 - protein, 29 aa, Artificial Sequence
  Synthetic Polypeptide
MAYEGCCYSCKQEDNADVSGFWVEKWSVM

SEQ ID NO: 13 - protein, 21 aa, Artificial Sequence
  Synthetic Polypeptide
MCSCVYVYSYCKYMVCSHDKP

SEQ ID NO: 14 - protein, 10 aa, Artificial Sequence
  Synthetic Polypeptide
MDGCALLAVL

SEQ ID NO: 15 - protein, 12 aa, Artificial Sequence
  Synthetic Polypeptide
MDMVPQGKDEKL

SEQ ID NO: 16 - protein, 12 aa, Artificial Sequence
  Synthetic Polypeptide
MDSWVVGVNEWG

SEQ ID NO: 17 - protein, 28 aa, Artificial Sequence
  Synthetic Polypeptide
MDWSNWVWGVQLYVWDFLGSGCWAWTLS

SEQ ID NO: 18 - protein, 21 aa, Artificial Sequence
  Synthetic Polypeptide
MEAMGLAWNQLWGVRFLPFLS

SEQ ID NO: 19 - protein, 17 aa, Artificial Sequence
  Synthetic Polypeptide
METDLDYSGLNSDHVGL

SEQ ID NO: 20 - protein, 16 aa, Artificial Sequence
  Synthetic Polypeptide
MFDQENVVTSTESSPS

SEQ ID NO: 21 - protein, 29 aa, Artificial Sequence
  Synthetic Polypeptide
MFEELINVWVWVYMSQWEFYDGPCNEQCY

SEQ ID NO: 22 - protein, 30 aa, Artificial Sequence
  Synthetic Polypeptide
MFGSEFMYHSENSMMDHVTNNATGMNIMEK

SEQ ID NO: 23 - protein, 18 aa, Artificial Sequence
  Synthetic Polypeptide
MFHLRYHNHRYPHRRQSI

SEQ ID NO: 24 - protein, 11 aa, Artificial Sequence
  Synthetic Polypeptide
MFKLSFYFYVV

SEQ ID NO: 25 - protein, 10 aa, Artificial Sequence
  Synthetic Polypeptide
MFLVGWWGMV

SEQ ID NO: 26 - protein, 12 aa, Artificial Sequence
  Synthetic Polypeptide
MGACQGGSDESN

SEQ ID NO: 27 - protein, 13 aa, Artificial Sequence
  Synthetic Polypeptide
MGCDDNVVDVVVT

SEQ ID NO: 28 - protein, 25 aa, Artificial Sequence
  Synthetic Polypeptide
MGCSGSSVSQCYRVHTPQTKMPNAW

SEQ ID NO: 29 - protein, 9 aa, Artificial Sequence
  Synthetic Polypeptide
MGDCAVCDA

SEQ ID NO: 30 - protein, 19 aa, Artificial Sequence
  Synthetic Polypeptide
MGEGLEENPTNPITKPTLN

SEQ ID NO: 31 - protein, 12 aa, Artificial Sequence
  Synthetic Polypeptide
MGEVGLLWLGRI

SEQ ID NO: 32 - protein, 19 aa, Artificial Sequence
  Synthetic Polypeptide
MGGEWGMGVWTWGCFLVWM

SEQ ID NO: 33 - protein, 10 aa, Artificial Sequence
  Synthetic Polypeptide
MGGGVLLGLQ

SEQ ID NO: 34 - protein, 27 aa, Artificial Sequence
  Synthetic Polypeptide
MGGLGWGWPGGWVWGGGFMVEERWWEL

SEQ ID NO: 35 - protein, 13 aa, Artificial Sequence
  Synthetic Polypeptide
MGGRWGLGWWRAG

SEQ ID NO: 36 - protein, 19 aa, Artificial Sequence
  Synthetic Polypeptide
MGLAAVCVCWVGWAGVVLM

SEQ ID NO: 37 - protein, 21 aa, Artificial Sequence
  Synthetic Polypeptide
MGLLEWLLCWHLLGRIINWWA

SEQ ID NO: 38 - protein, 10 aa, Artificial Sequence
  Synthetic Polypeptide
MGLWLVCWVL

SEQ ID NO: 39 - protein, 14 aa, Artificial Sequence
  Synthetic Polypeptide
MGNIIRAWCGEGCL

SEQ ID NO: 40 - protein, 14 aa, Artificial Sequence
  Synthetic Polypeptide
MGSPPPAGSKKVVL

SEQ ID NO: 41 - protein, 13 aa, Artificial Sequence
  Synthetic Polypeptide
MGSSRINPDPSPS

SEQ ID NO: 42 - protein, 21 aa, Artificial Sequence
  Synthetic Polypeptide
MGSSRLAVLCPPLHGQVNFTG

SEQ ID NO: 43 - protein, 15 aa, Artificial Sequence
  Synthetic Polypeptide
MGVGMEWGLLLRRGR

SEQ ID NO: 44 - protein, 10 aa, Artificial Sequence
  Synthetic Polypeptide
MGVKFFTLST

SEQ ID NO: 45 - protein, 10 aa, Artificial Sequence
  Synthetic Polypeptide
MHGELPWVVN

SEQ ID NO: 46 - protein, 10 aa, Artificial Sequence
  Synthetic Polypeptide
MHHMKHPIIC

SEQ ID NO: 47 - protein, 16 aa, Artificial Sequence
  Synthetic Polypeptide
MHKHYYNKHPHHYNLP

SEQ ID NO: 48 - protein, 20 aa, Artificial Sequence
  Synthetic Polypeptide
MHKRLSPIYYSHRYLPDKRL

SEQ ID NO: 49 - protein, 16 aa, Artificial Sequence
  Synthetic Polypeptide
MHLVFSSGGYARDSIA

SEQ ID NO: 50 - protein, 17 aa, Artificial Sequence
  Synthetic Polypeptide
MHQSCKPEMKTFFQGQI

SEQ ID NO: 51 - protein, 11 aa, Artificial Sequence
  Synthetic Polypeptide
MIEMGASFCHV

SEQ ID NO: 52 - protein, 13 aa, Artificial Sequence
  Synthetic Polypeptide
MITVPAHKGMAMA

SEQ ID NO: 53 - protein, 24 aa, Artificial Sequence
  Synthetic Polypeptide
MIYVSSDVGDWWKGGWGVWWVVHG

SEQ ID NO: 54 - protein, 10 aa, Artificial Sequence
  Synthetic Polypeptide
MKFEIWLGWC

SEQ ID NO: 55 - protein, 14 aa, Artificial Sequence
  Synthetic Polypeptide
MKHRQVLWVLSCGS

SEQ ID NO: 56 - protein, 10 aa, Artificial Sequence
  Synthetic Polypeptide
MKNHRCISTT

SEQ ID NO: 57 - protein, 23 aa, Artificial Sequence
  Synthetic Polypeptide
MKPFTVHSTLQSNPFSSPWMTPL

SEQ ID NO: 58 - protein, 10 aa, Artificial Sequence
  Synthetic Polypeptide
MKPMSPMRLY

SEQ ID NO: 59 - protein, 23 aa, Artificial Sequence
  Synthetic Polypeptide
MKSPSTLTTQSKTPSAYFSSFSP

SEQ ID NO: 60 - protein, 16 aa, Artificial Sequence
  Synthetic Polypeptide
MLEIVMGMETYHMSNA

SEQ ID NO: 61 - protein, 15 aa, Artificial Sequence
  Synthetic Polypeptide
MLFMRGNAMSGAPII

SEQ ID NO: 62 - protein, 10 aa, Artificial Sequence
  Synthetic Polypeptide
MLGVVGWCRL

SEQ ID NO: 63 - protein, 29 aa, Artificial Sequence
  Synthetic Polypeptide
MLGWVVGSFFMGGVWVGRSGIGGMLFEFM

SEQ ID NO: 64 - protein, 26 aa, Artificial Sequence
  Synthetic Polypeptide
MLHNNPNPNTNYLPNWKQNTQMGLSL

SEQ ID NO: 65 - protein, 13 aa, Artificial Sequence
  Synthetic Polypeptide
MLKPETSSDSPSA

SEQ ID NO: 66 - protein, 17 aa, Artificial Sequence
  Synthetic Polypeptide
MLLGWRFQLGAWVGGLQ

SEQ ID NO: 67 - protein, 17 aa, Artificial Sequence
  Synthetic Polypeptide
MLLMWWVSEPHCVVVNM

SEQ ID NO: 68 - protein, 18 aa, Artificial Sequence
  Synthetic Polypeptide
MLLNSHRCSMSKDNHHSP

SEQ ID NO: 69 - protein, 13 aa, Artificial Sequence
  Synthetic Polypeptide
MLMVEWWLGWLHM

SEQ ID NO: 70 - protein, 15 aa, Artificial Sequence
  Synthetic Polypeptide
MLNWSNNLTNGTSYP

SEQ ID NO: 71 - protein, 18 aa, Artificial Sequence
  Synthetic Polypeptide
MLPLHIYHNTMGLTHPPH

SEQ ID NO: 72 - protein, 14 aa, Artificial Sequence
  Synthetic Polypeptide
MLQGQAHLSILFSI

SEQ ID NO: 73 - protein, 20 aa, Artificial Sequence
  Synthetic Polypeptide
MLRSMLWNLWSKPQFHAHRP

SEQ ID NO: 74 - protein, 26 aa, Artificial Sequence
  Synthetic Polypeptide
MLSPKPQQLNQQNCSPEHYEPQLKTQ

SEQ ID NO: 75 - protein, 22 aa, Artificial Sequence
  Synthetic Polypeptide
MLTPGLVNFVPATAVTRLTQVN

SEQ ID NO: 76 - protein, 26 aa, Artificial Sequence
  Synthetic Polypeptide
MLTSKYSNQPSTITHQLQLQSHPSPT

SEQ ID NO: 77 - protein, 26 aa, Artificial Sequence
  Synthetic Polypeptide
MLVPFDRGDLEGELTGTGMLACVILL

SEQ ID NO: 78 - protein, 13 aa, Artificial Sequence
  Synthetic Polypeptide
MMAHLGKKPVSGG

SEQ ID NO: 79 - protein, 13 aa, Artificial Sequence
  Synthetic Polypeptide
MMCLRFRWCGLWS

SEQ ID NO: 80 - protein, 20 aa, Artificial Sequence
  Synthetic Polypeptide
MMDPEHMNSMALKKAWVQMC

SEQ ID NO: 81 - protein, 14 aa, Artificial Sequence
  Synthetic Polypeptide
MMFPIRLHNSPIRP

SEQ ID NO: 82 - protein, 20 aa, Artificial Sequence
  Synthetic Polypeptide
MMGITMKKIITNAWAVTMTL

SEQ ID NO: 83 - protein, 14 aa, Artificial Sequence
  Synthetic Polypeptide
MMGMWLVGHYVLSC

SEQ ID NO: 84 - protein, 14 aa, Artificial Sequence
  Synthetic Polypeptide
MMKGKMKWKVKNRV

SEQ ID NO: 85 - protein, 18 aa, Artificial Sequence
  Synthetic Polypeptide
MMKLRLTPWRLPDPPNHH

SEQ ID NO: 86 - protein, 12 aa, Artificial Sequence
  Synthetic Polypeptide
MMLSWDDIIYGG

SEQ ID NO: 87 - protein, 11 aa, Artificial Sequence
  Synthetic Polypeptide
MMMFVYSAMKN

SEQ ID NO: 88 - protein, 14 aa, Artificial Sequence
  Synthetic Polypeptide
MMMTIECLHSHFPH

SEQ ID NO: 89 - protein, 10 aa, Artificial Sequence
  Synthetic Polypeptide
MMNSDSIIPS

SEQ ID NO: 90 - protein, 24 aa, Artificial Sequence
  Synthetic Polypeptide
MMSVWKVAVQTFNCYYYVLQALIN

SEQ ID NO: 91 - protein, 16 aa, Artificial Sequence
  Synthetic Polypeptide
MMTHPTTPLTINTKPP

SEQ ID NO: 92 - protein, 17 aa, Artificial Sequence
  Synthetic Polypeptide
MMTWSPWKPVATKNVEP

SEQ ID NO: 93 - protein, 24 aa, Artificial Sequence
  Synthetic Polypeptide
MMWKTIVVFQLQEHQWPQYAKLTP

SEQ ID NO: 94 - protein, 25 aa, Artificial Sequence
  Synthetic Polypeptide
MNCGGCLWGLVGSGYGVSSGVCVLG

SEQ ID NO: 95 - protein, 11 aa, Artificial Sequence
  Synthetic Polypeptide
MNHIMAKGHDG

SEQ ID NO: 96 - protein, 23 aa, Artificial Sequence
  Synthetic Polypeptide
MNHNQYYQSMLIINNHNSYSNKT

SEQ ID NO: 97 - protein, 10 aa, Artificial Sequence
  Synthetic Polypeptide
MNNSSSTVQP

SEQ ID NO: 98 - protein, 23 aa, Artificial Sequence
  Synthetic Polypeptide
MNPDQPHHLLLSLYTAIFSKPWW

SEQ ID NO: 99 - protein, 28 aa, Artificial Sequence
  Synthetic Polypeptide
MPFWVEVMMEVEIWCCEIVLGNSFSSQV

SEQ ID NO: 100 - protein, 11 aa, Artificial Sequence
  Synthetic Polypeptide
MPIIRRMSWSP

SEQ ID NO: 101 - protein, 26 aa, Artificial Sequence
  Synthetic Polypeptide
MPIVTIMSPSWLEVEKATIFLMSFCV

SEQ ID NO: 102 - protein, 20 aa, Artificial Sequence
  Synthetic Polypeptide
MPLRLEWVQWGVGGWPWVCC

SEQ ID NO: 103 - protein, 11 aa, Artificial Sequence
  Synthetic Polypeptide
MPPKDKIWNLV

SEQ ID NO: 104 - protein, 26 aa, Artificial Sequence
  Synthetic Polypeptide
MPPLCKIHCRIHLYYQSLPHNNIHVP

SEQ ID NO: 105 - protein, 13 aa, Artificial Sequence
  Synthetic Polypeptide
MPRKCWLYPSRTN

SEQ ID NO: 106 - protein, 11 aa, Artificial Sequence
  Synthetic Polypeptide
MPTNLPTLNST

SEQ ID NO: 107 - protein, 19 aa, Artificial Sequence
  Synthetic Polypeptide
MPTPILIFRSPWSLYSYPT

SEQ ID NO: 108 - protein, 15 aa, Artificial Sequence
  Synthetic Polypeptide
MQFTLATPKCYGPGA

SEQ ID NO: 109 - protein, 25 aa, Artificial Sequence
  Synthetic Polypeptide
MQNPPHSSPHSSPLPRYSYLSPLLY

SEQ ID NO: 110 - protein, 23 aa, Artificial Sequence
  Synthetic Polypeptide
MQPYRRYRFHPRLSMIYPTLQLM

SEQ ID NO: 111 - protein, 16 aa, Artificial Sequence
  Synthetic Polypeptide
MQRWFFMSLVVVVVRA

SEQ ID NO: 112 - protein, 20 aa, Artificial Sequence
  Synthetic Polypeptide
MQSGVLQSLAVTEIKYCNLL

SEQ ID NO: 113 - protein, 10 aa, Artificial Sequence
  Synthetic Polypeptide
MQTKSLQSPQ

SEQ ID NO: 114 - protein, 12 aa, Artificial Sequence
  Synthetic Polypeptide
MQWAILGLFVVG

SEQ ID NO: 115 - protein, 26 aa, Artificial Sequence
  Synthetic Polypeptide
MRLLAWGNTWWQLLWNEGLFFWLELE

SEQ ID NO: 116 - protein, 11 aa, Artificial Sequence
  Synthetic Polypeptide
MSGVRKEVSLL

SEQ ID NO: 117 - protein, 15 aa, Artificial Sequence
  Synthetic Polypeptide
MSKKIPATTMVLEWS

SEQ ID NO: 118 - protein, 11 aa, Artificial Sequence
  Synthetic Polypeptide
MSLSYLQDSTY

SEQ ID NO: 119 - protein, 12 aa, Artificial Sequence
  Synthetic Polypeptide
MSLTPHILPTMP

SEQ ID NO: 120 - protein, 11 aa, Artificial Sequence
  Synthetic Polypeptide
MSMPVLGWQWG

SEQ ID NO: 121 - protein, 10 aa, Artificial Sequence
  Synthetic Polypeptide
MSNMKTFSSA

SEQ ID NO: 122 - protein, 12 aa, Artificial Sequence
  Synthetic Polypeptide
MSNNMAFSTFKG

SEQ ID NO: 123 - protein, 25 aa, Artificial Sequence
  Synthetic Polypeptide
MSRNHSFCLNYMPIRFSLILFVVTH

SEQ ID NO: 124 - protein, 14 aa, Artificial Sequence
  Synthetic Polypeptide
MSSAWIIWFRLFSI

SEQ ID NO: 125 - protein, 12 aa, Artificial Sequence
  Synthetic Polypeptide
MSSDEFANTMPV

SEQ ID NO: 126 - protein, 15 aa, Artificial Sequence
  Synthetic Polypeptide
MSTHLPNYHTNLSYR

SEQ ID NO: 127 - protein, 21 aa, Artificial Sequence
  Synthetic Polypeptide
MSTINQQVIITLTVNPTQACS

SEQ ID NO: 128 - protein, 17 aa, Artificial Sequence
  Synthetic Polypeptide
MSWSNIEVVNPIVDMDS

SEQ ID NO: 129 - protein, 17 aa, Artificial Sequence
  Synthetic Polypeptide
MSYRAHTPKMLVMPFPY

SEQ ID NO: 130 - protein, 12 aa, Artificial Sequence
  Synthetic Polypeptide
MTAPVKLQGVWM

SEQ ID NO: 131 - protein, 15 aa, Artificial Sequence
  Synthetic Polypeptide
MTELLTPNWTNLSPY

SEQ ID NO: 132 - protein, 28 aa, Artificial Sequence
  Synthetic Polypeptide
MTKNFHQTPPPPLLATALKHISAKPQKQ

SEQ ID NO: 133 - protein, 13 aa, Artificial Sequence
  Synthetic Polypeptide
MTLGHNMIYLHTS

SEQ ID NO: 134 - protein, 10 aa, Artificial Sequence
  Synthetic Polypeptide
MTNIFGVWAR

SEQ ID NO: 135 - protein, 22 aa, Artificial Sequence
  Synthetic Polypeptide
MTNPENQNERKSVRFIHCPHNP

SEQ ID NO: 136 - protein, 13 aa, Artificial Sequence
  Synthetic Polypeptide
MTNTLNSIHPPLP

SEQ ID NO: 137 - protein, 22 aa, Artificial Sequence
  Synthetic Polypeptide
MTSLHNSFYSKDTSLRTPLMTP

SEQ ID NO: 138 - protein, 20 aa, Artificial Sequence
  Synthetic Polypeptide
MTSPKIQNNNTPDHTANNQY

SEQ ID NO: 139 - protein, 18 aa, Artificial Sequence
  Synthetic Polypeptide
MTWPTEQVTLGMTAQSYS

SEQ ID NO: 140 - protein, 21 aa, Artificial Sequence
  Synthetic Polypeptide
MTYSPEQSQLQYMHQQTMFNQ

SEQ ID NO: 141 - protein, 14 aa, Artificial Sequence
  Synthetic Polypeptide
MVEGWLLYLLVSMG

SEQ ID NO: 142 - protein, 12 aa, Artificial Sequence
  Synthetic Polypeptide
MVEVGYNVVRVV

SEQ ID NO: 143 - protein, 23 aa, Artificial Sequence
  Synthetic Polypeptide
MVGYLIPVWVLAIVCSDMLKPLS

SEQ ID NO: 144 - protein, 23 aa, Artificial Sequence
  Synthetic Polypeptide
MVIICFEVSLISHCWVVISRLLM

SEQ ID NO: 145 - protein, 11 aa, Artificial Sequence
  Synthetic Polypeptide
MVKPHLQNASI

SEQ ID NO: 146 - protein, 20 aa, Artificial Sequence
  Synthetic Polypeptide
MVLEFGLVGYFLLGGGSGWV

SEQ ID NO: 147 - protein, 10 aa, Artificial Sequence
  Synthetic Polypeptide
MVLFLLGMVG

SEQ ID NO: 148 - protein, 25 aa, Artificial Sequence
  Synthetic Polypeptide
MVLNLRVHRLRRTNLQLLHTSPIIP

SEQ ID NO: 149 - protein, 16 aa, Artificial Sequence
  Synthetic Polypeptide
MVLSSPPIFRMSCSLL

SEQ ID NO: 150 - protein, 10 aa, Artificial Sequence
  Synthetic Polypeptide
MVLWWFGGSG

SEQ ID NO: 151 - protein, 11 aa, Artificial Sequence
  Synthetic Polypeptide
MVNGRRTSVGV

SEQ ID NO: 152 - protein, 10 aa, Artificial Sequence
  Synthetic Polypeptide
MVRMLLVQWW

SEQ ID NO: 153 - protein, 10 aa, Artificial Sequence
  Synthetic Polypeptide
MVSMMTSMGM

SEQ ID NO: 154 - protein, 12 aa, Artificial Sequence
  Synthetic Polypeptide
MVSSGDWYSWCE

SEQ ID NO: 155 - protein, 19 aa, Artificial Sequence
  Synthetic Polypeptide
MVVCMVITFIWSCTKIFGA

SEQ ID NO: 156 - protein, 19 aa, Artificial Sequence
  Synthetic Polypeptide
MVVHWMSGVGLPWLWGVGV

SEQ ID NO: 157 - protein, 21 aa, Artificial Sequence
  Synthetic Polypeptide
MVVKGPLSEGGHPWGREGIWL

SEQ ID NO: 158 - protein, 14 aa, Artificial Sequence
  Synthetic Polypeptide
MWEEAGRMSEGCLG

SEQ ID NO: 159 - protein, 13 aa, Artificial Sequence
  Synthetic Polypeptide
MWGVSDMLVFLEV

SEQ ID NO: 160 - protein, 11 aa, Artificial Sequence
  Synthetic Polypeptide
MWLGVGLGWVG

SEQ ID NO: 161 - protein, 12 aa, Artificial Sequence
  Synthetic Polypeptide
MWLIEEYAMSDF

SEQ ID NO: 162 - protein, 10 aa, Artificial Sequence
  Synthetic Polypeptide
MWLVLWLWML

SEQ ID NO: 163 - protein, 11 aa, Artificial Sequence
  Synthetic Polypeptide
MWMPPTLPHIR

SEQ ID NO: 164 - protein, 11 aa, Artificial Sequence
  Synthetic Polypeptide
MWNDLLQCSEP

SEQ ID NO: 165 - protein, 10 aa, Artificial Sequence
  Synthetic Polypeptide
MWRVLGALWW

SEQ ID NO: 166 - protein, 19 aa, Artificial Sequence
  Synthetic Polypeptide
MWSHPSHHSTINITNKWLL

SEQ ID NO: 167 - protein, 30 aa, Artificial Sequence
  Synthetic Polypeptide
MWVIEEYAKILRSWVWFNPPQLPAMMDKIE

SEQ ID NO: 168 - protein, 10 aa, Artificial Sequence
  Synthetic Polypeptide
MYCNWDCSGE

SEQ ID NO: 169 - protein, 10 aa, Artificial Sequence
  Synthetic Polypeptide
MYLNGTCSAS

SEQ ID NO: 170 - protein, 10 aa, Artificial Sequence
  Synthetic Polypeptide
MYNYAKAPTL

SEQ ID NO: 171 - protein, 14 aa, Artificial Sequence
  Synthetic Polypeptide
MYRHLQQTLMKATK

SEQ ID NO: 172 - protein, 13 aa, Artificial Sequence
  Synthetic Polypeptide
MYTNKQCSTSNYY

SEQ ID NO: 173 - protein, 18 aa, Artificial Sequence
  Synthetic Polypeptide
MYTPTNNVQPVTTTNQRP

SEQ ID NO: 174 - protein, 16 aa, Artificial Sequence
  Synthetic Polypeptide
MYYSDGYWGVSWGMGV

SEQ ID NO: 175 - protein, 27 aa, Artificial Sequence
  Synthetic Polypeptide
MYEMHSGCWWVSQYLGGTQICFPMKEQ

SEQ ID NO: 176 - protein, 10 aa, Artificial Sequence
  Synthetic Polypeptide
MKNIEAPLAW

SEQ ID NO: 177 - protein, 16 aa, Artificial Sequence
  Synthetic Polypeptide
MRMGASIGSTTRLSTS

SEQ ID NO: 178 - protein, 23 aa, Artificial Sequence
  Synthetic Polypeptide
MVQTVSISWASEMLVLVSFVVSV

SEQ ID NO: 179 - protein, 12 aa, Artificial Sequence
  Synthetic Polypeptide
MSSSMMGEVASC

SEQ ID NO: 180 - protein, 17 aa, Artificial Sequence
  Synthetic Polypeptide
MVVGSWCWSLSWVVGMR

SEQ ID NO: 181 - protein, 14 aa, Artificial Sequence
  Synthetic Polypeptide
MCHWAGGASNTGDA

SEQ ID NO: 182 - protein, 11 aa, Artificial Sequence
  Synthetic Polypeptide
MFAVSMMWSLE

SEQ ID NO: 183 - protein, 11 aa, Artificial Sequence
  Synthetic Polypeptide
MMKVDATMDFT

SEQ ID NO: 184 - protein, 10 aa, Artificial Sequence
  Synthetic Polypeptide
MASAKGCSSP

SEQ ID NO: 185 - protein, 12 aa, Artificial Sequence
  Synthetic Polypeptide
MLVMLEVMFLVN

SEQ ID NO: 186 - protein, 24 aa, Artificial Sequence
  Synthetic Polypeptide
MIINDEYWLVVLVMVHCPESMLLK

SEQ ID NO: 187 - protein, 24 aa, Artificial Sequence
  Synthetic Polypeptide
AYEGCCYSCKQEDNADVSGFWVEK

SEQ ID NO: 188 - protein, 24 aa, Artificial Sequence
  Synthetic Polypeptide
DWSNWVWGVQLYVWDFLGSGCWAW

SEQ ID NO: 189 - protein, 24 aa, Artificial Sequence
  Synthetic Polypeptide
FEELINVWVWVYMSQWEFYDGPCN

SEQ ID NO: 190 - protein, 24 aa, Artificial Sequence
  Synthetic Polypeptide
MYHSENSMMDHVTNNATGMNIMEK

SEQ ID NO: 191 - protein, 24 aa, Artificial Sequence
  Synthetic Polypeptide
LGWGWPGGWVWGGGFMVEERWWEL

SEQ ID NO: 192 - protein, 24 aa, Artificial Sequence
  Synthetic Polypeptide
LGWVVGSFFMGGVWVGRSGIGGML

SEQ ID NO: 193 - protein, 24 aa, Artificial Sequence
  Synthetic Polypeptide
LHNNPNPNTNYLPNWKQNTQMGLS

SEQ ID NO: 194 - protein, 24 aa, Artificial Sequence
  Synthetic Polypeptide
LSPKPQQLNQQNCSPEHYEPQLKT

SEQ ID NO: 195 - protein, 24 aa, Artificial Sequence
  Synthetic Polypeptide
LTSKYSNQPSTITHQLQLQSHPSP

SEQ ID NO: 196 - protein, 24 aa, Artificial Sequence
  Synthetic Polypeptide
LVPFDRGDLEGELTGTGMLACVIL

SEQ ID NO: 197 - protein, 24 aa, Artificial Sequence
  Synthetic Polypeptide
WVEVMMEVEIWCCEIVLGNSFSSQ

SEQ ID NO: 198 - protein, 24 aa, Artificial Sequence
  Synthetic Polypeptide
PIVTIMSPSWLEVEKATIFLMSFC

SEQ ID NO: 199 - protein, 24 aa, Artificial Sequence
  Synthetic Polypeptide
PLCKIHCRIHLYYQSLPHNNIHVP

SEQ ID NO: 200 - protein, 24 aa, Artificial Sequence
  Synthetic Polypeptide
RLLAWGNTWWQLLWNEGLFFWLEL

SEQ ID NO: 201 - protein, 24 aa, Artificial Sequence
  Synthetic Polypeptide
TKNFHQTPPPPLLATALKHISAKP

SEQ ID NO: 202 - protein, 24 aa, Artificial Sequence
  Synthetic Polypeptide
MWVIEEYAKILRSWVWFNPPQLPA

SEQ ID NO: 203 - protein, 9 aa, Artificial Sequence
  Synthetic Polypeptide
KNIEAPLAW

SEQ ID NO: 204 - protein, 15 aa, Artificial Sequence
  Synthetic Polypeptide
RMGASIGSTTRLSTS

SEQ ID NO: 205 - protein, 22 aa, Artificial Sequence
  Synthetic Polypeptide
VQTVSISWASEMLVLVSFVVSV

SEQ ID NO: 206 - protein, 11 aa, Artificial Sequence
  Synthetic Polypeptide
SSSMMGEVASC

SEQ ID NO: 207 - protein, 16 aa, Artificial Sequence
  Synthetic Polypeptide
VVGSWCWSLSWVVGMR

SEQ ID NO: 208 - protein, 13 aa, Artificial Sequence
  Synthetic Polypeptide
CHWAGGASNTGDA

SEQ ID NO: 209 - protein, 10 aa, Artificial Sequence
  Synthetic Polypeptide
FAVSMMWSLE

SEQ ID NO: 210 - protein, 10 aa, Artificial Sequence
  Synthetic Polypeptide
MKVDATMDFT

SEQ ID NO: 211 - protein, 11 aa, Artificial Sequence
  Synthetic Polypeptide
YARAAARQARA

SEQ ID NO: 212 - protein, 7 aa, Artificial Sequence
  Synthetic Polypeptide
YGRKKRR

SEQ ID NO: 213 - protein, 11 aa, Artificial Sequence
  Synthetic Polypeptide
YGRKKRRQRRR

SEQ ID NO: 214 - protein, 5 aa, Artificial Sequence
  Synthetic Polypeptide
RRQRR

(19) United States
(12) Patent Application Publication
(10) Pub. No.: US 2022/0332774 A1
(43) Pub. Date: Oct. 20, 2022

(54) THERAPEUTIC MITOCHONDRIAL PEPTIDES

(71) Applicant: COHBAR, INC., Menlo Park, CA (US)

(72) Inventor: Kenneth Cundy, Atherton, CA (US)

(21) Appl. No.: 17/634,416

(22) PCT Filed: Aug. 12, 2020
(86) PCT No.: PCT/US20/45980

§ 371 Date: Feb. 10, 2022

Related U.S. Application Data
(60) Provisional application No. 62/885,394, filed on Aug. 12, 2019.

(51) Int. Cl. C07K 14/47; A61K 38/14; A61P 3/04; A61P 3/10; A61K 35/00; A61K 47/64

(52) U.S. Cl. CPC C07K 14/47; A61K 38/14; A61P 3/04; A61P 3/10; A61K 35/00; A61K 47/64

(57) ABSTRACT

The disclosures herein relate to the fields of cell biology and the modulation of cellular mechanisms controlling cell viability, cell proliferation, and metabolic processes. More specifically disclosed herein are peptides effective to modulate cellular mechanisms controlling cell viability, cell proliferation, and metabolic processes, including cell signaling associated with aberrant cellular proliferation and malignancy. Also disclosed herein are peptides effective in modulating cellular mechanisms controlling cell viability, treating metabolic diseases, and as cytoprotective agents.

### CROSS REFERENCES TO RELATED APPLICATIONS

This application claims priority benefit of U.S. Provisional Application No. 62/885,394, filed on Aug. 12, 2019, which is hereby incorporated by reference in its entirety and for all purposes as if fully set forth herein.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “53073A_Seqlisting.txt”, which was created on Aug. 12, 2020 and is 56,463 bytes in size. The subject matter of the Sequence Listing is incorporated herein in its entirety by reference.

### TECHNICAL FIELD

This disclosure relates to the fields of cell biology and the modulation of cell viability and metabolic processes. More specifically disclosed are peptides effective to modulate cell signaling associated with aberrant cellular proliferation and malignancy. Also disclosed are peptides effective in modulating cell viability, treating metabolic diseases and as cytoprotective agents.

### BACKGROUND

The control of cellular behavior is not clearly understood. Dysregulation of cellular metabolic pathways can lead to imbalance in energy homeostasis and may result in a wide range of metabolic disorders, including but not limited to obesity, diabetes, hypertension, arteriosclerosis, high cholesterol, hyperlipidemia, fatty liver disease, non-alcoholic steatohepatitis (NASH), and other diseases. The precise cellular mechanisms regulating cellular apoptosis are not completely known. Dysregulation of apoptosis has been implicated in a number of human diseases. An inappropriate suppression of apoptosis in a cell may lead to the uncontrolled propagation of that cell, potentially favoring the development of cancer. In contrast, a failure to control the extent of apoptotic cell death may lead to degeneration of specific tissues and cell-types, such as occurs in neurodegeneration, autoimmune disorders, and other diseases.

There is a need for more effective therapies modulating cellular mechanisms that control the activity of cells, including for example cell metabolism, cell proliferation, and cell viability. More specifically, there remains a great need for more effective treatments that can address a wide range of metabolic disorders by safely regulating metabolic pathways. There is a need for more effective therapies modulating cellular mechanisms including those that induce or suppress apoptosis in cells and/or tissues of individuals suffering from disorders characterized by inappropriate cell proliferation or inappropriate cell death.

Mitochondria, central to metabolic processes in eukaryotic cells, are involved in numerous cellular processes, including among others energy production, ATP synthesis, reactive oxygen species (ROS) generation, programmed cell death, signaling, cellular differentiation, and control of the cell cycle and cell growth. A small number of mitochondrial DNA-derived signaling peptides have been identified to date with diverse structures and widely differing biological properties. Despite this effort, the natural occurrence and function of the vast majority of theoretical mitochondrial DNA-derived peptide sequences remains undefined, while their potential biological activity as exogenous peptides is completely unknown and cannot be predicted from their structure. The inventors have identified therapeutically useful isolated peptides with unexpected properties based on mitochondrial DNA and conceived novel analogs and derivatives with improved properties.

### SUMMARY

Disclosed are peptides comprising amino acid sequences of SEQ ID NO: 1-210, that exhibit activity in modulating cellular mechanisms. Also disclosed are peptides comprising amino acid sequences SEQ ID NO: 1-210, analogs and derivatives thereof.

The present disclosure moreover includes pharmaceutical compositions comprising amino acid sequences SEQ ID NO: 1-210, analogs and derivatives thereof described herein and a pharmaceutically acceptable excipient, as well as a method of treating or preventing a disease or medical condition (e.g., diabetes, obesity, non-alcoholic steatohepatitis (NASH), cancer, metabolic diseases, neurodegenerative diseases, cardiovascular diseases) in a patient. The method comprises administering to the patient a presently disclosed peptide, derivative or analog, optionally formulated into a pharmaceutical composition, in an amount effective to treat an appropriate disease or medical condition.

### DETAILED DESCRIPTION

In one aspect, peptides that therapeutically modulate cellular mechanisms are disclosed.

In one embodiment, a peptide of any one or more of the amino acid sequences set forth in any one of SEQ ID NO: 1-210 are disclosed.

In some embodiments, a peptide is represented by the peptides listed in Table 1.

TABLE 1

| SEQ ID NO: | SEQUENCE |
|---|---|
| 1 | MAAAPSGCPDPTS |
| 2 | MAASVERGFIFLV |
| 3 | MAGGFMLMIVVMKLMAPKMEETPA |
| 4 | MAHTFPLNKTSR |
| 5 | MAHYSQIPSRPHGWPPSD |
| 6 | MAIAVVYPKTTIIPPK |
| 7 | MAIMIINDEYWLVVLVMVHCPESMLLK |
| 8 | MAPPLPPLEPTVKLT |
| 9 | MAQTMPMYPNGSFFPE |
| 10 | MATKKNFMEKGTRAGDMGSKPHS |
| 11 | MAVVDGWVNTWVVPKSASPWKN |
| 12 | MAYEGCCYSCKQEDNADVSGFWVEKWSVM |
| 13 | MCSCVYVYSYCKYMVCSHDKP |
| 14 | MDGCALLAVL |
| 15 | MDMVPQGKDEKL |
| 16 | MDSWVVGVNEWG |
| 17 | MDWSNWVWGVQLYVWDFLGSGCWAWTLS |
| 18 | MEAMGLAWNQLWGVRFLPFLS |
| 19 | METDLDYSGLNSDHVGL |
| 20 | MFDQENVVTSTESSPS |
| 21 | MFEELINVWVWVYMSQWEFYDGPCNEQCY |
| 22 | MFGSEFMYHSENSMMDHVTNNATGMNIMEK |
| 23 | MFHLRYHNHRYPHRRQSI |
| 24 | MFKLSFYFYVV |
| 25 | MFLVGWWGMV |
| 26 | MGACQGGSDESN |
| 27 | MGCDDNVVDVVVT |
| 28 | MGCSGSSVSQCYRVHTPQTKMPNAW |
| 29 | MGDCAVCDA |
| 30 | MGEGLEENPTNPITKPTLN |
| 31 | MGEVGLLWLGRI |
| 32 | MGGEWGMGVWTWGCFLVWM |
| 33 | MGGGVLLGLQ |
| 34 | MGGLGWGWPGGWVWGGGFMVEERWWEL |
| 35 | MGGRWGLGWWRAG |
| 36 | MGLAAVCVCWVGWAGVVLM |
| 37 | MGLLEWLLCWHLLGRIINWWA |
| 38 | MGLWLVCWVL |
| 39 | MGNIIRAWCGEGCL |
| 40 | MGSPPPAGSKKVVL |
| 41 | MGSSRINPDPSPS |
| 42 | MGSSRLAVLCPPLHGQVNFTG |
| 43 | MGVGMEWGLLLRRGR |
| 44 | MGVKFFTLST |
| 45 | MHGELPWVVN |
| 46 | MHHMKHPIIC |
| 47 | MHKHYYNKHPHHYNLP |
| 48 | MHKRLSPIYYSHRYLPDKRL |
| 49 | MHLVFSSGGYARDSIA |
| 50 | MHQSCKPEMKTFFQGQI |
| 51 | MIEMGASFCHV |
| 52 | MITVPAHKGMAMA |
| 53 | MIYVSSDVGDWWKGGWGVWWVVHG |
| 54 | MKFEIWLGWC |
| 55 | MKHRQVLWVLSCGS |
| 56 | MKNHRCISTT |
| 57 | MKPFTVHSTLQSNPFSSPWMTPL |
| 58 | MKPMSPMRLY |
| 59 | MKSPSTLTTQSKTPSAYFSSFSP |
| 60 | MLEIVMGMETYHMSNA |
| 61 | MLFMRGNAMSGAPII |
| 62 | MLGVVGWCRL |
| 63 | MLGWVVGSFFMGGVWVGRSGIGGMLFEFM |
| 64 | MLHNNPNPNTNYLPNWKQNTQMGLSL |
| 65 | MLKPETSSDSPSA |
| 66 | MLLGWRFQLGAWVGGLQ |
| 67 | MLLMWWVSEPHCVVVNM |
| 68 | MLLNSHRCSMSKDNHHSP |
| 69 | MLMVEWWLGWLHM |
| 70 | MLNWSNNLTNGTSYP |
| 71 | MLPLHIYHNTMGLTHPPH |
| 72 | MLQGQAHLSILFSI |
| 73 | MLRSMLWNLWSKPQFHAHRP |
| 74 | MLSPKPQQLNQQNCSPEHYEPQLKTQ |
| 75 | MLTPGLVNFVPATAVTRLTQVN |
| 76 | MLTSKYSNQPSTITHQLQLQSHPSPT |
| 77 | MLVPFDRGDLEGELTGTGMLACVILL |
| 78 | MMAHLGKKPVSGG |
| 79 | MMCLRFRWCGLWS |
| 80 | MMDPEHMNSMALKKAWVQMC |
| 81 | MMFPIRLHNSPIRP |
| 82 | MMGITMKKIITNAWAVTMTL |
| 83 | MMGMWLVGHYVLSC |
| 84 | MMKGKMKWKVKNRV |
| 85 | MMKLRLTPWRLPDPPNHH |
| 86 | MMLSWDDIIYGG |
| 87 | MMMFVYSAMKN |
| 88 | MMMTIECLHSHFPH |
| 89 | MMNSDSIIPS |
| 90 | MMSVWKVAVQTFNCYYYVLQALIN |
| 91 | MMTHPTTPLTINTKPP |
| 92 | MMTWSPWKPVATKNVEP |
| 93 | MMWKTIVVFQLQEHQWPQYAKLTP |
| 94 | MNCGGCLWGLVGSGYGVSSGVCVLG |
| 95 | MNHIMAKGHDG |
| 96 | MNHNQYYQSMLIINNHNSYSNKT |
| 97 | MNNSSSTVQP |
| 98 | MNPDQPHHLLLSLYTAIFSKPWW |
| 99 | MPFWVEVMMEVEIWCCEIVLGNSFSSQV |
| 100 | MPIIRRMSWSP |
| 101 | MPIVTIMSPSWLEVEKATIFLMSFCV |
| 102 | MPLRLEWVQWGVGGWPWVCC |
| 103 | MPPKDKIWNLV |
| 104 | MPPLCKIHCRIHLYYQSLPHNNIHVP |
| 105 | MPRKCWLYPSRTN |
| 106 | MPTNLPTLNST |
| 107 | MPTPILIFRSPWSLYSYPT |
| 108 | MQFTLATPKCYGPGA |
| 109 | MQNPPHSSPHSSPLPRYSYLSPLLY |
| 110 | MQPYRRYRFHPRLSMIYPTLQLM |
| 111 | MQRWFFMSLVVVVVRA |
| 112 | MQSGVLQSLAVTEIKYCNLL |
| 113 | MQTKSLQSPQ |
| 114 | MQWAILGLFVVG |
| 115 | MRLLAWGNTWWQLLWNEGLFFWLELE |
| 116 | MSGVRKEVSLL |
| 117 | MSKKIPATTMVLEWS |
| 118 | MSLSYLQDSTY |
| 119 | MSLTPHILPTMP |
| 120 | MSMPVLGWQWG |
| 121 | MSNMKTFSSA |
| 122 | MSNNMAFSTFKG |
| 123 | MSRNHSFCLNYMPIRFSLILFVVTH |
| 124 | MSSAWIIWFRLFSI |
| 125 | MSSDEFANTMPV |
| 126 | MSTHLPNYHTNLSYR |
| 127 | MSTINQQVIITLTVNPTQACS |
| 128 | MSWSNIEVVNPIVDMDS |
| 129 | MSYRAHTPKMLVMPFPY |
| 130 | MTAPVKLQGVWM |
| 131 | MTELLTPNWTNLSPY |
| 132 | MTKNFHQTPPPPLLATALKHISAKPQKQ |
| 133 | MTLGHNMIYLHTS |
| 134 | MTNIFGVWAR |
| 135 | MTNPENQNERKSVRFIHCPHNP |
| 136 | MTNTLNSIHPPLP |
| 137 | MTSLHNSFYSKDTSLRTPLMTP |
| 138 | MTSPKIQNNNTPDHTANNQY |
| 139 | MTWPTEQVTLGMTAQSYS |
| 140 | MTYSPEQSQLQYMHQQTMFNQ |
| 141 | MVEGWLLYLLVSMG |
| 142 | MVEVGYNVVRVV |
| 143 | MVGYLIPVWVLAIVCSDMLKPLS |
| 144 | MVIICFEVSLISHCWVVISRLLM |
| 145 | MVKPHLQNASI |
| 146 | MVLEFGLVGYFLLGGGSGWV |
| 147 | MVLFLLGMVG |
| 148 | MVLNLRVHRLRRTNLQLLHTSPIIP |
| 149 | MVLSSPPIFRMSCSLL |
| 150 | MVLWWFGGSG |
| 151 | MVNGRRTSVGV |
| 152 | MVRMLLVQWW |
| 153 | MVSMMTSMGM |
| 154 | MVSSGDWYSWCE |
| 155 | MVVCMVITFIWSCTKIFGA |
| 156 | MVVHWMSGVGLPWLWGVGV |
| 157 | MVVKGPLSEGGHPWGREGIWL |
| 158 | MWEEAGRMSEGCLG |
| 159 | MWGVSDMLVFLEV |
| 160 | MWLGVGLGWVG |
| 161 | MWLIEEYAMSDF |
| 162 | MWLVLWLWML |
| 163 | MWMPPTLPHIR |
| 164 | MWNDLLQCSEP |
| 165 | MWRVLGALWW |
| 166 | MWSHPSHHSTINITNKWLL |
| 167 | MWVIEEYAKILRSWVWFNPPQLPAMMDKIE |
| 168 | MYCNWDCSGE |
| 169 | MYLNGTCSAS |
| 170 | MYNYAKAPTL |
| 171 | MYRHLQQTLMKATK |
| 172 | MYTNKQCSTSNYY |
| 173 | MYTPTNNVQPVTTTNQRP |
| 174 | MYYSDGYWGVSWGMGV |
| 175 | MYEMHSGCWWVSQYLGGTQICFPMKEQ |
| 176 | MKNIEAPLAW |
| 177 | MRMGASIGSTTRLSTS |
| 178 | MVQTVSISWASEMLVLVSFVVSV |
| 179 | MSSSMMGEVASC |
| 180 | MVVGSWCWSLSWVVGMR |
| 181 | MCHWAGGASNTGDA |
| 182 | MFAVSMMWSLE |
| 183 | MMKVDATMDFT |
| 184 | MASAKGCSSP |
| 185 | MLVMLEVMFLVN |
| 186 | MIINDEYWLVVLVMVHCPESMLLK |
| 187 | AYEGCCYSCKQEDNADVSGFWVEK |
| 188 | DWSNWVWGVQLYVWDFLGSGCWAW |
| 189 | FEELINVWVWVYMSQWEFYDGPCN |
| 190 | MYHSENSMMDHVTNNATGMNIMEK |
| 191 | LGWGWPGGWVWGGGFMVEERWWEL |
| 192 | LGWVVGSFFMGGVWVGRSGIGGML |
| 193 | LHNNPNPNTNYLPNWKQNTQMGLS |
| 194 | LSPKPQQLNQQNCSPEHYEPQLKT |
| 195 | LTSKYSNQPSTITHQLQLQSHPSP |
| 196 | LVPFDRGDLEGELTGTGMLACVIL |
| 197 | WVEVMMEVEIWCCEIVLGNSFSSQ |
| 198 | PIVTIMSPSWLEVEKATIFLMSFC |
| 199 | PLCKIHCRIHLYYQSLPHNNIHVP |
| 200 | RLLAWGNTWWQLLWNEGLFFWLEL |
| 201 | TKNFHQTPPPPLLATALKHISAKP |
| 202 | MWVIEEYAKILRSWVWFNPPQLPA |
| 203 | KNIEAPLAW |
| 204 | RMGASIGSTTRLSTS |
| 205 | VQTVSISWASEMLVLVSFVVSV |
| 206 | SSSMMGEVASC |
| 207 | VVGSWCWSLSWVVGMR |
| 208 | CHWAGGASNTGDA |
| 209 | FAVSMMWSLE |
| 210 | MKVDATMDFT |

In some embodiments, peptides disclosed herein comprise a sequence having at least 70% sequence identity to any one of amino acid sequences SEQ ID NO: 1-210. In certain embodiments, the % identity is selected from, e.g., at least 75%, at least 80%, at least 85%, at least 90%, or at least 95%, or more sequence identity to a given sequence. In certain embodiments, the % identity is in the range of, e.g., about 65% to about 70%, about 70% to about 80%, about 80% to about 85%, about 85% to about 90%, or about 90% to about 95%; between about 70% and about 80%, between about 80% and about 90% and between about 90% and about 99% sequence identity.

In some embodiments, a peptide is in a modified form of any one of amino acid sequences SEQ ID NO: 1-210, comprising up to 4 amino acid modifications relative to any one of amino acid sequences SEQ ID NO: 1-210. In some embodiments, a peptide is in a modified form of any one of amino acid sequences SEQ ID NO: 1-210 comprising up to 3 amino acid modifications relative to any one of amino acid sequences SEQ ID NO: 1-210. In some embodiments, a peptide comprises up to 2 amino acid modifications relative any one of amino acid sequences SEQ ID NO: 1-210. In some embodiments, a peptide comprises 1 amino acid modification relative any one of amino acid sequences SEQ ID NO: 1-210.

In certain embodiments, the peptide comprises a sequence having at least 70% sequence identity to any one of amino acid sequences SEQ ID NO: 1-210. In certain embodiments, the % identity is selected from, e.g., at least 75%, at least 80%, at least 85%, at least 90%, or at least 95%, or more sequence identity to a given sequence. In certain embodiments, the % identity is in the range of, e.g., about 65% to about 70%, about 70% to about 80%, about 80% to about 85%, about 85% to about 90%, or about 90% to about 95%; between about 70% and about 80%, between about 80% and about 90% and between about 90% and about 99% sequence identity, but does not comprise the sequence set forth in any of SEQ ID NO: 1-210.

Peptides of the disclosure include peptides that have been modified in any way and for any reason, for example, to: (1) reduce susceptibility to proteolysis, (2) alter binding affinities, and (3) confer or modify other physicochemical or functional properties. For example, single or multiple amino acid substitutions (e.g., equivalent, conservative or non-conservative substitutions, deletions or additions) may be made in a sequence.

A conservative amino acid substitution refers to the substitution in a peptide of an amino acid with a functionally similar amino acid having similar properties, e.g., size, charge, hydrophobicity, hydrophilicity, and/or aromaticity. The following six groups each contain amino acids that are conservative substitutions for one another are found in Table 2.

TABLE 2
i. Alanine (A), Serine (S), and Threonine (T)
ii. Aspartic acid (D) and Glutamic acid (E)
iii. Asparagine (N) and Glutamine (Q)
iv. Arginine (R) and Lysine (K)
v. Isoleucine (I), Leucine (L), Methionine (M), and Valine (V)
vi. Phenylalanine (F), Tyrosine (Y), and Tryptophan (W)

Additionally, within the meaning of the term “equivalent amino acid substitution” as applied herein, one amino acid may be substituted for another, in one embodiment, within the groups of amino acids indicated herein below:
1. Amino acids with polar side chains (Asp, Glu, Lys, Arg, His, Asn, Gln, Ser, Thr, Tyr, and
Cys,)
2. Amino acids with small nonpolar or slightly polar residues (Ala, Ser, Thr, Pro, Gly);
3. Amino acids with non-polar side chains (Gly, Ala, Val, Leu, Ile, Phe, Trp, Pro, and Met)
4. Amino acids with large, aliphatic, nonpolar residues (Met, Leu, Ile, Val, Cys, Norleucine (Nle), homocysteine)
5. Amino acids with aliphatic side chains (Gly, Ala Val, Leu, Ile)
6. Amino acids with cyclic side chains (Phe, Tyr, Trp, His, Pro)
7. Amino acids with aromatic side chains (Phe, Tyr, Trp)
8. Amino acids with acidic side chains (Asp, Glu)
9. Amino acids with basic side chains (Lys, Arg, His)
10. Amino acids with amide side chains (Asn, Gln)
11. Amino acids with hydroxy side chains (Ser, Thr)
12. Amino acids with sulphur-containing side chains (Cys, Met),
13. Neutral, weakly hydrophobic amino acids (Pro, Ala, Gly, Ser, Thr)
14. Hydrophilic, acidic amino acids (Gln, Asn, Glu, Asp), and
15. Hydrophobic amino acids (Leu, Ile, Val).

In some embodiments, the amino acid substitution is not a conservative amino acid substitution, e.g., is a non-conservative amino acid substitution. This class generally includes corresponding D-amino acids, homo-amino acids, N-alkyl amino acids, beta amino acids and other unnatural amino acids. The non-conservative amino acid substitutions still fall within the descriptions identified for the equivalent amino acid substitutions above [e.g. polar, nonpolar, etc.]. Examples of non-conservative amino acids are provided below.

Non limiting examples for alanine non-conservative amino acids are: D-alanine [Dala, (dA), a], N-Acetyl-3-(3,4-dimethoxyphenyl)-D-alanine, N-Me-D-Ala-OH, N-Me-Ala-OH, H-β-Ala-β-naphthalene, L-(−)-2-Amino-3-ureidopropionic acid, (R)-(+)-α-Allylalanine, (S)-(−)-α-Allylalanine, D-2-Aminobutyric acid, L-2-Aminobutyric acid, DL-2-Aminobutyric acid, 2-Aminoisobutyric acid, α-Aminoisobutyric acid, (S)-(+)-2-Amino-4-phenylbutyric acid ethyl ester, Benzyl α-aminoisobutyrate, Abu-OH, Aib-OH, β-(9-anthryl)-Ala-OH, β-(3-benzothienyl)-Ala-OH, β-(3-benzothienyl)-D-Ala-OH, Cha-OH, Cha-OMe, β-(2-furyl)-Ala-OH, β-(2-furyl)-D-Ala-OH, β-iodo-Ala-OBzl, β-iodo-D-Ala-OBzl, 3-iodo-D-Ala-OMe, β-iodo-Ala-OMe, 1-Nal-OH, D-1-Nal-OH, 2-Nal-OH, D-2-Nal-OH, (R)-3-(2-naphthyl)-β-Ala-OH, (S)-3-(2-naphthyl)-β-Ala-OH, β-phenyl-Phe-OH, 3-(2-pyridyl)-Ala-OH, 3-(3-pyridyl)-Ala-OH, 3-(3-pyridyl)-D-Ala-OH, (S)-3-(3-pyridyl)-β-Ala-OH, 3-(4-pyridyl)-Ala-OH, 3-(4-pyridyl)-D-Ala-OH, β-(2-quinolyl)-Ala-OH, 3-(2-quinolyl)-DL-Ala-OH, 3-(3-quinolyl)-DL-Ala-OH, 3-(2-quinoxalyl)-DL-Ala-OH, β-(4-thiazolyl)-Ala-OH, β-(2-thienyl)-Ala-OH, β-(2-thienyl)-D-Ala-OH, β-(3-thienyl)-Ala-OH, β-(3-thienyl)-D-Ala-OH, 3-Chloro-D-alanine methyl ester, N-[(4-Chlorophenyl)sulfonyl]-β-alanine, 3-Cyclohexyl-D-alanine, 3-Cyclopentyl-DL-alanine, (−)-3-(3,4-Dihydroxyphenyl)-2-methyl-L-alanine, 3,3-Diphenyl-D-alanine, 3,3-Diphenyl-L-alanine, N—[(S)-(+)-1-(Ethoxycarbonyl)-3-phenylpropyl]-L-alanine, N-[1-(S)-(+)-Ethoxycarbonyl-3-phenylpropyl]-L-alanyl carboxyanhydride, N-(3-fluorobenzyl)alanine, N-(3-Indolylacetyl)-L-alanine, Methyl (RS)-2-(aminomethyl)-3-phenylpropionate, 3-(2-Oxo-1,2-dihydro-4-quinolinyl)alanine, 3-(1-Pyrazolyl)-L-alanine, 3-(2-Pyridyl)-D-alanine, 3-(2-Pyridyl)-L-alanine, 3-(3-Pyridyl)-L-alanine, 3-(4-Pyridyl)-D-alanine, 3-(4-Pyridyl)-L-alanine, 3-(2-Quinolyl)-DL-alanine, 3-(4-Quinolyl)-DL-alanine, D-styrylalanine, L-styrylalanine, 3-(2-Thienyl)-L-alanine, 3-(2-Thienyl)-DL-alanine, 3-(2-Thienyl)-DL-alanine, 3,3,3-Trifluoro-DL-alanine, N-Methyl-L-alanine, 3-Ureidopropionic acid, Aib-OH, Cha-OH, Dehydro-Ala-OMe, dehydro-Ala-OH, D-2-Nal-OH, β-Ala-ONp, β-Homoala-OH, β-D-Homoala-OH, β-Alanine, β-Alanine ethyl ester, β-Alanine methyl ester, (S)-diphenyl-β-Homoala-OH, (R)-4-(4-pyridyl)-β-Homoala-OH, (S)-4-(4-pyridyl)-β-Homoala-OH, β-Ala-OH, (S)-diphenyl-β-Homoala-OH, L-β-Homoalanine, (R)-4-(3-pyridyl)-β-Homoala-OH, α-methyl-α-naphthylalanine [Manap], N-methyl-cyclohexylalanine [Nmchexa], cyclohexylalanine [Chexa], N-methyl-cyclopentylalanine [Nmcpen], cyclopentylalanine [Cpen], N-methyl-α-naphthylalanine [Nmanap], α-naphthylalanine [Anap], L-N-methylalanine [Nmala], D-N-methylalanine [Dnmala], α-methyl-cyclohexylalanine [Mchexa], α-methyl-cyclopentylalanine [Mcpen]. Each possibility represents a separate embodiment.

Non limiting examples for arginine non-conservative amino acids are: homoarginine (hArg), N-methyl arginine (NMeArg), citruline, 2-amino-3-guanidinopropionic acid, N-iminoethyl-L-ornithine, Nω-monomethyl-L-arginine, Nω-nitro-L-arginine, D-arginine, 2-amino-3-ureidopropionic acid, Nω,ω-dimethyl-L-arginine, Nω-Nitro-D-arginine, L-α-methylarginine [Marg], D-α-methylarginine [Dmarg], L-N-methylarginine [Nmarg], D-N-methylarginine [Dnmarg], β-Homoarg-OH, L-Homoarginine, N-(3-guanidinopropyl)glycine [Narg], and D-arginine [Darg, (dR), r]. Each possibility represents a separate embodiment.

Non limiting examples for asparagine non-conservative amino acids are: L-α-methylasparagine [Masn], D-α-methylasparagine [Dmasn], L-N-methylasparagine [Nmasn], D-N-methylasparagine [Dnmasn], N-(carbamylmethyl)glycine [Nasn] and D-asparagine [Dasn, (dN), n]. Each possibility represents a separate embodiment.

Non limiting examples for aspartic acid non-conservative amino acids are: L-α-methylaspartate [Masp], D-α-methylaspartate [Dmasp], L-N-methylaspartic acid [Nmasp], D-N-methylasparatate [Dnmasp], N-(carboxymethyl)glycine [Nasp] and D-aspartic acid [Dasp, (dD), d]. Each possibility represents a separate embodiment.

Non limiting examples for cysteine non-conservative amino acids are: L-Cysteic acid, L-Cysteinesulfinic acid, D-Ethionine, S-(2-Thiazolyl)-L-cysteine, DL-Homocysteine, L-Homocysteine, L-Homocystine, L-α-methylcysteine [Mcys], D-α-methylcysteine [Dmcys], L-N-methylcysteine [Nmcys], D-N-methylcysteine [Dnmcys], N-(thiomethyl)glycine [Ncys] and D-cysteine [Dcys, (dC), c]. Each possibility represents a separate embodiment.

Non limiting examples for glutamic acid non-conservative amino acids are: γ-Carboxy-DL-glutamic acid, 4-Fluoro-DL-glutamic acid, β-Glutamic acid, L-β-Homoglutamic acid, L-α-methylglutamate [Mglu], D-α-methyl glutamic acid [Dmglu], L-N-methylglutamic acid [Nmglu], D-N-methylglutamate [Dnmglu], N-(2-carboxyethyl)glycine [Nglu], and D-glutamic acid [Dglu, (dE), e]. Each possibility represents a separate embodiment.

Non limiting examples for glutamine non-conservative amino acids are: Cit-OH, D-Citrulline, Thio-L-citrulline, β-Gln-OH, L-β-Homoglutamine, L-α-methylglutamine [Mgln], D-α-methylglutamine [Dmgln], L-N-methylglutamine [Nmgln], D-N-methylglutamine [Dnmgln], N-(2-carbamylethyl)glycine [Ngln], and D-glutamine [Dgln, (dQ), q]. Each possibility represents a separate embodiment.

Non limiting examples for glycine non-conservative amino acids are: tBu-Gly-OH,D-Allylglycine, N-[Bis(methylthio)methylene]glycine methyl ester, Chg-OH, D-Chg-OH, D-cyclopropylglycine, L-cyclopropylglycine, (R)-4-fluorophenylglycine, (S)-4-fluorophenylglycine, iminodiacetic acid, (2-indanyl)-Gly-OH, (±)-α-phosphonoglycine trimethyl ester, D-propargylglycine, propargyl-Gly-OH, (R)-2-thienylglycine, (S)-2-thienylglycine, (R)-3-thienylglycine, (S)-3-thienylglycine, 2-(4-trifluoromethyl-phenyl)-DL-glycine, (2S,3R,4S)-α-(Carboxycyclopropyl)glycine, N-(Chloroacetyl)glycine ethyl ester, (S)-(+)-2-chlorophenylglycine methyl ester, N-(2-chlorophenyl)-N-(methylsulfonyl)glycine, D-α-Cyclohexylglycine, L-α-Cyclopropylglycine, Di-tert-butyl-iminodicarboxylate, Ethyl acetamidocyanoacetate, N-(2-fluorophenyl)-N-(methylsulfonyl) glycine, N-(4-fluorophenyl)-N-(methylsulfonyl)glycine, N-(2-Furfurylideneacetyl)glycine methyl ester, N-(2-Furoyl)glycine, N-(2-Hydroxyethyl)iminodiacetic acid, N-(4-Hydroxyphenyl)glycine, Iminodiacetic acid, N-Lauroylsarcosine sodium salt, L-α-Neopentylglycine, N-(Phosphonomethyl)glycine, D-Propargylglycine, L-C-Propargylglycine, Sarcosine, N,N-Dimethylglycine, N,N-Dimethylglycine ethyl ester, D-Chg-OH, α-Phosphonoglycine trimethyl ester, N-cyclobutylglycine [Ncbut], L-α-methylethylglycine [Metg], N-cycloheptylglycine [Nchep], L-α-methyl-1-butylglycine [Mtbug], N-methylglycine [Nmgly], L-N-methyl-ethylglycine [Nmetg], L-ethylglycine [Etg], L-N-methyl-t-butylglycine [Nmtbug], L-t-butylglycine [Tbug], N-cyclohexylglycine [Nchex], N-cyclodecylglycine [Ncdec], N-cyclododecylglycine [Ncdod], N-cyclooctylglycine [Ncoct], N-cyclopropylglycine [Ncpro], N-cycloundecylglycine [Ncund], N-(2-aminoethyl)glycine [Naeg], N—(N-(2,2-diphenylethyl) diphenylethyl)glycine [Nnbhm], N-(2,2-carbamylmethyl-glycine [Nbhm], N—(N-(3,3-diphenylpropyl) diphenylpropyl)glycine [Nnbhe] and N-(3,3-carbamylmethyl-glycine [Nbhe]. Each possibility represents a separate embodiment.

Non limiting examples for histidine non-conservative amino acids are: L-α-methylhistidine [Mhis], D-α-methylhistidine [Dmhis], L-N-methylhistidine [Nmhis], D-N-methylhistidine [Dnmhis], N-(imidazolylethyl)glycine [Nhis], and D-histidine [Dhis, (dH), h]. Each possibility represents a separate embodiment.

Non limiting examples for isoleucine non-conservative amino acids are: N-Methyl-L-isoleucine [Nmile], N-(3-Indolylacetyl)-L-isoleucine, allo-Ile-OH, D-allo-Isoleucine, L-β-Homoisoleucine, L-α-methylisoleucine [Mile], D-α-methylisoleucine [Dmile], D-N-methylisoleucine [Dnmile], N-(1-methylpropyl)glycine [Nile], and D-isoleucine [Dile, (dD), i]. Each possibility represents a separate embodiment.

Non limiting examples for leucine non-conservative amino acids are: D-leuine [Dleu, (dL), 1]. Cycloleucine, DL-leucine, N-Formyl-Leu-OH, D-tert-Leucine, L-tert-Leucine, DL-tert-Leucine, L-tert-Leucine methyl ester, 5,5,5-Trifluoro-DL-leucine, D-β-Leu-OH, L-β-Leucine, DL-β-Leucine, L-β-Homoleucine, DL-β-Homoleucine, L-N-methyl-leucine [Nmleu], D-N-methyl-leucine [Dnmleu], L-α-methyl-leucine [Mleu], D-α-methyl-leucine [Dmleu], N-(2-methylpropyl)glycine [Nleu], D-leucine [Dleu, 1], D-Norleucine, L-Norleucine, DL-Norleucine, L-N-methylnorleucine [Nmnle] and L-norleucine [Nle]. Each possibility represents a separate embodiment.

Non limiting examples for lysine non-conservative amino acids are: DL-5-Hydroxylysine, (5R)-5-Hydroxy-L-lysine, β-Lys-OH, L-β-Homolysine, L-α-methyl-lysine [Mlys], D-α-methyl-lysine [Dmlys], L-N-methyl-lysine [Nmlys], D-N-methyl-lysine [Dnmlys], N-(4-aminobutyl)glycine [Nlys], and D-lysine [Dlys, (dK), k]. Each possibility represents a separate embodiment.

Non limiting examples for methionine non-conservative amino acids are: L-β-Homomethionine, DL-β-Homomethionine, L-α-methylmethionine [Mmet], D-α-methylmethionine [Dmmet], L-N-methylmethionine [Nmmet], D-N-methylmethionine [Dnmmet], N-(2-methylthioethyl)glycine [Nmet], and D-methionine [Duret, (dM), m]. Each possibility represents a separate embodiment.

Non limiting examples for phenylalanine non-conservative amino acids are: N-Acetyl-2-fluoro-DL-phenylalanine, N-Acetyl-4-fluoro-DL-phenylalanine, 4-Amino-L-phenylalanine, 3-[3,4-bis(trifluoromethyl)phenyl]-L-alanine, Bpa-OH, D-Bpa-OH, 4-tert-butyl-Phe-OH, 4-tert-butyl-D-Phe-OH, 4-(amino)-L-phenylalanine, rac-β²-homophenylalanine, 2-methoxy-L-phenylalanine, (S)-4-methoxy-β-Phe-OH, 2-nitro-L-phenylalanine, pentafluoro-D-phenylalanine, pentafluoro-L-phenylalanine, Phe(4-Br)—OH, D-Phe(4-Br)—OH, Phe(2-CF₃)—OH, D-Phe(2-CF₃)—OH, Phe(3-CF₃)—OH, D-Phe(3-CF₃)—OH, Phe(4-CF₃)—OH, D-Phe(4-CF₃)—OH, Phe(2-Cl)—OH, D-Phe(2-Cl)—OH, Phe(2,4-Cl₂)—OH, D-Phe(2,4-Cl₂)—OH, D-Phe(3-Cl)—OH, Phe(3,4-Cl₂)—OH, Phe(4-Cl)—OH, D-Phe(4-Cl)—OH, Phe(2-CN)—OH, D-Phe(2-CN)—OH, D-Phe(3-CN)—OH, Phe(4-CN)—OH, D-Phe(4-CN)—OH, Phe(2-Me)-OH, D-Phe(2-Me)-OH, Phe(3-Me)-OH, D-Phe(3-Me)-OH, Phe(4-Me)-OH, Phe(4-NH₂)—OH, Phe(4-NO₂)—OH, Phe(2-F)—OH, D-Phe(2-F)—OH, Phe(3-F)—OH, D-Phe(3-F)—OH, Phe(3,4-F₂)—OH, D-Phe(3,4-F₂)—OH, Phe(3,5-F₂)—OH, Phe(4-F)—OH, D-Phe(4-F)—OH, Phe(4-I)—OH, D-3,4,5-trifluorophenylalanine, p-Bromo-DL-phenylalanine, 4-Bromo-L-phenylalanine, β-phenyl-D-phenylalanine, 4-Chloro-L-phenylalanine, DL-2,3-Difluorophenylalanine, DL-3,5-Difluorophenylalanine, 3,4-Dihydroxy-L-phenylalanine, 3-(3,4-Dimethoxyphenyl)-L-alanine, N-[(9H-Fluoren-9-ylmethoxy)carbonyl]-2-methoxy-L-phenylalanine, o-Fluoro-DL-phenylalanine, m-Fluoro-L-phenylalanine, m-Fluoro-DL-phenylalanine, p-Fluoro-L-phenylalanine, p-Fluoro-DL-phenylalanine, 4-Fluoro-D-phenylalanine, 2-fluoro-L-phenylalanine methyl ester, p-fluoro-DL-Phe-OMe, D-3-bromophenylalanine, D-4-bromophenylalanine, L-β-(6-chloro-4-pyridinyl)alanine, D-3,5-difluorophenylalanine, L-3-fluorophenylalanine, L-4-fluorophenylalanine, L-β-(1H-5-indolyl)alanine, 2-nitro-L-phenylalanine, pentafluoro-L-phenylalanine, phe(3-br)-oh, Phe(4-Br)—OH, Phe(2-CF₃)—OH, D-Phe(2-CF₃)—OH, Phe(3-CF₃)—OH, D-Phe(3-CF₃)—OH, Phe(4-CF₃)—OH, D-Phe(4-CF₃)—OH, Phe(2-Cl)—OH, D-Phe(2-Cl)—OH, Phe(2,4-Cl₂)—OH, D-Phe(2,4-Cl₂)—OH, Phe(3,4-Cl₂)—OH, D-Phe(3,4-Cl₂)—OH, Phe(4-Cl)—OH, D-Phe(4-Cl)—OH, Phe(2-CN)—OH, D-Phe(2-CN)—OH, D-Phe(3-CN)—OH, Phe(4-CN)—OH, Phe(2-Me)-OH, Phe(3-Me)-OH, D-Phe(3-Me)-OH, Phe(4-NO₂)—OH, D-Phe(4-NO₂)—OH, D-Phe(2-F)—OH, Phe(3-F)—OH, D-Phe(3-F)—OH, Phe(3,4-F₂)—OH, Phe(3,5-F₂)—OH, D-Phe(4-F)—OH, Phe(4-I)—OH, D-Phe(4-I)—OH, 4-(phosphonomethyl)-Phe-OH, L-4-trifluoromethylphenylalanine, 3,4,5-trifluoro-D-phenylalanine, L-3,4,5-trifluorophenylalanine, 6-Hydroxy-DL-DOPA, 4-(Hydroxymethyl)-D-phenylalanine, N-(3-Indolylacetyl)-L-phenylalanine, p-Iodo-D-phenylalanine, 4-Iodo-L-phenylalanine, α-Methyl-D-phenylalanine, α-Methyl-L-phenylalanine, α-Methyl-DL-phenylalanine, α-Methyl-DL-phenylalanine methyl ester, 4-Nitro-D-phenylalanine, 4-Nitro-L-phenylalanine, 4-Nitro-DL-phenylalanine, (S)-(+)-4-Nitrophenylalanine methyl ester, 2-(Trifluoromethyl)-D-phenylalanine, 2-(Trifluoromethyl)-L-phenylalanine, 3-(Trifluoromethyl)-D-phenylalanine, 3-(Trifluoromethyl)-L-phenylalanine, 4-(Trifluoromethyl)-D-phenylalanine, 3,3′,5-Triiodo-L-thyronine, (R)-4-bromo-β-Phe-OH, N-Acetyl-DL-β-phenylalanine, (S)-4-bromo-β-Phe-OH, (R)-4-chloro-β-Homophe-OH, (S)-4-chloro-β-Homophe-OH, (R)-4-chloro-13-Phe-OH, (S)-4-chloro-β-Phe-OH, (S)-2-cyano-β-Homophe-OH, (R)-4-cyano-β-Homophe-OH, (S)-4-cyano-β-Homophe-OH, (R)-3-cyano-β-Phe-OH, (R)-4-cyano-β-Phe-OH, (S)-4-cyano-β-Phe-OH, (R)-3,4-dimethoxy-β-Phe-OH, (S)-3,4-dimethoxy-β-Phe-OH, (R)-4-fluoro-β-Phe-OH, (S)-4-fluoro-β-Phe-OH, (S)-4-iodo-β-Homophe-OH, (S)-3-cyano-β-Homophe-OH, (S)-3,4-difluoro-β-Homophe-OH, (R)-4-fluoro-β-Homophe-OH, (S)-β2-homophenylalanine, (R)-3-methoxy-β-Phe-OH, (S)-3-methoxy-β-Phe-OH, (R)-4-methoxy-β-Phe-OH, (S)-4-methyl-β-Homophe-OH, (R)-2-methyl-β-Phe-OH, (S)-2-methyl-β-Phe-OH, (R)-3-methyl-β-Phe-OH, (S)-3-methyl-β-Phe-OH, (R)-4-methyl-β-Phe-OH, (S)-4-methyl-β-Phe-OH, β-Phe-OH, D-β-Phe-OH, (S)-2-(trifluoromethyl)-β-Homophe-OH, (S)-2-(trifluoromethyl)-β-Homophe-OH, (S)-3-(trifluoromethyl)-β-Homophe-OH, (R)-4-(trifluoromethyl)-β-Homophe-OH, (S)-2-(trifluoromethyl)-β-Phe-OH, (R)-3-(trifluoromethyl)-β-Phe-OH, (S)-3-(trifluoromethyl)-β-Phe-OH, (R)-4-(trifluoromethyl)-β-Phe-OH, (S)-4-(trifluoromethyl)-β-Phe-OH, β-Homophe-OH, D-β-Homophe-OH, (S)-2-methyl-β-Homophe-OH, (S)-3-methyl-β-Homophe-OH, β-Phe-OH, β-D-Phe-OH, (S)-3-(trifluoromethyl)-β-Homophe-OH, L-β-Homophenylalanine, DL-β-Homophenylalanine, DL-β-Phenylalanine, DL-homophenylalanine methyl ester, D-Homophenylalanine, L-Homophenylalanine, DL-Homophenylalanine, D-Homophenylalanine ethyl ester, (R)-β²-homophenylalanine, L-α-methyl-homophenylalanine [Mhphe], L-α-methylphenylalanine [Mphe], D-α-methylphenylalanine [Dmphe], L-N-methyl-homophenylalanine [Nm phe], L-homophenylalanine [Hphe], L-N-methylphenylalanine [Nmphe], D-N-methylphenylalanine [Dnmphe], N-benzylglycine [Nphe] and D-phenylalanine [Dphe, (dF), f]. Each possibility represents a separate embodiment.

Non limiting examples for proline non-conservative amino acids are: homoproline (hPro), (4-hydroxy)Pro (4HyP), (3-hydroxy)Pro (3HyP), gamma-benzyl-proline, gamma-(2-fluoro-benzyl)-proline, gamma-(3-fluoro-benzyl)-proline, gamma-(4-fluoro-benzyl)-proline, gamma-(2-chloro-benzyl)-proline, gamma-(3-chloro-benzyl)-proline, gamma-(4-chloro-benzyl)-proline, gamma-(2-bromo-benzyl)-proline, gamma-(3-bromo-benzyl)-proline, gamma-(4-bromo-benzyl)-proline, gamma-(2-methyl-benzyl)-proline, gamma-(3-methyl-benzyl)-proline, gamma-(4-methyl-benzyl)-proline, gamma-(2-nitro-benzyl)-proline, gamma-(3-nitro-benzyl)-proline, gamma-(4-nitro-benzyl)-proline, gamma-(1-naphthalenylmethyl)-proline, gamma-(2-naphthalenylmethyl)-proline, gamma-(2,4-dichloro-benzyl)-proline, gamma-(3,4-dichloro-benzyl)-proline, gamma-(3,4-difluoro-benzyl)-proline, gamma-(2-trifluoro-methyl-benzyl)-proline, gamma-(3-trifluoro-methyl-benzyl)-proline, gamma-(4-trifluoro-methyl-benzyl)-proline, gamma-(2-cyano-benzyl)-proline, gamma-(3-cyano-benzyl)-proline, gamma-(4-cyano-benzyl)-proline, gamma-(2-iodo-benzyl)-proline, gamma-(3-iodo-benzyl)-proline, gamma-(4-iodo-benzyl)-proline, gamma-(3-phenyl-allyl-benzyl)-proline, gamma-(3-phenyl-propyl-benzyl)-proline, gamma-(4-tert-butyl-benzyl)-proline, gamma-benzhydryl-proline, gamma-(4-biphenyl-methyl)-proline, gamma-(4-thiazolyl-methyl)-proline, gamma-(3-benzothienyl-methyl)-proline, gamma-(2-thienyl-methyl)-proline, gamma-(3-thienyl-methyl)-proline, gamma-(2-furanyl-methyl)-proline, gamma-(2-pyridinyl-methyl)-proline, gamma-(3-pyridinyl-methyl)-proline, gamma-(4-pyridinyl-methyl)-proline, gamma-allyl-proline, gamma-propynyl-proline, alpha-modified-proline residues, pipecolic acid, azetidine-3-carboxylicacid, L-β-Homoproline, L-β³-homoproline, L-β-Homohydroxyproline, hydroxyproline [Hyp], L-□-methylproline [Mpro], D-□-methylproline [Dmpro], L-N-methylproline [Nmpro], D-N-methylproline [Dnmpro], and D-proline [Dpro, (dP), p]. Each possibility represents a separate embodiment.

Non limiting examples for serine non-conservative amino acids are: (2R,3S)-3-phenylisoserine, D-cycloserine, L-Isoserine, DL-Isoserine, DL-3-Phenylserine, L-β-Homoserine, D-Homoserine, D-Homoserine, L-3-Homoserine, L-homoserine, L-α-methylserine [Mser], D-α-methylserine [Dmser], L-N-methylserine [Nmser], D-N-methylserine [Dnmser], D-serine [Dser, (dS), s], N-(hydroxymethyl)glycine [Nser] and phosphoserine [pSer]. Each possibility represents a separate embodiment.

Non limiting examples for threonine non-conservative amino acids are: L-allo-Threonine, D-Thyroxine, L-β-Homothreonine, L-α-methylthreonine [Mthr], D-α-methylthreonine [Dmthr], L-N-methylthreonine [Nmthr], D-N-methylthreonine [Dnmthr], D-threonine [Dthr, (dT), t], N-(1-hydroxyethyl)glycine [Nthr] and phosphothreonine [pThr]. Each possibility represents a separate embodiment.

Non limiting examples for tryptophan non-conservative amino acids are: 5-Fluoro-L-tryptophan, 5-Fluoro-DL-tryptophan, 5-Hydroxy-L-tryptophan, 5-Methoxy-DL-tryptophan, L-abrine, 5-Methyl-DL-tryptophan, H-Tpi-OMe. β-Homotrp-OMe, L-β-Homotryptophan, L-α-methyltryptophan [Mtrp], D-α-methyltryptophan [Dmtrp], L-N-methyltryptophan [Nmtrp], D-N-methyltryptophan [Dnmtrp], N-(3-indolylethyl)glycine [Nhtrp], D-tryptophan [Dap, (dW), w]. Each possibility represents a separate embodiment.

Non limiting examples for tyrosine non-conservative amino acids are: 3,5 diiodotyrosine (3,5-dITyr), 3,5 diBromotyrosine (3,5-dBTyr), homotyrosine, D-tyrosine, 3-amino-L-tyrosine, 3-amino-D-tyrosine, 3-iodo-L-tyrosine, 3-iodo-D-tyrosine, 3-methoxy-L-tyrosine, 3-methoxy-D-tyrosine, L-thyroxine, D-thyroxine, L-thyronine, D-thyronine, O-methyl-L-tyrosine, O-methyl-D-tyrosine, D-thyronine, O-ethyl-L-tyrosine, O-ethyl-D-tyrosine, 3,5,3′-triiodo-L-thyronine, 3,5,3′-triiodo-D-thyronine, 3,5-diiodo-L-thyronine, 3,5-diiodo-D-thyronine, D-meta-tyrosine, L-meta-tyrosine, D-ortho-tyrosine, L-ortho-tyrosine, phenylalanine, substituted phaenylalanine, N-nitro phenylalanine, p-nitro phenylalanine, 3-chloro-Dtyr-oh, Tyr(3,5-diI), 3-Chloro-L-tyrosine, Tyr(3-NO₂)—OH, Tyr(3,5-diI)—OH, N-Me-Tyr-OH, α-Methyl-DL-tyrosine, 3-Nitro-L-tyrosine, DL-o-Tyrosine, β-Homotyr-OH, (R)-β-Tyr-OH, (S)-β-Tyr-OH, L-α-methyltyrosine [Mtyr], D-α-methyltyrosine [Dmtyr], L-N-methyltyrosine [Nmtyr], D-N-methyltyrosine [Dnmtyr], D-tyrosine [Dtyr, (dY), y], O-methyl-tyrosine, and phosphotyrosine [pTyr]. Each possibility represents a separate embodiment.

Non limiting examples for valine non-conservative amino acids are: 3-Fluoro-DL-valine, 4,4,4,4′,4′,4′-Hexafluoro-DL-valine, D-valine [Dval, (dV), v], N-Me-Val-OH [Nmval], N-Me-Val-OH, L-α-methylvaline [Mval], D-α-methylvaline [Dmval], (R)-(+)-α-Methylvaline, (S)-(−)-α-Methylvaline and D-N-methylvaline [Dnmval]. Each possibility represents a separate embodiment.

Other non-natural amino acids that may be substituted as non-conservative replacements include: Ornithine and its modifications: D-Ornithine [Dorn], L-Ornithine [Orn], DL-Ornithine, L-α-methylornithine [Morn], D-α-methylornithine [Dmorn], L-N-methylornithine [Nmorn], D-N-methylornithine [Dnmorn] and N-(3-aminopropyl)glycine [Norn]. Each possibility represents a separate embodiment.

Alicyclic amino acids: L-2,4-Diaminobutyric acid, L-2,3-Diaminopropionic Acid, N-Me-Aib-OH, (R)-2-(amino)-5-hexynoic acid, piperidine-2-carboxylic acid, aminonorbornyl-carboxylate [Norb], alpha-aminobutyric acid [Abu], aminocyclopropane-carboxylate [Cpro], (cis)-3-Aminobicyclo[2.2.1]heptane-2-carboxylic acid, exo-cis-3-Aminobicyclo[2.2.1]hept-5-ene-2-carboxylic acid, 1-Amino-1-cyclobutanecarboxylic acid, cis-2-Aminocycloheptanecarboxylic acid, 1-Aminocyclohexanecarboxylic acid, cis-2-Aminocyclohexanecarboxylic acid, trans-2-Aminocyclohexanecarboxylic acid, cis-6-Amino-3-cyclohexene-1-carboxylic acid, 2-(1-Aminocyclohexyl)acetic acid, cis-2-Amino-1-cyclooctanecarboxylic acid, cis-2-Amino-3-cyclooctene-1-carboxylic acid, (1R,2S)-(−)-2-Amino-1-cyclopentanecarboxylic acid, (1S,2R)-(+)-2-Amino-1-cyclopentanecarboxylic acid, cis-2-Amino-1-cyclopentanecarboxylic acid, 2-(1-Aminocyclopentyl)acetic acid, cis-2-Amino-2-methylcyclohexanecarboxylic acid, cis-2-Amino-2-methylcyclopentanecarboxylic acid, 3-Amino-3-(4-nitrophenyl)propionic acid, 3-Azetidinecarboxylic acid, amchc-oh, 1-aminocyclobutane carboxylic acid, 1-(amino)cyclohexanecarboxylic acid, cis-2-(amino)-cyclohexanecarboxylic acid, trans-2-(amino)-cyclohexanecarboxylic acid, cis-4-(amino)cyclohexanecarboxylic acid, trans-4-(amino)cyclohexanecarboxylic acid, (±)-cis-2-(amino)-3-cyclohexene-1-carboxylic acid, (±)-cis-6-(amino)-3-cyclohexene-1-carboxylic acid, 2-(1-aminocyclohexyl)acetic acid, cis-[4-(amino)cyclohexyl]acetic acid, 1-(amino)cyclopentanecarboxylic acid, (±)-cis-2-(amino)cyclopentanecarboxylic acid, (1R,4S)-(+)-4-(amino)-2-cyclopentene-1-carboxylic acid, (±)-cis-2-(amino)-3-cyclopentene-1-carboxylic acid, 2-(1-aminocyclopentyl)acetic acid, 1-(amino)cyclopropanecarboxylic acid, Ethyl 1-aminocyclopropanecarboxylate, 1,2-trans-achec-oh, 1-(amino)cyclobutanecarboxylic acid, 1-(amino)cyclohexanecarboxylic acid, cis-2-(amino)-cyclohexanecarboxylic acid, trans-2-(amino)cyclohexanecarboxylic acid, cis-4-(amino)cyclohexanecarboxylic acid, trans-4-(amino)cyclohexanecarboxylic acid, cis-[4-(amino)cyclohexyl]acetic acid, 1-(amino)cyclopentanecarboxylic acid, (1R,4S)-(+)-4-(amino)-2-cyclopentene-1-carboxylic acid, (1S,4R)-(−)-4-(amino)-2-cyclopentene-1-carboxylic acid, 1-(amino)cyclopropanecarboxylic acid, trans-4-(aminomethyl)cyclohexanecarboxylic acid, β-Dab-OH, 3-Amino-3-(3-bromophenyl)propionic acid, 3-Aminobutanoic acid, cis-2-Amino-3-cyclopentene-1-carboxylic acid, DL-3-Aminoisobutyric acid, (R)-3-Amino-2-phenylpropionic acid, (±)-3-(amino)-4-(4-biphenylyl)butyric acid, cis-3-(amino)cyclohexanecarboxylic acid, (1S,3R)-(+)-3-(amino)cyclopentanecarboxylic acid, (2R,3R)-3-(amino)-2-hydroxy-4-phenylbutyric acid, (2S,3R)-3-(amino)-2-hydroxy-4-phenylbutyric acid, 2-(aminomethyl)phenylacetic acid, (R)-3-(amino)-2-methylpropionic acid, (S)-3-(amino)-2-methylpropionic acid, (R)-3-(amino)-4-(2-naphthyl)butyric acid, (S)-3-(amino)-4-(2-naphthyl)butyric acid, (R)-3-(amino)-5-phenylpentanoic acid, (R)-3-(amino)-2-phenylpropionic acid, Ethyl 3-(benzylamino)propionate, cis-3-(amino)cyclohexanecarboxylic acid, (S)-3-(amino)-5-hexenoic acid, (R)-3-(amino)-2-methylpropionic acid, (S)-3-(amino)-2-methylpropionic acid, (R)-3-(amino)-4-(2-naphthyl)butyric acid, (S)-3-(amino)-4-(2-naphthyl)butyric acid, (R)-(−)-Pyrrolidine-3-carboxylic acid, (S)-(+)-Pyrrolidine-3-carboxylic acid, N-methyl-γ-aminobutyrate [Nmgabu], γ-aminobutyric acid [Gabu], N-methyl-α-amino-α-methylbutyrate [Nmaabu], α-amino-α-methylbutyrate [Aabu], N-methyl-α-aminoisobutyrate [Nmaib], α-aminoisobutyric acid [Aib], α-methyl-γ-aminobutyrate [Mgabu]. Each possibility represents a separate embodiment.

Phenyl glycine and its modifications: Phg-OH, D-Phg-OH, 2-(piperazino)-2-(3,4-dimethoxyphenyl)acetic acid, 2-(piperazino)-2-(2-fluorophenyl)acetic acid, 2-(4-piperazino)-2-(3-fluorophenyl)acetic acid, 2-(4-piperazino)-2-(4-methoxyphenyl)acetic acid, 2-(4-piperazino)-2-(3-pyridyl)acetic acid, 2-(4-piperazino)-2-[4-(trifluoromethyl)phenyl]acetic acid, L-(+)-2-Chlorophenylglycine, (±)-2-Chlorophenylglycine, (±)-4-Chlorophenylglycine, (R)-(−)-2-(2,5-Dihydrophenyl)glycine, (R)-(−)-N-(3,5-Dinitrobenzoyl)-α-phenylglycine, (S)-(+)-N-(3,5-Dinitrobenzoyl)-α-phenylglycine, 2,2-Diphenylglycine, 2-Fluoro-DL-α-phenylglycine, 4-Fluoro-D-α-phenylglycine, 4-Hydroxy-D-phenylglycine, 4-Hydroxy-L-phenylglycine, 2-Phenylglycine, D-(−)-α-Phenylglycine, D-(−)-α-Phenylglycine, DL-α-Phenylglycine, L-(+)-α-Phenylglycine, N-Phenylglycine, (R)-(−)-2-Phenylglycine methyl ester, (S)-(+)-2-Phenylglycine methyl ester, 2-Phenylglycinonitrile hydrochloride, α-Phenylglycinonitrile, 3-(Trifluoromethyl)-DL-phenylglycine, and 4-(Trifluoromethyl)-L-phenylglycine. Each possibility represents a separate embodiment.

Penicillamine and its modifications: N-Acetyl-D-penicillamine, D-Penicillamine, L-Penicillamine [Pen], DL-Penicillamine α-methylpenicillamine [Mpen], N-methylpenicillamine [Nmpen]. Each possibility represents a separate embodiment.

β-Homopyrrolidine. Each possibility represents a separate embodiment.

Aromatic amino acids: 3-Acetamidobenzoic acid, 4-Acetamidobenzoic acid, 4-Acetamido-2-methylbenzoic acid, N-Acetylanthranilic acid, 3-Aminobenzoic acid, 3-Aminobenzoic acid hydrochloride, 4-Aminobenzoic acid, 4-Aminobenzoic acid, 4-Aminobenzoic acid, 4-Aminobenzoic acid, 4-Aminobenzoic acid, 4-Aminobenzoic acid, 2-Aminobenzophenone-2′-carboxylic acid, 2-Amino-4-bromobenzoic acid, 2-Amino-5-bromobenzoic acid, 3-Amino-2-bromobenzoic acid, 3-Amino-4-bromobenzoic acid, 3-Amino-5-bromobenzoic acid, 4-Amino-3-bromobenzoic acid, 5-Amino-2-bromobenzoic acid, 2-Amino-3-bromo-5-methylbenzoic acid, 2-Amino-3-chlorobenzoic acid, 2-Amino-4-chlorobenzoic acid, 2-Amino-5-chlorobenzoic acid, 2-Amino-5-chlorobenzoic acid, 2-Amino-6-chlorobenzoic acid, 3-Amino-2-chlorobenzoic acid, 3-Amino-4-chlorobenzoic acid, 4-Amino-2-chlorobenzoic acid, 4-Amino-3-chlorobenzoic acid, 5-Amino-2-chlorobenzoic acid, 5-Amino-2-chlorobenzoic acid, 4-Amino-5-chloro-2-methoxybenzoic acid, 2-Amino-5-chloro-3-methylbenzoic acid, 3-Amino-2,5-dichlorobenzoic acid, 4-Amino-3,5-dichlorobenzoic acid, 2-Amino-4,5-dimethoxybenzoic acid, 4-(2-Aminoethyl)benzoic acid hydrochloride, 2-Amino-4-fluorobenzoic acid, 2-Amino-5-fluorobenzoic acid, 2-Amino-6-fluorobenzoic acid, 4-Amino-2-fluorobenzoic acid, 2-Amino-5-hydroxybenzoic acid, 3-Amino-4-hydroxybenzoic acid, 4-Amino-3-hydroxybenzoic acid, 2-Amino-5-iodobenzoic acid, 5-Aminoisophthalic acid, 2-Amino-3-methoxybenzoic acid, 2-Amino-4-methoxybenzoic acid, 2-Amino-5-methoxybenzoic acid, 3-Amino-2-methoxybenzoic acid, 3-Amino-4-methoxybenzoic acid, 3-Amino-5-methoxybenzoic acid, 4-Amino-2-methoxybenzoic acid, 4-Amino-3-methoxybenzoic acid, 5-Amino-2-methoxybenzoic acid, 2-Amino-3-methylbenzoic acid, 2-Amino-5-methylbenzoic acid, 2-Amino-6-methylbenzoic acid, 3-(Aminomethyl)benzoic acid, 3-Amino-2-methylbenzoic acid, 3-Amino-4-methylbenzoic acid, 4-(Aminomethyl)benzoic acid, 4-Amino-2-methylbenzoic acid, 4-Amino-3-methylbenzoic acid, 5-Amino-2-methylbenzoic acid, 3-Amino-2-naphthoic acid, 6-Amino-2-naphthoic acid, 2-Amino-3-nitrobenzoic acid, 2-Amino-5-nitrobenzoic acid, 2-Amino-5-nitrobenzoic acid, 4-Amino-3-nitrobenzoic acid, 5-Amino-2-nitrobenzoic acid, 3-(4-Aminophenyl)propionic acid, 3-Aminophthalic acid, 4-Aminophthalic acid, 3-Aminosalicylic acid, 4-Aminosalicylic acid, 5-Aminosalicylic acid, 5-Aminosalicylic acid, 2-Aminoterephthalic acid, 2-Amino-3,4,5,6-tetrafluorobenzoic acid, 4-Amino-2,3,5,6-tetrafluorobenzoic acid, (R)-2-Amino-1,2,3,4-tetrahydronaphthalene-2-carboxylic acid, (S)-2-Amino-1,2,3,4-tetrahydro-2-naphthalenecarboxylic acid, 2-Amino-3-(trifluoromethyl)benzoic acid, 2-Amino-3-(trifluoromethyl)benzoic acid, 3-Amino-5-(trifluoromethyl)benzoic acid, 5-Amino-2,4,6-triiodoisophthalic acid, 2-Amino-3,4,5-trimethoxybenzoic acid, 2-Anilinophenylacetic acid, 2-Abz-OH, 3-Abz-OH, 4-Abz-OH, 2-(aminomethyl)benzoic acid, 3-(aminomethyl)benzoic acid, 4-(aminomethyl)benzoic acid, tert-Butyl 2-aminobenzoate, tert-Butyl 3-aminobenzoate, tert-Butyl 4-aminobenzoate, 4-(Butylamino)benzoic acid, 2,3-Diaminobenzoic acid, 3,4-Diaminobenzoic acid, 3,5-Diaminobenzoic acid, 3,5-Diaminobenzoic acid, 3,5-Dichloroanthranilic acid, 4-(Diethylamino)benzoic acid, 4,5-Difluoroanthranilic acid, 4-(Dimethylamino)benzoic acid, 4-(Dimethylamino)benzoic acid, 3,5-Dimethylanthranilic acid, 5-Fluoro-2-methoxybenzoic acid, 2-Abz-OH, 3-Abz-OH, 4-Abz-OH, 3-(aminomethyl)benzoic acid, 4-(aminomethyl)benzoic acid, 4-(2-hydrazino)benzoic acid, 3-Hydroxyanthranilic acid, 3-Hydroxyanthranilic acid, Methyl 3-aminobenzoate, 3-(Methylamino)benzoic acid, 4-(Methylamino)benzoic acid, Methyl 2-amino-4-chlorobenzoate, Methyl 2-amino-4,5-dimethoxybenzoate, 4-Nitroanthranilic acid, N-Phenylanthranilic acid, N-Phenylanthranilic acid, and Sodium 4-aminosalicylate. Each possibility represents a separate embodiment.

Other amino acids: (S)-α-Amino-γ-butyrolactone, DL-2-Aminocaprylic acid, 7-Aminocephalosporanic acid, 4-Aminocinnamic acid, (S)-(+)-α-Aminocyclohexanepropionic acid, (R)-Amino-(4-hydroxyphenyl)acetic acid methyl ester, 5-Aminolevulinic acid, 4-Amino-nicotinic acid, 3-Aminophenylacetic acid, 4-Aminophenylacetic acid, 2-Amino-2-phenylbutyric acid, 4-(4-Aminophenyl)butyric acid, 2-(4-Aminophenylthio)acetic acid, DL-α-Amino-2-thiopheneacetic acid, 5-Aminovaleric acid, 8-Benzyl (S)-2-aminooctanedioate, 4-(amino)-1-methylpyrrole-2-carboxylic acid, 4-(amino)tetrahydrothiopyran-4-carboxylic acid, (1R,3S,4S)-2-azabicyclo[2.2.1]heptane-3-carboxylic acid, L-azetidine-2-carboxylic acid, azetidine-3-carboxylic acid, 4-(amino)piperidine-4-carboxylic acid, diaminoacetic acid, Inp-OH, (R)-Nip-OH, (S)-4-oxopiperidine-2-carboxylic acid, 2-(4-piperazino)-2-(4-fluorophenyl)acetic acid, 2-(4-piperazino)-2-phenylacetic acid, 4-piperidineacetaldehyde, 4-piperidylacetic acid, (−)-L-thioproline, Tle-OH, 3-piperidinecarboxylic acid, L-(+)-Canavanine, (±)-Carnitine, Chlorambucil, 2,6-Diaminopimelic acid, meso-2,3-Diaminosuccinic acid, 4-(Dimethylamino)cinnamic acid, 4-(Dimethylamino)phenylacetic acid, Ethyl (S)—N-Boc-piperidine-3-carboxylate, Ethyl piperazinoacetate, 4-[2-(amino)ethyl]piperazin-1-ylacetic acid, (R)-4-(amino)-5-phenylpentanoic acid, (S)-azetidine-2-carboxylic acid, azetidine-3-carboxylic acid, guvacine, Inp-OH, (R)-Nip-OH, DL-Nip-OH, 4-phenyl-piperidine-4-carboxylic acid, 1-piperazineacetic acid, 4-piperidineacetic acid, (R)-piperidine-2-carboxylic acid, (S)-piperidine-2-carboxylic acid, (S)-1,2,3,4-tetrahydronorharmane-3-carboxylic acid, Tic-OH, D-Tic-OH, Iminodiacetic acid, Indoline-2-carboxylic acid, DL-Kynurenine, L-aziridine-2-carboxylate, Methyl 4-aminobutyrate, (S)-2-Piperazinecarboxylic acid, 2-(1-Piperazinyl)acetic acid, (R)-(−)-3-Piperidinecarboxylic acid, 2-Pyrrolidone-5-carboxylic acid, (R)-(+)-2-Pyrrolidone-5-carboxylic acid, (R)-1,2,3,4-Tetrahydro-3-isoquinolinecarboxylic acid, (S)-1,2,3,4-Tetrahydro-3-isoquinolinecarboxylic acid, L-4-Thiazolidinecarboxylic acid, (4R)-(−)-2-Thioxo-4-thiazolidinecarboxylic acid, hydrazinoacetic acid, and 3,3′,5-Triiodo-L-thyronine. Each possibility represents a separate embodiment.

The present disclosure provides peptides comprising peptidomimetic compounds having further improved stability and cell permeability properties. Some embodiments comprise a peptide according to any of SEQ ID NO: 1-210, wherein one of more peptide bonds (—CO—NH—) within the peptide may be substituted, for example, by N-methylated amide bonds (—N(CH3)-CO—), ester bonds (—C(═O)—O—), ketomethylene bonds (—CO—CH₂—), sulfinylmethylene bonds (—S(═O)—CH₂—), α-aza bonds (—NH—N(R)—CO—), wherein R is any alkyl (e.g., methyl), amine bonds (—CH₂—NH—), sulfide bonds (—CH₂—S—), ethylene bonds (—CH₂CH₂—), hydroxyethylene bonds (—CH(OH)—CH₂—), thioamide bonds (—CS—NH—), olefinic double bonds (—CH═CH—), fluorinated olefinic double bonds (—CF=CH—), or retro amide bonds (—NH—CO—), peptide derivatives (—N(R^{x})—CH₂—CO—), wherein R^{x} is the “normal” side chain, naturally present on the carbon atom. These modifications can occur at any of the bonds along the peptide chain and even at several (2-3) bonds at the same time.

In exemplary embodiments, the peptide comprises at least seven or eight amino acids connected via peptide bonds. In exemplary aspects, the peptide is at least about 9 amino acids in length, at least about 10 amino acids in length, at least about 11 amino acids in length, at least about 12 amino acids in length, or at least about 13 amino acids in length. In exemplary aspects, the peptide is at least about 14 amino acids in length, at least about 15 amino acids in length, at least about 16 amino acids in length, or at least about 17 amino acids in length. In exemplary aspects, the peptide is at least about 18 amino acids in length, at least about 19 amino acids in length, or at least about 20 amino acids in length. In exemplary aspects, the peptide is less than about 50 amino acids in length, less than about 40 amino acids, or less than about 30 amino acids, or less than about 25 amino acids in length. In exemplary aspects, the peptide is about 8 to about 30 amino acids in length or about 10 to about 30 amino acids in length. In exemplary aspects, the peptide is about 10 to about 25 amino acids in length, about 14 to about 25 amino acids in length, about 18 to about 30 amino acids in length, or about 18 to about 26 amino acids in length. In exemplary aspects, the peptide is 22-24, 23-24, or 24-25 amino acids in length. In some embodiments, the peptide is a 22-mer, 23-mer, 24-mer, 25-mer, 26-mer, 27-mer, 28-mer, 29-mer or 30-mer.

The peptides of some embodiments are preferably utilized in a linear form, although it will be appreciated that in cases where cyclization does not severely interfere with peptide characteristics, cyclic forms of the peptide can also be utilized and are contemplated as embodiments.

According to some embodiments conjugates comprising any of the peptides and analogs described herein conjugated to a moiety for extending half-life or increasing cell penetration. For example, the half-life extending moiety may be a peptide or protein and the conjugate is a fusion protein or chimeric polypeptide. Alternatively, the half-life extending moiety may be a polymer, e.g., a polyethylene glycol. The present disclosures furthermore provide dimers and multimers comprising any of the peptides and analogs described herein.

Any moiety known in the art to facilitate actively or passively or enhance permeability of the peptides into cells may be used for conjugation with the peptide core. Non-limitative examples include: hydrophobic moieties such as fatty acids, steroids and bulky aromatic or aliphatic compounds; moieties which may have cell-membrane receptors or carriers, such as steroids, vitamins and sugars, natural and non-natural amino acids and transporter peptides. According to a preferred embodiment, the hydrophobic moiety is a lipid moiety or an amino acid moiety. The permeability-enhancing moiety may be connected to any position in the peptide moiety, directly or through a spacer or linker, preferably to the amino terminus of the peptide moiety. The hydrophobic moiety may preferably comprise a lipid moiety or an amino acid moiety. According to a specific embodiment the hydrophobic moiety is selected from the group consisting of: phospholipids, steroids, sphingosines, ceramides, octyl-glycine, 2-cyclohexylalanine, benzolylphenylalanine, propionoyl (C₃); butanoyl (C₄); pentanoyl (C₅); caproyl (C₆); heptanoyl (C₇); capryloyl (C₈); nonanoyl (C₉); capryl (C₁₀); undecanoyl (C₁₁); lauroyl (C₁₂); tridecanoyl (C₁₃); myristoyl (C₁₄); pentadecanoyl (C₁₅); palmitoyl (C₁₆); phtanoyl ((CH₃)₄); heptadecanoyl (C₁₆); stearoyl (C₁₈); nonadecanoyl (C₁₉); arachidoyl (C₂₀); heniecosanoyl (C₂₁); behenoyl (C₂₂); trucisanoyl (C₂₃); and lignoceroyl (C₂₄); wherein said hydrophobic moiety is attached to said chimeric polypeptide with amide bonds, sulfhydryls, amines, alcohols, phenolic groups, or carbon-carbon bonds. Other examples of lipidic moieties which may be used include: Lipofectamine, Transfectace, Transfectam, Cytofectin, DMRIE, DLRIE, GAP-DLRIE, DOTAP, DOPE, DMEAP, DODMP, DOPC, DDAB, DOSPA, EDLPC, EDMPC, DPH, TMADPH, CTAB, lysyl-PE, DC-Cho, -alanyl cholesterol; DCGS, DPPES, DCPE, DMAP, DMPE, DOGS, DOHME, DPEPC, Pluronic, Tween, BRIJ, plasmalogen, phosphatidylethanolamine, phosphatidylcholine, glycerol-3-ethylphosphatidylcholine, dimethyl ammonium propane, trimethyl ammonium propane, diethylammonium propane, triethylammonium propane, dimethyldioctadecylammonium bromide, a sphingolipid, sphingomyelin, a lysolipid, a glycolipid, a sulfatide, a glycosphingolipid, cholesterol, cholesterol ester, cholesterol salt, oil, N-succinyldioleoylphosphatidylethanolamine, 1,2-dioleoyl-glycerol, 1,3-dipalmitoyl-2-succinylglycerol, 1,2-dipalmitoyl-3-succinylglycerol, 1-hexadecyl-2-palmitoylglycerophosphatidylethanolamine, palmitoylhomocystiene, N,N′-Bis (dodecyaminocarbonylmethylene)-N,N′-bis((-N,N,N-trimethylammoniumethyl-ami nocarbonylmethylene)ethylenediamine tetraiodide; N,N″-Bis(hexadecylaminocarbonylmethylene)-N,N′, N″-tris((-N,N,N-trimethylammonium-ethylaminocarbonylmethylenediethylenetri amine hexaiodide; N,N′-Bis(dodecylaminocarbonylmethylene)-N,N″-bis((-N,N,N-trimethylammonium ethylaminocarbonylmethylene)cyclohexylene-1,4-diamine tetraiodide; 1,7,7-tetra-((-N,N,N,N-tetramethylammoniumethylamino-carbonylmethylene)-3-hexadecylarninocarbonyl-methylene-1,3,7-triaazaheptane heptaiodide; N,N,N′,N′-tetra((-N,N,N-trimethylammonium-ethylaminocarbonylmethylene)-N′-(1,2-dioleoylglycero-3-phosphoethanolamino carbonylmethylene)diethylenetriamine tetraiodide; dioleoylphosphatidylethanolamine, a fatty acid, a lysolipid, phosphatidylcholine, phosphatidylethanolamine, phosphatidylserine, phosphatidylglycerol, phosphatidylinositol, a sphingolipid, a glycolipid, a glucolipid, a sulfatide, a glycosphingolipid, phosphatidic acid, palmitic acid, stearic acid, arachidonic acid, oleic acid, a lipid bearing a polymer, a lipid bearing a sulfonated saccharide, cholesterol, tocopherol hemisuccinate, a lipid with an ether-linked fatty acid, a lipid with an ester-linked fatty acid, a polymerized lipid, diacetyl phosphate, stearylamine, cardiolipin, a phospholipid with a fatty acid of 6-8 carbons in length, a phospholipid with asymmetric acyl chains, 6-(5-cholesten-3b-yloxy)-1-thio-b-D-galactopyranoside, digalactosyldiglyceride, 6-(5-cholesten-3b-yloxy)hexyl-6-amino-6-deoxy-1-thio-b-D-galactopyranoside, 6-(5-cholesten-3b-yloxy)hexyl-6-amino-6-deoxyl-1-thio-α-D-mannopyranoside, 12-(((7′-diethylamino-coumarin-3-yl)carbonyl)methylamino)-octadecanoic acid; N-[12-(((7′-diethylaminocoumarin-3-yl)carbonyl)methyl-amino) octadecanoyl]-2-aminopalmitic acid; cholesteryl-4′-trimethyl-amino)butanoate; N-succinyldioleoyl-phosphatidylethanolamine; 1,2-dioleoyl-glycerol; 1,2-dipalmitoyl-3-succinyl-glycerol; 1,3-dipalmitoyl-2-succinylglycerol, 1-hexadecyl-2-palmitoylglycero-phosphoethanolamine, and palmitoylhomocysteine.

The peptides disclosed herein may be conjugated to one or more moieties that cause the conjugate to function as a prodrug. For example, the N-amino acid related moieties described in U.S. Pat. No. 8,969,288 and US Pub. 20160058881 can be conjugated to the peptides disclosed herein and such conjugates are included in this disclosure.

According to some embodiments the peptides may be attached (either covalently or non-covalently) to a penetrating agent. As used herein the phrase “penetrating agent” refers to an agent which enhances translocation of any of the attached peptide across a cell membrane. Typically, peptide based penetrating agents have an amino acid composition containing either a high relative abundance of positively charged amino acids such as lysine or arginine, or have sequences that contain an alternating pattern of polar/charged amino acids and non-polar, hydrophobic amino acids. By way of a non-limiting example, cell penetrating peptide (CPP) sequences may be used in order to enhance intracellular penetration. CPPs may include short and long versions of the protein transduction domain (PTD) of HIV TAT protein, such as for example, YARAAARQARA (SEQ ID NO: 211), YGRKKRR (SEQ ID NO: 212), YGRKKRRQRRR (SEQ ID NO: 213), or RRQRR (SEQ ID NO: 214]. However, the disclosure is not so limited, and any suitable penetrating agent may be used, as known by those of skill in the art. Another method of enhancing cell penetration is via N-terminal myristoilation. In this protein modification, a myristoyl group (derived from myristic acid) is covalently attached via an amide bond to the alpha-amino group of an N-terminal amino acid of the peptide.

According to some embodiments the peptide is modified to include a duration enhancing moiety. The duration enhancing moiety can be a water soluble polymer, or a long chain aliphatic group. In some embodiments, a plurality of duration enhancing moieties may be attached to the peptide, in which case each linker to each duration enhancing moiety is independently selected from the linkers described herein.

According to some embodiments the amino terminus of the peptide is modified, e.g. acylated. According to additional embodiments the carboxy terminus is modified, e.g., it may be acylated, amidated, reduced or esterified. In accordance with some embodiments, the peptide comprises an acylated amino acid (e.g., a non-coded acylated amino acid (e.g., an amino acid comprising an acyl group which is non-native to a naturally-occurring amino acid)). In accordance with one embodiment, the peptide comprises an acyl group which is attached to the peptide via an ester, thioester, or amide linkage for purposes of prolonging half-life in circulation and/or delaying the onset of and/or extending the duration of action and/or improving resistance to proteases. Acylation can be carried out at any position within the peptide, (e.g., the amino acid at the C-terminus), provided that activity is retained, if not enhanced. The peptide in some embodiments can be acylated at the same amino acid position where a hydrophilic moiety is linked, or at a different amino acid position. The acyl group can be covalently linked directly to an amino acid of the peptide, or indirectly to an amino acid of the peptide via a spacer, wherein the spacer is positioned between the amino acid of the peptide and the acyl group.

In specific aspects, the peptide is modified to comprise an acyl group by direct acylation of an amine, hydroxyl, or thiol of a side chain of an amino acid of the peptide. In this regard, the acylated peptide can comprise the amino acid sequence of any of SEQ ID NO: 1-210, or a modified amino acid sequence thereof comprising one or more of the amino acid modifications described herein.

In some embodiments, the peptide comprises a spacer between the analog and the acyl group. In some embodiments, the peptide is covalently bound to the spacer, which is covalently bound to the acyl group. In some embodiments, the spacer is an amino acid comprising a side chain amine, hydroxyl, or thiol, or a dipeptide or tripeptide comprising an amino acid comprising a side chain amine, hydroxyl, or thiol. The amino acid to which the spacer is attached can be any amino acid (e.g., a singly or doubly α-substituted amino acid) comprising a moiety which permits linkage to the spacer. For example, an amino acid comprising a side chain NH₂, —OH, or —COOH (e.g., Lys, Orn, Ser, Asp, or Glu) is suitable. In some embodiments, the spacer is an amino acid comprising a side chain amine, hydroxyl, or thiol, or a dipeptide or tripeptide comprising an amino acid comprising a side chain amine, hydroxyl, or thiol. When acylation occurs through an amine group of a spacer, the acylation can occur through the alpha amine of the amino acid or a side chain amine. In the instance in which the alpha amine is acylated, the amino acid of the spacer can be any amino acid. For example, the amino acid of the spacer can be a hydrophobic amino acid, e.g., Gly, Ala, Val, Leu, Ile, Trp, Met, Phe, Tyr, 6-amino hexanoic acid, 5-aminovaleric acid, 7-aminoheptanoic acid, and 8-aminooctanoic acid. Alternatively, the amino acid of the spacer can be an acidic residue, e.g., Asp, Glu, homoglutamic acid, homocysteic acid, cysteic acid, gamma-glutamic acid. In the instance in which the side chain amine of the amino acid of the spacer is acylated, the amino acid of the spacer is an amino acid comprising a side chain amine. In this instance, it is possible for both the alpha amine and the side chain amine of the amino acid of the spacer to be acylated, such that the peptide is diacylated. Embodiments include such diacylated molecules. When acylation occurs through a hydroxyl group of a spacer, the amino acid or one of the amino acids of the dipeptide or tripeptide can be Ser. When acylation occurs through a thiol group of a spacer, the amino acid or one of the amino acids of the dipeptide or tripeptide can be Cys. In some embodiments, the spacer is a hydrophilic bifunctional spacer. In certain embodiments, the hydrophilic bifunctional spacer comprises two or more reactive groups, e.g., an amine, a hydroxyl, a thiol, and a carboxyl group or any combinations thereof. In certain embodiments, the hydrophilic bifunctional spacer comprises a hydroxyl group and a carboxylate. In other embodiments, the hydrophilic bifunctional spacer comprises an amine group and a carboxylate. In other embodiments, the hydrophilic bifunctional spacer comprises a thiol group and a carboxylate.

In a specific embodiment, the spacer comprises an amino poly(alkyloxy)carboxylate. In this regard, the spacer can comprise, for example, NH₂(CH₂CH₂O)ₙ(CH₂)ₘCOOH, wherein m is any integer from 1 to 6 and n is any integer from 2 to 12, such as, e.g., 8-amino-3,6-dioxaoctanoic acid, which is commercially available from Peptides International, Inc. (Louisville, Ky.). In some embodiments, the spacer is a hydrophobic bifunctional spacer. Hydrophobic bifunctional spacers are known in the art. See, e.g., Bioconjugate Techniques, G. T. Hermanson (Academic Press, San Diego, Calif., 1996), which is incorporated by reference in its entirety. In certain embodiments, the hydrophobic bifunctional spacer comprises two or more reactive groups, e.g., an amine, a hydroxyl, a thiol, and a carboxyl group or any combinations thereof. In certain embodiments, the hydrophobic bifunctional spacer comprises a hydroxyl group and a carboxylate. In other embodiments, the hydrophobic bifunctional spacer comprises an amine group and a carboxylate. In other embodiments, the hydrophobic bifunctional spacer comprises a thiol group and a carboxylate. Suitable hydrophobic bifunctional spacers comprising a carboxylate and a hydroxyl group or a thiol group are known in the art and include, for example, 8-hydroxyoctanoic acid and 8-mercaptooctanoic acid. In some embodiments, the bifunctional spacer is not a dicarboxylic acid comprising an unbranched, methylene of 1-7 carbon atoms between the carboxylate groups. In some embodiments, the bifunctional spacer is a dicarboxylic acid comprising an unbranched, methylene of 1-7 carbon atoms between the carboxylate groups. The spacer (e.g., amino acid, dipeptide, tripeptide, hydrophilic bifunctional spacer, or hydrophobic bifunctional spacer) in specific embodiments is 3 to 10 atoms (e.g., 6 to 10 atoms, (e.g., 6, 7, 8, 9, or 10 atoms) in length. In more specific embodiments, the spacer is about 3 to 10 atoms (e.g., 6 to 10 atoms) in length and the acyl group is a C₁₂ to C₁₈ fatty acyl group, e.g., C₁₄ fatty acyl group, C₁₆ fatty acyl group, such that the total length of the spacer and acyl group is 14 to 28 atoms, e.g., about 14, 15, 16, 17, 18, 19, 20, 21, 22, 23, 24, 25, 26, 27, or 28 atoms. In some embodiments, the length of the spacer and acyl group is 17 to 28 (e.g., 19 to 26, 19 to 21) atoms. In accordance with certain foregoing embodiments, the bifunctional spacer can be a synthetic or naturally occurring amino acid (including, but not limited to, any of those described herein) comprising an amino acid backbone that is 3 to 10 atoms in length (e.g., 6-amino hexanoic acid, 5-aminovaleric acid, 7-aminoheptanoic acid, and 8-aminooctanoic acid). Alternatively, the spacer can be a dipeptide or tripeptide spacer having a peptide backbone that is 3 to 10 atoms (e.g., 6 to 10 atoms) in length. Each amino acid of the dipeptide or tripeptide spacer can be the same as or different from the other amino acid(s) of the dipeptide or tripeptide and can be independently selected from the group consisting of: naturally-occurring or coded and/or non-coded or non-naturally occurring amino acids, including, for example, any of the D or L isomers of the naturally-occurring amino acids (Ala, Cys, Asp, Glu, Phe, Gly, His, Ile, Lys, Leu, Met, Asn, Pro, Arg, Ser, Thr, Val, Trp, Tyr), or any D or L isomers of the non-naturally occurring or non-coded amino acids selected from the group consisting of: β-alanine (β-Ala), N-α-methyl-alanine (Me-Ala), aminobutyric acid (Abu), γ-aminobutyric acid (7-Abu), aminohexanoic acid (ε-Ahx), aminoisobutyric acid (Aib), aminomethylpyrrole carboxylic acid, aminopiperidinecarboxylic acid, aminoserine (Ams), aminotetrahydropyran-4-carboxylic acid, arginine N-methoxy-N-methyl amide, β-aspartic acid (β-Asp), azetidine carboxylic acid, 3-(2-benzothiazolyl)alanine, α-tert-butylglycine, 2-amino-5-ureido-n-valeric acid (citrulline, Cit), β-Cyclohexylalanine (Cha), acetamidomethyl-cysteine, diaminobutanoic acid (Dab), diaminopropionic acid (Dpr), dihydroxyphenylalanine (DOPA), dimethylthiazolidine (DMTA), γ-Glutamic acid (γ-Glu), homoserine (Hse), hydroxyproline (Hyp), isoleucine N-methoxy-N-methyl amide, methyl-isoleucine (Melle), isonipecotic acid (Isn), methyl-leucine (MeLeu), methyl-lysine, dimethyl-lysine, trimethyl-lysine, methanoproline, methionine-sulfoxide (Met(0)), methionine-sulfone (Met(O₂)), norleucine (Nle), methyl-norleucine (Me-Nle), norvaline (Nva), ornithine (Orn), para-aminobenzoic acid (PABA), penicillamine (Pen), methylphenylalanine (MePhe), 4-Chlorophenylalanine (Phe(4-Cl)), 4-fluorophenylalanine (Phe(4-F)), 4-nitrophenylalanine (Phe(4-NO₂)), 4-cyanophenylalanine ((Phe(4-CN)), phenylglycine (Phg), piperidinylalanine, piperidinylglycine, 3,4-dehydroproline, pyrrolidinylalanine, sarcosine (Sar), selenocysteine (Sec), O-Benzyl-phosphoserine, 4-amino-3-hydroxy-6-methylheptanoic acid (Sta), 4-amino-5-cyclohexyl-3-hydroxypentanoic acid (ACHPA), 4-amino-3-hydroxy-5-phenylpentanoic acid (AHPPA), 1,2,3,4,-tetrahydro-isoquinoline-3-carboxylic acid (Tic), tetrahydropyranglycine, thienylalanine (Thi), O-benzyl-phosphotyrosine, 0-Phosphotyrosine, methoxytyrosine, ethoxytyrosine, O-(bis-dimethylamino-phosphono)-tyrosine, tyrosine sulfate tetrabutylamine, methyl-valine (MeVal), and alkylated 3-mercaptopropionic acid. In some embodiments, the spacer comprises an overall negative charge, e.g., comprises one or two negative-charged amino acids. In some embodiments, the dipeptide is not any of the dipeptides of general structure A-B, wherein A is selected from the group consisting of Gly, Gln, Ala, Arg, Asp, Asn, Ile, Leu, Val, Phe, and Pro, wherein B is selected from the group consisting of Lys, His, Trp. In some embodiments, the dipeptide spacer is selected from the group consisting of: Ala-Ala, β-Ala-β-Ala, Leu-Leu, Pro-Pro, γ-aminobutyric acid-γ-aminobutyric acid, Glu-Glu, and γ-Glu-γ-Glu.

Suitable methods of peptide acylation via amines, hydroxyls, and thiols are known in the art. See, for example, Miller, Biochem Biophys Res Commun 218: 377-382 (1996); Shimohigashi and Stammer, Int J Pept Protein Res 19: 54-62 (1982); and Previero et al., Biochim Biophys Acta 263: 7-13 (1972) (for methods of acylating through a hydroxyl); and San and Silvius, J Pept Res 66: 169-180 (2005) (for methods of acylating through a thiol); Bioconjugate Chem. “Chemical Modifications of Proteins: History and Applications” pages 1, 2-12 (1990); Hashimoto et al., Pharmaceutical Res. “Synthesis of Palmitoyl Derivatives of Insulin and their Biological Activity” Vol. 6, No: 2 pp. 171-176 (1989). The acyl group of the acylated amino acid can be of any size, e.g., any length carbon chain, and can be linear or branched. In some specific embodiments, the acyl group is a C₄ to C₃₀ fatty acid. For example, the acyl group can be any of a C₄ fatty acid, C₆ fatty acid, C₈ fatty acid, C₁₀ fatty acid, C₁₂ fatty acid, C₁₄ fatty acid, C₁₆ fatty acid, C₁₈ fatty acid, C₂₀ fatty acid, C₂₂ fatty acid, C₂₄ fatty acid, C₂₆ fatty acid, C₂₈ fatty acid, or a C₃₀ fatty acid. In some embodiments, the acyl group is a C₈ to C₂₀ fatty acid, e.g., a C₁₄ fatty acid or a C₁₆ fatty acid. In an alternative embodiment, the acyl group is a bile acid. The bile acid can be any suitable bile acid, including, but not limited to, cholic acid, chenodeoxycholic acid, deoxycholic acid, lithocholic acid, taurocholic acid, glycocholic acid, and cholesterol acid. In some embodiments, the peptide comprises an acylated amino acid by acylation of a long chain alkane on the peptide. In specific aspects, the long chain alkane comprises an amine, hydroxyl, or thiol group (e.g., octadecylamine, tetradecanol, and hexadecanethiol) which reacts with a carboxyl group, or activated form thereof, of the peptide. The carboxyl group, or activated form thereof, of the peptide can be part of a side chain of an amino acid (e.g., glutamic acid, aspartic acid) of the peptide or can be part of the analog backbone. In certain embodiments, the peptide is modified to comprise an acyl group by acylation of the long chain alkane by a spacer which is attached to the peptide. In specific aspects, the long chain alkane comprises an amine, hydroxyl, or thiol group which reacts with a carboxyl group, or activated form thereof, of the spacer. Suitable spacers comprising a carboxyl group, or activated form thereof, are described herein and include, for example, bifunctional spacers, e.g., amino acids, dipeptides, tripeptides, hydrophilic bifunctional spacers and hydrophobic bifunctional spacers.

As used herein, the term “activated form” of a carboxyl group refers to a carboxyl group with the general formula R(C═O)X, wherein X is a leaving group and R is the peptide or the spacer. For example, activated forms of a carboxyl groups may include, but are not limited to, acyl chlorides, anhydrides, and esters. In some embodiments, the activated carboxyl group is an ester with a N-hydroxysuccinimide ester (NHS) leaving group.

With regard to these aspects, in which a long chain alkane is acylated by the peptide or the spacer, the long chain alkane may be of any size and can comprise any length of carbon chain. The long chain alkane can be linear or branched. In certain aspects, the long chain alkane is a C₄ to C₃₀ alkane. For example, the long chain alkane can be any of a C₄ alkane, C₆ alkane, C₈ alkane, C₁₀ alkane, C₁₂ alkane, C₁₄ alkane, C₁₆ alkane, C₁₈ alkane, C₂₀ alkane, C₂₂ alkane, C₂₄ alkane, C₂₆ alkane, C₂₈ alkane, or a C₃₀ alkane. In some embodiments, the long chain alkane comprises a C₈ to C₂₀ alkane, e.g., a C₁₄ alkane, C₁₆ alkane, or a C₁₈ alkane.

Also, in some embodiments, an amine, hydroxyl, or thiol group of the peptide is acylated with a cholesterol acid. In a specific embodiment, the peptide is linked to the cholesterol acid through an alkylated des-amino Cys spacer, i.e., an alkylated 3-mercaptopropionic acid spacer. The alkylated des-amino Cys spacer can be, for example, a des-amino-Cys spacer comprising a dodecaethylene glycol moiety.

The peptides described herein can be further modified to comprise a hydrophilic moiety. In some specific embodiments, the hydrophilic moiety can comprise a polyethylene glycol (PEG) chain. The incorporation of a hydrophilic moiety can be accomplished through any suitable means, such as any of the methods described herein. In this regard, the acylated peptide can of any of SEQ ID NO: 1-210, including any of the modifications described herein, in which at least one of the amino acids comprises an acyl group and at least one of the amino acids is covalently bonded to a hydrophilic moiety (e.g., PEG). In some embodiments, the acyl group is attached via a spacer comprising Cys, Lys, Orn, homo-Cys, or Ac-Phe, and the hydrophilic moiety is incorporated at a Cys residue.

Alternatively, the peptides can comprise a spacer, wherein the spacer is both acylated and modified to comprise the hydrophilic moiety. Nonlimiting examples of suitable spacers include a spacer comprising one or more amino acids selected from the group consisting of Cys, Lys, Orn, homo-Cys, and Ac-Phe.

In accordance with some embodiments, the peptide comprises an alkylated amino acid (e.g., a non-coded alkylated amino acid (e.g., an amino acid comprising an alkyl group which is non-native to a naturally-occurring amino acid)). Alkylation can be carried out at any positions within the peptides, including any of the positions described herein as a site for acylation, including but not limited to, any of amino acid positions, at a position within a C-terminal extension, or at the C-terminus, provided that the biological activity is retained. The alkyl group can be covalently linked directly to an amino acid of the peptides, or indirectly to an amino acid of the peptides via a spacer, wherein the spacer is positioned between the amino acid of the peptides and the alkyl group. The peptides may be alkylated at the same amino acid position where a hydrophilic moiety is linked, or at a different amino acid position. In specific aspects, the peptides may be modified to comprise an alkyl group by direct alkylation of an amine, hydroxyl, or thiol of a side chain of an amino acid of the peptides. In this regard, the alkylated peptides can comprise an amino acid sequence with at least one of the amino acids modified to any amino acid comprising a side chain amine, hydroxyl, or thiol. In yet other embodiments, the amino acid comprising a side chain amine, hydroxyl, or thiol is a disubstituted amino acid. In some embodiments, the alkylated peptide comprises a spacer between the peptide and the alkyl group. In some embodiments, the peptide is covalently bound to the spacer, which is covalently bound to the alkyl group. In some exemplary embodiments, the peptide is modified to comprise an alkyl group by alkylation of an amine, hydroxyl, or thiol of a spacer, which spacer is attached to a side chain of an amino acid. The amino acid to which the spacer is attached can be any amino acid comprising a moiety which permits linkage to the spacer. For example, an amino acid comprising a side chain NH₂, —OH, or —COOH (e.g., Lys, Orn, Ser, Asp, or Glu) is suitable. In some embodiments, the spacer is an amino acid comprising a side chain amine, hydroxyl, or thiol or a dipeptide or tripeptide comprising an amino acid comprising a side chain amine, hydroxyl, or thiol. When alkylation occurs through an amine group of a spacer, the alkylation can occur through the alpha amine of an amino acid or a side chain amine. In the instance in which the alpha amine is alkylated, the amino acid of the spacer can be any amino acid. For example, the amino acid of the spacer can be a hydrophobic amino acid, e.g., Gly, Ala, Val, Leu, Ile, Trp, Met, Phe, Tyr, 6-amino hexanoic acid, 5-aminovaleric acid, 7-aminoheptanoic acid, and 8-aminooctanoic acid. Alternatively, the amino acid of the spacer can be an acidic residue, e.g., Asp and Glu, provided that the alkylation occurs on the alpha amine of the acidic residue. In the instance in which the side chain amine of the amino acid of the spacer is alkylated, the amino acid of the spacer is an amino acid comprising a side chain amine, e.g., an amino acid of Formula I (e.g., Lys or Orn). In this instance, it is possible for both the alpha amine and the side chain amine of the amino acid of the spacer to be alkylated, such that the peptide is dialkylated. Embodiments include such dialkylated molecules. When alkylation occurs through a hydroxyl group of a spacer, the amino acid can be Ser. When alkylation occurs through a thiol group of spacer, the amino acid can be Cys. In some embodiments, the spacer is a hydrophilic bifunctional spacer. In certain embodiments, the hydrophilic bifunctional spacer comprises two or more reactive groups, e.g., an amine, a hydroxyl, a thiol, and a carboxyl group or any combinations thereof. In certain embodiments, the hydrophilic bifunctional spacer comprises a hydroxyl group and a carboxylate. In other embodiments, the hydrophilic bifunctional spacer comprises an amine group and a carboxylate. In other embodiments, the hydrophilic bifunctional spacer comprises a thiol group and a carboxylate. In a specific embodiment, the spacer comprises an amino poly(alkyloxy)carboxylate. In this regard, the spacer can comprise, for example, NH₂(CH₂CH₂O)ₙ(CH₂)ₘCOOH, wherein m is any integer from 1 to 6 and n is any integer from 2 to 12, such as, e.g., 8-amino-3,6-dioxaoctanoic acid, which is commercially available from Peptides International, Inc. (Louisville, Ky.). Suitable hydrophobic bifunctional spacers comprising a carboxylate and a hydroxyl group or a thiol group are known in the art and include, for example, 8-hydroxyoctanoic acid and 8-mercaptooctanoic acid. The spacer (e.g., amino acid, dipeptide, tripeptide, hydrophilic bifunctional spacer, or hydrophobic bifunctional spacer) in specific embodiments is 3 to 10 atoms (e.g., 6 to 10 atoms, (e.g., 6, 7, 8, 9, or 10 atoms)) in length. In more specific embodiments, the spacer is about 3 to 10 atoms (e.g., 6 to 10 atoms) in length and the alkyl is a C₁₂ to C₁₈ alkyl group, e.g., C₁₄ alkyl group, C₁₆ alkyl group, such that the total length of the spacer and alkyl group is 14 to 28 atoms, e.g., about 14, 15, 16, 17, 18, 19, 20, 21, 22, 23, 24, 25, 26, 27, or 28 atoms. In some embodiments, the length of the spacer and alkyl is 17 to 28 (e.g., 19 to 26, 19 to 21) atoms. In accordance with certain foregoing embodiments, the bifunctional spacer can be a synthetic or non-naturally occurring or non-coded amino acid comprising an amino acid backbone that is 3 to 10 atoms in length (e.g., 6-amino hexanoic acid, 5-aminovaleric acid, 7-aminoheptanoic acid, and 8-aminooctanoic acid). Alternatively, the spacer can be a dipeptide or tripeptide spacer having a peptide backbone that is 3 to 10 atoms (e.g., 6 to 10 atoms) in length. The dipeptide or tripeptide spacer can be composed of naturally-occurring or coded and/or non-coded or non-naturally occurring amino acids, including, for example, any of the amino acids taught herein. In some embodiments, the spacer comprises an overall negative charge, e.g., comprises one or two negative-charged amino acids. In some embodiments, the dipeptide spacer is selected from the group consisting of: Ala-Ala, β-Ala-β-Ala, Leu-Leu, Pro-Pro, γ-aminobutyric acid-γ-aminobutyric acid, and γ-Glu-γ-Glu. Suitable methods of peptide alkylation via amines, hydroxyls, and thiols are known in the art. For example, a Williamson ether synthesis can be used to form an ether linkage between a hydroxyl group of the peptides and the alkyl group. Also, a nucleophilic substitution reaction of the peptide with an alkyl halide can result in any of an ether, thioether, or amino linkage. The alkyl group of the alkylated peptides can be of any size, e.g., any length carbon chain, and can be linear or branched. In some embodiments, the alkyl group is a C₄ to C₃₀ alkyl. For example, the alkyl group can be any of a C₄ alkyl, C₆ alkyl, C₈ alkyl, C₁₀ alkyl, C₁₂ alkyl, C₁₄ alkyl, C₁₆ alkyl, C₁₈ alkyl, C₂₀ alkyl, C₂₂ alkyl, C₂₄ alkyl, C₂₆ alkyl, C₂₈ alkyl, or a C₃₀ alkyl. In some embodiments, the alkyl group is a C₈ to C₂₀ alkyl, e.g., a C₁₄ alkyl or a C₁₆ alkyl. In some embodiments of the disclosure, the peptide comprises an alkylated amino acid by reacting a nucleophilic, long chain alkane with the peptide, wherein the peptide comprises a leaving group suitable for nucleophilic substitution. In specific aspects, the nucleophilic group of the long chain alkane comprises an amine, hydroxyl, or thiol group (e.g., octadecylamine, tetradecanol, and hexadecanethiol). The leaving group of the peptide can be part of a side chain of an amino acid or can be part of the peptide backbone. Suitable leaving groups include, for example, N-hydroxysuccinimide, halogens, and sulfonate esters. In certain embodiments, the peptide is modified to comprise an alkyl group by reacting the nucleophilic, long chain alkane with a spacer which is attached to the peptide, wherein the spacer comprises the leaving group. In specific aspects, the long chain alkane comprises an amine, hydroxyl, or thiol group. In certain embodiments, the spacer comprising the leaving group can be any spacer discussed herein, e.g., amino acids, dipeptides, tripeptides, hydrophilic bifunctional spacers and hydrophobic bifunctional spacers further comprising a suitable leaving group. With regard to these aspects of the disclosure, in which a long chain alkane is alkylated by the peptides or the spacer, the long chain alkane may be of any size and can comprise any length of carbon chain. The long chain alkane can be linear or branched. In certain aspects, the long chain alkane is a C₄ to C₃₀ alkane. For example, the long chain alkane can be any of a C₄ alkane, C₆ alkane, C₈ alkane, Cm alkane, C₁₂ alkane, C₁₄ alkane, C₁₆ alkane, C₁₈ alkane, C₂₀ alkane, C₂₂ alkane, C₂₄ alkane, C₂₆ alkane, C₂₈ alkane, or a C₃₀ alkane. In some embodiments, the long chain alkane comprises a C₈ to C₂₀ alkane, e.g., a C₁₄ alkane, C₁₆ alkane, or a C₁₈ alkane. Also, in some embodiments, alkylation can occur between the peptides and a cholesterol moiety. For example, the hydroxyl group of cholesterol can displace a leaving group on the long chain alkane to form a cholesterol-peptides product. The alkylated peptides described herein can be further modified to comprise a hydrophilic moiety. In some specific embodiments the hydrophilic moiety can comprise a polyethylene glycol (PEG) chain. The incorporation of a hydrophilic moiety can be accomplished through any suitable means, such as any of the methods described herein. Alternatively, the alkylated peptides can comprise a spacer, wherein the spacer is both alkylated and modified to comprise the hydrophilic moiety. Nonlimiting examples of suitable spacers include a spacer comprising one or more amino acids selected from the group consisting of Cys, Lys, Orn, homo-Cys, and Ac-Phe.

In some embodiments, the peptide comprises at position 1 or 2, or at both positions 1 and 2, an amino acid which achieves resistance of the peptides to peptidase cleavage. In some embodiments, the peptide comprises at position 1 an amino acid selected from the group consisting of: D-histidine, desaminohistidine, hydroxyl-histidine, acetyl-histidine, homo-histidine, N-methyl histidine, alpha-methyl histidine, imidazole acetic acid, or alpha, alpha-dimethyl imidazole acetic acid (DMIA). In some embodiments, the peptide comprises at position 2 an amino acid selected from the group consisting of: D-serine, D-alanine, valine, glycine, N-methyl serine, N-methyl alanine, or alpha, aminoisobutyric acid. In some embodiments, the peptide comprises at position 2 an amino acid which achieves resistance of the peptide to peptidases and the amino acid which achieves resistance of the peptide to peptidases is not D-serine. In some embodiments, this covalent bond is an intramolecular bridge other than a lactam bridge. For example, suitable covalent bonding methods include any one or more of olefin metathesis, lanthionine-based cyclization, disulfide bridge or modified sulfur-containing bridge formation, the use of α,ω-diaminoalkane tethers, the formation of metal-atom bridges, and other means of peptide cyclization.

In some embodiments, the peptide is modified by amino acid substitutions and/or additions that introduce a charged amino acid into the C-terminal portion of the analog. In some embodiments, such modifications enhance stability and solubility. As used herein the term “charged amino acid” or “charged residue” refers to an amino acid that comprises a side chain that is negative-charged (i.e., de-protonated) or positive-charged (i.e., protonated) in aqueous solution at physiological pH. In some aspects, these amino acid substitutions and/or additions that introduce a charged amino acid modifications may be at a C-terminal position. In some embodiments, one, two or three (and in some instances, more than three) charged amino acids may be introduced at the C-terminal position. In exemplary embodiments, one, two or all of the charged amino acids may be negative-charged. The negative-charged amino acid in some embodiments is aspartic acid, glutamic acid, cysteic acid, homocysteic acid, or homoglutamic acid. In some aspects, these modifications increase solubility.

In accordance with some embodiments, the peptides disclosed herein may be modified by truncation of the C-terminus by one or two amino acid residues. In this regard, the peptides can comprise the sequences (SEQ ID NO: 1-210), optionally with any of the additional modifications described herein.

In some embodiments, the peptide comprises a modified SEQ ID NO: 1-210 in which the carboxylic acid of the C-terminal amino acid is replaced with a charge-neutral group, such as an amide or ester. Accordingly, in some embodiments, the peptide is an amidated peptide, such that the C-terminal residue comprises an amide in place of the alpha carboxylate of an amino acid. As used herein a general reference to a peptide or analog is intended to encompass peptides that have a modified amino terminus, a modified carboxy terminus, or modifications of both amino and carboxy termini. For example, an amino acid chain composing an amide group in place of the terminal carboxylic acid is intended to be encompassed by an amino acid sequence designating the standard amino acids.

In accordance with some embodiments, the peptides disclosed herein may be modified by conjugation on at least one amino acid residue. In this regard, the peptides can comprise the sequences (SEQ ID NO: 1-210), optionally with any of the additional conjugations described herein.

The disclosure further provides conjugates comprising one or more of the peptides described herein conjugated to a heterologous moiety. As used herein, the term “heterologous moiety” is synonymous with the term “conjugate moiety” and refers to any molecule (chemical or biochemical, naturally-occurring or non-coded) which is different from the peptides described herein. Exemplary conjugate moieties that can be linked to any of the analogs described herein include but are not limited to a heterologous peptide or polypeptide (including for example, a plasma protein), a targeting agent, an immunoglobulin or portion thereof (e.g., variable region, CDR, or Fc region), a diagnostic label such as a radioisotope, fluorophore or enzymatic label, a polymer including water soluble polymers, or other therapeutic or diagnostic agents. In some embodiments, a conjugate is provided comprising a peptide and a plasma protein, wherein the plasma protein is selected from the group consisting of albumin, transferin, fibrinogen and globulins. In some embodiments, the plasma protein moiety of the conjugate is albumin or transferin.

The conjugate in some embodiments comprises one or more of the peptides described herein and one or more of: a different peptide (which is distinct from the peptides described herein), a polypeptide, a nucleic acid molecule, an antibody or fragment thereof, a polymer, a quantum dot, a small molecule, a toxin, a diagnostic agent, a carbohydrate, an amino acid. In some embodiments, the heterologous moiety is a polymer. In some embodiments, the polymer is selected from the group consisting of: polyamides, polycarbonates, polyalkylenes and derivatives thereof including, polyalkylene glycols, polyalkylene oxides, polyalkylene terepthalates, polymers of acrylic and methacrylic esters, including poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), polyvinyl polymers including polyvinyl alcohols, polyvinyl ethers, polyvinyl esters, polyvinyl halides, poly(vinyl acetate), and polyvinylpyrrolidone, polyglycolides, polysiloxanes, polyurethanes and co-polymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt, polypropylene, polyethylenes including poly(ethylene glycol), poly(ethylene oxide), and poly(ethylene terephthalate), and polystyrene. In some aspects, the polymer is a biodegradable polymer, including a synthetic biodegradable polymer (e.g., polymers of lactic acid and glycolic acid, polyanhydrides, poly(ortho)esters, polyurethanes, poly(butic acid), poly(valeric acid), and poly(lactide-cocaprolactone)), and a natural biodegradable polymer (e.g., alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins (e.g., zein and other prolamines and hydrophobic proteins)), as well as any copolymer or mixture thereof. In general, these materials degrade either by enzymatic hydrolysis or exposure to water in vivo, by surface or bulk erosion. In some aspects, the polymer is a bioadhesive polymer, such as a bioerodible hydrogel described by H. S. Sawhney, C. P. Pathak and J. A. Hubbell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In some embodiments, the polymer is a water-soluble polymer or a hydrophilic polymer. Hydrophilic polymers are further described herein under “Hydrophilic Moieties.” Suitable water-soluble polymers are known in the art and include, for example, polyvinylpyrrolidone, hydroxypropyl cellulose (HPC; Klucel), hydroxypropyl methylcellulose (HPMC; Methocel), nitrocellulose, hydroxypropyl ethylcellulose, hydroxypropyl butylcellulose, hydroxypropyl pentylcellulose, methyl cellulose, ethylcellulose (Ethocel), hydroxyethyl cellulose, various alkyl celluloses and hydroxyalkyl celluloses, various cellulose ethers, cellulose acetate, carboxymethyl cellulose, sodium carboxymethyl cellulose, calcium carboxymethyl cellulose, vinyl acetate/crotonic acid copolymers, poly-hydroxyalkyl methacrylate, hydroxymethyl methacrylate, methacrylic acid copolymers, polymethacrylic acid, polymethylmethacrylate, maleic anhydride/methyl vinyl ether copolymers, poly vinyl alcohol, sodium and calcium polyacrylic acid, polyacrylic acid, acidic carboxy polymers, carboxypolymethylene, carboxyvinyl polymers, polyoxyethylene polyoxypropylene copolymer, polymethylvinylether co-maleic anhydride, carboxymethylamide, potassium methacrylate divinylbenzene co-polymer, polyoxyethyleneglycols, polyethylene oxide, and derivatives, salts, and combinations thereof. In specific embodiments, the polymer is a polyalkylene glycol, including, for example, polyethylene glycol (PEG).

In some embodiments, the heterologous moiety is a carbohydrate. In some embodiments, the carbohydrate is a monosaccharide (e.g., glucose, galactose, fructose), a disaccharide (e.g., sucrose, lactose, maltose), an oligosaccharide (e.g., raffinose, stachyose), a polysaccharide (a starch, amylase, amylopectin, cellulose, chitin, callose, laminarin, xylan, mannan, fucoidan, galactomannan.

In some embodiments, the heterologous moiety is a lipid. The lipid, in some embodiments, is a fatty acid, eicosanoid, prostaglandin, leukotriene, thromboxane, N-acyl ethanolamine), glycerolipid (e.g., mono-, di-, tri-substituted glycerols), glycerophospholipid (e.g., phosphatidylcholine, phosphatidylinositol, phosphatidylethanolamine, phosphatidylserine), sphingolipid (e.g., sphingosine, ceramide), sterol lipid (e.g., steroid, cholesterol), prenol lipid, saccharolipid, or a polyketide, oil, wax, cholesterol, sterol, fat-soluble vitamin, monoglyceride, diglyceride, triglyceride, a phospholipid.

In some embodiments, the heterologous moiety is attached via non-covalent or covalent bonding to the peptide of the present disclosure. In certain aspects, the heterologous moiety is attached to the peptide of the present disclosure via a linker. Linkage can be accomplished by covalent chemical bonds, physical forces such electrostatic, hydrogen, ionic, van der Waals, or hydrophobic or hydrophilic interactions. A variety of non-covalent coupling systems may be used, including biotin-avidin, ligand/receptor, enzyme/substrate, nucleic acid/nucleic acid binding protein, lipid/lipid binding protein, cellular adhesion molecule partners; or any binding partners or fragments thereof which have affinity for each other. The peptide in some embodiments is linked to conjugate moieties via direct covalent linkage by reacting targeted amino acid residues of the analog with an organic derivatizing agent that is capable of reacting with selected side chains or the N- or C-terminal residues of these targeted amino acids. Reactive groups on the analog or conjugate moiety include, e.g., an aldehyde, amino, ester, thiol, α-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art. Alternatively, the conjugate moieties can be linked to the analog indirectly through intermediate carriers, such as polysaccharide or polypeptide carriers. Examples of polysaccharide carriers include aminodextran. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier. Cysteinyl residues are most commonly reacted with α-haloacetates (and corresponding amines), such as chloroacetic acid, chloroacetamide to give carboxymethyl or carboxyamidomethyl derivatives. Cysteinyl residues also may be derivatized by reaction with bromotrifluoroacetone, alpha-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues may be derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0. Lysinyl and amino-terminal residues may be reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate. Arginyl residues may be modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group. The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form 0-acetyl tyrosyl species and 3-nitro derivatives, respectively. Carboxyl side groups (aspartyl or glutamyl) may be selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues may be converted to asparaginyl and glutaminyl residues by reaction with ammonium ions. Other modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)), deamidation of asparagine or glutamine, acetylation of the N-terminal amine, and/or amidation or esterification of the C-terminal carboxylic acid group. Another type of covalent modification involves chemically or enzymatically coupling glycosides to the peptide. Sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of tyrosine, or tryptophan, or (f) the amide group of glutamine. These methods are described in WO87/05330 published 11 Sep. 1987, and in Aplin and Wriston, CRC Crit. Rev. Biochem., pp. 259-306 (1981). In some embodiments, the peptide is conjugated to a heterologous moiety via covalent linkage between a side chain of an amino acid of the peptides and the heterologous moiety. In some aspects, the amino acid covalently linked to a heterologous moiety (e.g., the amino acid comprising a heterologous moiety) is a Cys, Lys, Orn, homo-Cys, or Ac-Phe, and the side chain of the amino acid is covalently bonded to a heterologous moiety. In some embodiments, the conjugate comprises a linker that joins the peptide to the heterologous moiety. In some aspects, the linker comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms may be all carbon atoms. In some embodiments, the chain atoms in the backbone of the linker may be selected from the group consisting of C, O, N, and S. Chain atoms and linkers may be selected according to their expected solubility (hydrophilicity) so as to provide a more soluble conjugate. In some embodiments, the linker provides a functional group that is subject to cleavage by an enzyme or other catalyst or hydrolytic conditions found in the target tissue or organ or cell. In some embodiments, the length of the linker is long enough to reduce the potential for steric hindrance. If the linker is a covalent bond or a peptidyl bond and the conjugate is a polypeptide, the entire conjugate can be a fusion protein. Such peptidyl linkers may be any length. Exemplary linkers may be from about 1 to 50 amino acids in length, 5 to 50, 3 to 5, 5 to 10, 5 to 15, or 10 to 30 amino acids in length. Such fusion proteins may alternatively be produced by recombinant genetic engineering methods known to one of ordinary skill in the art.

As noted above, in some embodiments, the peptides may be conjugated, e.g., fused to an immunoglobulin or portion thereof (e.g., variable region, CDR, or Fc region). Known types of immunoglobulins (Ig) include IgG, IgA, IgE, IgD or IgM. The Fc region is a C-terminal region of an Ig heavy chain, which is responsible for binding to Fc receptors that carry out activities such as recycling (which results in prolonged half-life), antibody dependent cell-mediated cytotoxicity (ADCC), and complement dependent cytotoxicity (CDC). For example, according to some definitions the human IgG heavy chain Fc region stretches from Cys226 to the C-terminus of the heavy chain. The “hinge region” generally extends from Glu216 to Pro230 of human IgG1 (hinge regions of other IgG isotypes may be aligned with the IgG1 sequence by aligning the cysteines involved in cysteine bonding). The Fc region of an IgG includes two constant domains, CH2 and CH3. The CH2 domain of a human IgG Fc region usually extends from amino acids 231 to amino acid 341. The CH3 domain of a human IgG Fc region usually extends from amino acids 342 to 447. References made to amino acid numbering of immunoglobulins or immunoglobulin fragments, or regions, are all based on Kabat et al. 1991, Sequences of Proteins of Immunological Interest, U.S. Department of Public Health, Bethesda, Md. In related embodiments, the Fc region may comprise one or more native or modified constant regions from an immunoglobulin heavy chain, other than CH1, for example, the CH2 and CH3 regions of IgG and IgA, or the CH3 and CH4 regions of IgE. Suitable conjugate moieties include portions of immunoglobulin sequence that include the FcRn binding site. FcRn, a salvage receptor, is responsible for recycling immunoglobulins and returning them to circulation in blood. The region of the Fc portion of IgG that binds to the FcRn receptor has been described based on X-ray crystallography (Burmeister et al. 1994, Nature 372:379). The major contact area of the Fc with the FcRn is near the junction of the CH2 and CH3 domains. Fc-FcRn contacts are all within a single Ig heavy chain. The major contact sites include amino acid residues 248, 250-257, 272, 285, 288, 290-291, 308-311, and 314 of the CH2 domain and amino acid residues 385-387, 428, and 433-436 of the CH3 domain. Some conjugate moieties may or may not include FcγR binding site(s). FcγR are responsible for ADCC and CDC. Examples of positions within the Fc region that make a direct contact with FcγR are amino acids 234-239 (lower hinge region), amino acids 265-269 (B/C loop), amino acids 297-299 (C′/E loop), and amino acids 327-332 (F/G) loop (Sondermann et al., Nature 406: 267-273, 2000). The lower hinge region of IgE has also been implicated in the FcRI binding (Henry, et al., Biochemistry 36, 15568-15578, 1997). Residues involved in IgA receptor binding are described in Lewis et al., (J Immunol. 175:6694-701, 2005). Amino acid residues involved in IgE receptor binding are described in Sayers et al. (J Biol Chem. 279(34):35320-5, 2004) Amino acid modifications may be made to the Fc region of an immunoglobulin. Such variant Fc regions comprise at least one amino acid modification in the CH3 domain of the Fc region (residues 342-447) and/or at least one amino acid modification in the CH2 domain of the Fc region (residues 231-341). Mutations believed to impart an increased affinity for FcRn include T256A, T307A, E380A, and N434A (Shields et al. 2001, J. Biol. Chem. 276:6591). Other mutations may reduce binding of the Fc region to FcγRI, FcγRIIA, FcγRIIB, and/or FcγRIIIA without significantly reducing affinity for FcRn. For example, substitution of the Asn at position 297 of the Fc region with Ala or another amino acid removes a highly conserved N-glycosylation site and may result in reduced immunogenicity with concomitant prolonged half-life of the Fc region, as well as reduced binding to FcγRs (Routledge et al. 1995, Transplantation 60:847; Friend et al. 1999, Transplantation 68:1632; Shields et al. 1995, J. Biol. Chem. 276:6591). Amino acid modifications at positions 233-236 of IgG1 have been made that reduce binding to FcγRs (Ward and Ghetie 1995, Therapeutic Immunology 2:77 and Armour et al. 1999, Eur. J. Immunol. 29:2613). Some exemplary amino acid substitutions are described in U.S. Pat. Nos. 7,355,008 and 7,381,408, each incorporated by reference herein in its entirety. In certain embodiments, a peptide described herein is inserted into a loop region within the immunoglobulin molecule. In other embodiments, a peptide described herein replaces one or more amino acids of a loop region within the immunoglobulin molecule.

The peptides described herein can be further modified to improve its solubility and stability in aqueous solutions at physiological pH, while retaining the biological activity. Hydrophilic moieties such as PEG groups can be attached to the analogs under any suitable conditions used to react a protein with an activated polymer molecule. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the target compound (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., alpha-iodo acetic acid, alpha-bromoacetic acid, alpha-chloroacetic acid). If attached to the analog by reductive alkylation, the polymer selected should have a single reactive aldehyde so that the degree of polymerization is controlled. See, for example, Kinstler et al., Adv. Drug. Delivery Rev. 54: 477-485 (2002); Roberts et al., Adv. Drug Delivery Rev. 54: 459-476 (2002); and Zalipsky et al., Adv. Drug Delivery Rev. 16: 157-182 (1995). In specific aspects, an amino acid residue of the peptides having a thiol is modified with a hydrophilic moiety such as PEG. In some embodiments, the thiol is modified with maleimide-activated PEG in a Michael addition reaction to result in a PEGylated analog comprising a thioether linkage. In some embodiments, the thiol is modified with a haloacetyl-activated PEG in a nucleophilic substitution reaction to result in a PEGylated analog comprising a thioether linkage. Suitable hydrophilic moieties include polyethylene glycol (PEG), polypropylene glycol, polyoxyethylated polyols (e.g., POG), polyoxyethylated sorbitol, polyoxyethylated glucose, polyoxyethylated glycerol (POG), polyoxyalkylenes, polyethylene glycol propionaldehyde, copolymers of ethylene glycol/propylene glycol, monomethoxy-polyethylene glycol, mono-(C1-C10) alkoxy- or aryloxy-polyethylene glycol, carboxymethylcellulose, polyacetals, polyvinyl alcohol (PVA), polyvinyl pyrrolidone, poly-1,3-dioxolane, poly-1,3,6-trioxane, ethylene/maleic anhydride copolymer, poly (.beta-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, colonic acids or other polysaccharide polymers, Ficoll or dextran and mixtures thereof. Dextrans are polysaccharide polymers of glucose subunits, predominantly linked by al-6 linkages. Dextran is available in many molecular weight ranges, e.g., about 1 kD to about 100 kD, or from about 5, 10, 15 or 20 kD to about 20, 30, 40, 50, 60, 70, 80 or 90 kD. Linear or branched polymers are contemplated. Resulting preparations of conjugates may be essentially monodisperse or polydisperse, and may have about 0.5, 0.7, 1, 1.2, 1.5 or 2 polymer moieties per analog.

In some embodiments, the peptide is conjugated to a hydrophilic moiety via covalent linkage between a side chain of an amino acid of the peptide and the hydrophilic moiety. In some embodiments, the peptide is conjugated to a hydrophilic moiety via the side chain of an amino acid, a position within a C-terminal extension, or the C-terminal amino acid, or a combination of these positions. In some aspects, the amino acid covalently linked to a hydrophilic moiety (e.g., the amino acid comprising a hydrophilic moiety) is a Cys, Lys, Orn, homo-Cys, or Ac-Phe, and the side chain of the amino acid is covalently bonded to a hydrophilic moiety (e.g., PEG). In some embodiments, the conjugate of the present disclosure comprises the peptide fused to an accessory analog which is capable of forming an extended conformation similar to chemical PEG (e.g., a recombinant PEG (rPEG) molecule), such as those described in International Patent Application Publication No. WO2009/023270 and U.S. Patent Application Publication No. US20080286808. The rPEG molecule in some aspects is a polypeptide comprising one or more of glycine, serine, glutamic acid, aspartic acid, alanine, or proline. In some aspects, the rPEG is a homopolymer, e.g., poly-glycine, poly-serine, poly-glutamic acid, poly-aspartic acid, poly-alanine, or poly-proline. In other embodiments, the rPEG comprises two types of amino acids repeated, e.g., poly(Gly-Ser), poly(Gly-Glu), poly(Gly-Ala), poly(Gly-Asp), poly(Gly-Pro), poly(Ser-Glu), etc. In some aspects, the rPEG comprises three different types of amino acids, e.g., poly(Gly-Ser-Glu). In specific aspects, the rPEG increases the half-life of the peptide. In some aspects, the rPEG comprises a net positive or net negative charge. The rPEG in some aspects lacks secondary structure. In some embodiments, the rPEG is greater than or equal to 10 amino acids in length and in some embodiments is about 40 to about 50 amino acids in length. The accessory peptide in some aspects is fused to the N- or C-terminus of the peptide of the present disclosure through a peptide bond or a proteinase cleavage site, or is inserted into the loops of the peptide of the present disclosure. The rPEG in some aspects comprises an affinity tag or is linked to a PEG that is greater than 5 kDa. In some embodiments, the rPEG confers the peptide of the present disclosure with an increased hydrodynamic radius, serum half-life, protease resistance, or solubility and in some aspects confers the analog with decreased immunogenicity.

The peptides comprising the sequences (SEQ ID NO: 1-210), optionally with any of the conjugations described herein are contemplated as an embodiment.

The disclosure further provides multimers or dimers of the peptides disclosed herein, including homo- or hetero-multimers or homo- or hetero-dimers. Two or more of the analogs can be linked together using standard linking agents and procedures known to those skilled in the art. For example, dimers can be formed between two peptides through the use of bifunctional thiol crosslinkers and bi-functional amine crosslinkers, particularly for the analogs that have been substituted with cysteine, lysine ornithine, homocysteine or acetyl phenylalanine residues. The dimer can be a homodimer or alternatively can be a heterodimer. In certain embodiments, the linker connecting the two (or more) analogs is PEG, e.g., a 5 kDa PEG, 20 kDa PEG. In some embodiments, the linker is a disulfide bond. For example, each monomer of the dimer may comprise a Cys residue (e.g., a terminal or internally positioned Cys) and the sulfur atom of each Cys residue participates in the formation of the disulfide bond. In some aspects, the monomers may be connected via terminal amino acids (e.g., N-terminal or C-terminal), via internal amino acids, or via a terminal amino acid of at least one monomer and an internal amino acid of at least one other monomer. In specific aspects, the monomers are not connected via an N-terminal amino acid. In some aspects, the monomers of the multimer may be attached together in a “tail-to-tail” orientation in which the C-terminal amino acids of each monomer may be attached together.

Peptides disclosed herein may be made in a variety of ways. Suitable methods of de novo synthesizing peptides are described in, for example, Merrifield, J. Am. Chem. Soc, 85, 2149 (1963); Davis et al., Biochem. Intl., 10, 394-414 (1985); Larsen et al., J. Am. Chem. Soc, 115, 6247 (1993); Smith et al., J. Peptide Protein Res., 44, 183 (1994); O'Donnell et al., J. Am. Chem. Soc, 118, 6070 (1996); Stewart and Young, Solid Phase Peptide Synthesis, Freeman (1969); Finn et al., The Proteins, 3 ed., vol. 2, pp. 105-253 (1976); Erickson et al., The Proteins, 3^{rd} ed., vol. 2, pp. 257-527 (1976); and Chan et al., Fmoc Solid Phase Peptide Synthesis, Oxford University Press, Oxford, United Kingdom, 2005. The disclosure contemplates synthetic peptides. Methods of making the peptides are themselves embodiments of the invention.

Alternatively, the peptide can be expressed recombinantly by introducing a nucleic acid encoding a peptide into host cells, which may be cultured to express the peptide using standard recombinant methods. See, for instance, Sambrook et al., Molecular Cloning: A Laboratory Manual. 3rd ed., Cold Spring Harbor Press, Cold Spring Harbor, N.Y. 2001; and Ausubel et al., Current Protocols in Molecular Biology, Greene Publishing Associates and John Wiley & Sons, N.Y., 1994. Such peptides may be purified from the culture media or cell pellets.

In some embodiments, the peptides of the disclosure can be isolated. In some embodiments, the peptides of the disclosure may be purified. It is recognized that “purity” is a relative term, and not to be necessarily construed as absolute purity or absolute enrichment or absolute selection. In some aspects, the purity is at least or about 50%, is at least or about 60%, at least or about 70%, at least or about 80%, or at least or about 90% (e.g., at least or about 91%, at least or about 92%, at least or about 93%, at least or about 94%, at least or about 95%, at least or about 96%, at least or about 97%, at least or about 98%, at least or about 99% or is approximately 100%.

In some embodiments, the peptides described herein can be commercially synthesized by companies, such as Genscript (Piscataway, N.J.), New England Peptide (Gardner, Mass.), and CPC Scientific (Sunnyvale, Calif.), Peptide Technologies Corp. (Gaithersburg, Md.), and Multiple Peptide Systems (San Diego, Calif.). In this respect, the peptides can be synthetic, recombinant, isolated, and/or purified.

The peptides of the present disclosure can be provided in accordance with one embodiment as part of a kit. Accordingly, in some embodiments, a kit for administering a peptide, to a patient in need thereof is provided wherein the kit comprises a peptide as described herein.

In one embodiment, the kit is provided with a device for administering the composition to a patient, e.g., syringe needle, pen device, jet injector or another needle-free injector. The kit may alternatively or in addition include one or more containers, e.g., vials, tubes, bottles, single or multi-chambered pre-filled syringes, cartridges, infusion pumps (external or implantable), jet injectors, pre-filled pen devices and the like, optionally containing the peptide in a lyophilized form or in an aqueous solution. The kits in some embodiments comprise instructions for use. In accordance with one embodiment the device of the kit is an aerosol dispensing device, wherein the composition is prepackaged within the aerosol device. In another embodiment the kit comprises a syringe and a needle, and in one embodiment the sterile composition is prepackaged within the syringe.

A further embodiment includes a process of treating a disease comprising one or more of prescribing, selling or advertising to sell, purchasing, instructing to self-administer, or administering a peptide described herein, wherein the peptide has been approved by a regulatory agency for the treatment of a condition, to a subject in need of treatment.

A further embodiment includes a method of supplying a peptide for treating a disease, said method comprises reimbursing a physician, a formulary, a patient or an insurance company for the sale of said peptide.

### Definitions

The terms “peptide” refers to a molecule comprising two or more amino acid residues joined to each other by peptide bonds. These terms encompass, e.g., native and artificial proteins, protein fragments and polypeptide analogs (such as muteins, variants, and fusion proteins) of a protein sequence as well as post-translationally, or otherwise covalently or non-covalently, modified peptides. A peptide may be monomeric or polymeric. In certain embodiments, “peptides” are chains of amino acids whose alpha carbons may be linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free α-amino group on an amino acid at the amino terminal of a peptide or to the α-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether as opposed to an amide bond.

The term “therapeutic peptide” refers to peptides or fragments or variants thereof, having one or more therapeutic and/or biological activities.

The term “analog” as used herein describes a peptide comprising one or more amino acid modifications, such as but not limited to substitution and/or one or more deletion and/or one or more addition of any one of the amino acid residues for any natural or unnatural amino acid, synthetic amino acids or peptidomimetics and/or the attachment of a side chain to any one of the natural or unnatural amino acids, synthetic amino acids or peptidomimetics at any available position. The addition or deletion of amino acid residues can take place at the N-terminal of the peptide and/or at the C-terminal of the peptide.

In some embodiments, the analog has 1, 2, 3, 4, or 5 such modifications. In some embodiments, the analog retains biological activity of the original peptide. In some embodiments, the analog is a competitive or non-competitive inhibitor of the original peptide.

Peptide sequences are indicated using standard one- or three-letter abbreviations. Unless otherwise indicated, peptide sequences have their amino termini at the left and their carboxy termini at the right, A particular section of a peptide can be designated by amino acid residue number such as amino acids 3 to 6, or by the actual residue at that site such as Met3 to Gly6. A particular peptide sequence also can be described by explaining how it differs from a reference sequence.

When used herein the term “natural amino acid” is an amino acid (with the usual three letter codes & one letter codes in parenthesis) selected from the group consisting of: Glycine (Gly & G), proline (Pro & P), alanine (Ala & A), valine (Val & V), leucine (Leu & L), isoleucine (Ile & I), methionine (Met & M), cysteine (Cys & C), phenylalanine (Phe & F), tyrosine (Tyr & Y), tryptophan (Trp & W), histidine (His & H), lysine (Lys & K), arginine (Arg & R), glutamine (Gin & Q), asparagine (Asn & N), glutamic acid (Glu & E), aspartic acid (Asp & D), serine (Ser & S) and threonine (Thr & T). If anywhere herein, reference is made to a peptide, analog or derivative or peptides comprising or not comprising G, P, A, V, L, I, M, C, F, Y, H, K, R, Q, N, E, D, S or T, without specifying further, amino acids are meant. If not otherwise indicated amino acids indicated with a single letter code in CAPITAL letters indicate the L-isoform, if however, the amino acid is indicated with a lower case letter, this amino acid is used/applied as it's D-form. Such D-forms and other non-conservative amino acid substitutions previously defined are included in a definition of unnatural amino acids.

If, due to typing errors, there are deviations from the commonly used codes, the commonly used codes apply. The amino acids present in the peptides are, preferably, amino acids which can be coded for by a nucleic acid. As is apparent from the above examples, amino acid residues may be identified by their full name, their one-letter code, and/or their three-letter code. These three ways are fully equivalent.

A “non-conservative amino acid substitution” also refers to the substitution of a member of one of these classes for a member from another class. In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those that are within ±1 are included, and in certain embodiments, those within ±0.5 are included. It is also understood in the art that the substitution of like amino acids can be made effectively on the basis of hydrophilicity, particularly where the biologically functional protein or peptide thereby created is intended for use in immunological embodiments, as disclosed herein. In certain embodiments, the greatest local average hydrophilicity of a protein, as governed by the hydrophilicity of its adjacent amino acids, correlates with its immunogenicity and antigenicity, i.e., with a biological property of the protein. The following hydrophilicity values have been assigned to these amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those that are within ±1 are included, and in certain embodiments, those within ±0.5 are included.

Other amino acid substitutions are set forth in Table 3.

TABLE 3

| Original | | Preferred |
|---|---|---|
| Residues | Substitutions | Substitutions |
| Ala | Val, Leu, Ile | Val |
| Arg | Lys, Gln, Asn | Lys |
| Asn | Gln | |
| Asp | Glu | |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn, 1,4 | Arg |
| | Diamino-butyric Acid | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

As used herein the term “charged amino acid” or “charged residue” refers to an amino acid that comprises a side chain that is negative-charged (i.e., de-protonated) or positive-charged (i.e., protonated) in aqueous solution at physiological pH. For example, negative-charged amino acids include aspartic acid, glutamic acid, cysteic acid, homocysteic acid, and homoglutamic acid, whereas positive-charged amino acids include arginine, lysine and histidine. Charged amino acids include the charged amino acids among the 20 coded amino acids, as well as atypical or non-naturally occurring or non-coded amino acids.

As used herein the term “acidic amino acid” refers to an amino acid that comprises a second acidic moiety (other than the carboxylic acid of the amino acid), including for example, a carboxylic acid or sulfonic acid group.

As used herein, the term “acylated amino acid” refers to an amino acid comprising an acyl group which is non-native to a naturally-occurring amino acid, regardless of the means by which it is produced (e.g. acylation prior to incorporating the amino acid into a peptide, or acylation after incorporation into a peptide).

As used herein the term “alkylated amino acid” refers to an amino acid comprising an alkyl group which is non-native to a naturally-occurring amino acid, regardless of the means by which it is produced. Accordingly, the acylated amino acids and alkylated amino acids of the present disclosures are non-coded amino acids.

A skilled artisan will be able to determine active variants of peptides as set forth herein using well-known techniques. In certain embodiments, one skilled in the art may identify suitable areas of the molecule that may be changed without destroying activity by targeting regions not believed to be important for activity. In other embodiments, the skilled artisan can identify residues and portions of the molecules that are conserved among similar peptides. In further embodiments, even areas that may be important for biological activity or for structure may be subject to conservative amino acid substitutions without destroying the biological activity or without adversely affecting the peptide structure. Changes in caspase activity in cells treated with a test compounds are well known to be an indicator of potential therapeutic utility. Regardless of whether caspases have been definitively implicated in the etiology or pathological consequences of a disease, a decrease in caspase activity has been associated with amelioration of the symptoms of several conditions caused by inappropriate apoptotic cell death, including diabetes, cardiovascular disease, detrimental hepatocyte apoptosis, ischemia reperfusion injury, traumatic brain injury, organ transplant, and neurodegeneration (Choadhry, J Thorac. Cardiovasc. Surg. 2007 July; 134(1):124-31; McIlwain, Cold Spring Harb. Perspect. Biol. 2013; 5:a008656). In addition, it is well known that increases in caspase activity indicates potential utility for treating diseases and disorders responsive to induction of apoptosis, including cancer, autoimmune disorders, rheumatoid arthritis, infectious diseases, inflammatory disease (Elmore, Toxicol Pathol. 2007; 35(4): 495-516). Changes in cell viability in cells treated with a test compounds are well known to be an indicator of potential therapeutic utility. A decrease in cell viability indicates potential utility for treating diseases and disorders responsive to changes in cell viability/proliferation, including for example cancer (Boyd, Drug Dev. Res., 34:91-109 (1995)). An increase in cell viability indicates potential utility for treating diseases associated with decreased cell viability, including diabetes, cardiovascular disease, ischemia reperfusion injury, traumatic brain injury, organ transplant, chemotherapy, and neurodegeneration. Additionally, an increase in cell viability indicates potential utility for improving cell viability of animal cells in culture.

Additionally, one skilled in the art can review structure-function studies identifying residues in similar peptides that are important for activity or structure. In view of such a comparison, the skilled artisan can predict the importance of amino acid residues in a peptide that correspond to amino acid residues important for activity or structure in similar peptides. One skilled in the art may opt for chemically similar amino acid substitutions for such predicted important amino acid residues.

One skilled in the art can also analyze the three-dimensional structure and amino acid sequence in relation to that structure in similar peptides. In view of such information, one skilled in the art may predict the alignment of amino acid residues of a peptide with respect to its three-dimensional structure. In certain embodiments, one skilled in the art may choose to not make radical changes to amino acid residues predicted to be on the surface of the peptide, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

The term “derivative” as used herein means a chemically modified peptide, in which one or more side chains have been covalently attached to the peptide. The term “side chain” may also be referred to as a “substituent”. A derivative comprising such side chains will thus be “derivatized” peptide or “derivatized” analog. The term may also refer to peptides containing one or more chemical moieties not normally a part of the peptide molecule such as esters and amides of free carboxy groups, acyl and alkyl derivatives of free amino groups, phospho esters and ethers of free hydroxy groups. Such modifications may be introduced into the molecule by reacting targeted amino acid residues of the peptide with an organic derivatizing agent that is capable of reacting with selected side chains or terminal residues. Preferred chemical derivatives include peptides that have been phosphorylated, C-termini amidated or N-termini acetylated. The term may also refer to peptides as used herein which may be prepared from the functional groups which occur as side chains on the residues or the N- or C-terminal groups, by means known in the art, and are included herein as long as they remain pharmaceutically acceptable, i.e., they do not destroy the activity of the peptide, do not confer toxic properties on compositions containing it and do not adversely affect the antigenic properties thereof. These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl group (for example that of seryl or threonyl residues) formed by reaction with acyl moieties.

A modified amino acid residue is an amino acid residue in which any group or bond was modified by deletion, addition, or replacement with a different group or bond, as long as the functionality of the amino acid residue is preserved or if functionality changed (for example replacement of tyrosine with substituted phenylalanine) as long as the modification did not impair the activity of the peptide containing the modified residue.

The term “substituent” or “side chain” as used herein means any suitable moiety bonded, in particular covalently bonded, to an amino acid residue, in particular to any available position on an amino acid residue. Typically, the suitable moiety is a chemical moiety.

The term “fatty acid” refers to aliphatic monocarboxylic acids having from 4 to 28 carbon atoms, it is preferably un-branched, and it may be saturated or unsaturated. In the present disclosure, fatty acids comprising 10 to 16 amino acids are preferred.

The term “fatty diacid” refers to fatty acids as defined above but with an additional carboxylic acid group in the omega position. Thus, fatty diacids are dicarboxylic acids. In the present disclosure, fatty acids comprising 14 to 20 amino acids are preferred.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm, and means that a given sequence is at least 80% identical to another length of another sequence.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence.

Exemplary computer programs which can be used to determine degrees of identity or homology between two sequences include, but are not limited to, the suite of BLAST programs, e.g., BLASTN, BLASTX, and TBLASTX, BLASTP and TBLASTN, publicly available on the Internet at the NCBI website. See also Altschul et al., 1990, J. Mol. Biol. 215:403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. (Id). In addition to calculating percent sequence identity, the BLAST algorithm also performs a statistical analysis of the similarity between two sequences (see, e.g., Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance.

A “pharmaceutical composition” refers to a composition suitable for pharmaceutical use in an animal or human A pharmaceutical composition comprises a pharmacologically and/or therapeutically effective amount of an active agent and a pharmaceutically acceptable excipient or carrier. Pharmaceutical compositions and methods for their preparation will be readily apparent to those skilled in the art. Such compositions and methods for their preparation may be found, for example, in Remington's Pharmaceutical Sciences, 19th Edition (Mack Publishing Company, 1995). The pharmaceutical compositions are generally formulated as sterile, substantially isotonic and in full compliance with all GMP regulations of the U.S. Food and Drug Administration. The term also encompasses any of the agents listed in the US Pharmacopeia for use in animals, including humans. Suitable pharmaceutical carriers and formulations are described in Remington's Pharmaceutical Sciences, 21st Ed. 2005, Mack Publishing Co, Easton.

“Pharmaceutically acceptable carrier” or “pharmaceutically acceptable excipient” refers to compositions that do not produce adverse, allergic, or other untoward reactions when administered to an animal or a human. As used herein, “pharmaceutically acceptable carrier” or “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, the excipients will include isotonic agents, for example, sugars, polyalcohols such as mannitol, sorbitol, or sodium chloride in the composition. Additional examples of pharmaceutically acceptable excipients are wetting agents or minor amounts of auxiliary substances such as wetting or emulsifying agents, preservatives or buffers, which enhance the shelf life or effectiveness of the peptide.

As used herein the term “pharmaceutically acceptable salt” refers to salts of peptides that retain the biological activity of the parent peptide, and which are not biologically or otherwise undesirable. Many of the peptides disclosed herein are capable of forming acid and/or base salts by virtue of the presence of amino and/or carboxyl groups or groups similar thereto. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines.

It may be convenient or desirable to prepare, purify, and/or handle a corresponding solvate of the peptide. The term “solvate” is used herein in the conventional sense to refer to a complex of solute (e.g., peptide, salt of peptide) and solvent. If the solvent is water, the solvate may be conveniently referred to as a hydrate, for example, a mono-hydrate, a di-hydrate, a tri-hydrate, etc. Unless otherwise specified, a reference to a particular peptide also includes solvate and hydrate forms thereof.

The “co-crystal” or “co-crystal salt” as used herein means a crystalline material composed of two or more unique solids at room temperature, each of which has distinctive physical characteristics such as structure, melting point, and heats of fusion, hygroscopicity, solubility, and stability. A co-crystal or a co-crystal salt can be produced according to a per se known co-crystallization method. The terms co-crystal (or cocrystal) or co-crystal salt also refer to a multicomponent system in which there exists a host API (active pharmaceutical ingredient) molecule or molecules, such as a peptide of Formula I, and a guest (or co-former) molecule or molecules.

As used herein, a “therapeutically effective amount” of a peptide that when provided to a subject in accordance with the disclosed and claimed methods affects biological activities such as modulating cell signaling associated with aberrant cellular proliferation and malignancy, impacting cell viability and providing neuroprotection.

The terms “treat”, “treating” and “treatment” refer refers to an approach for obtaining beneficial or desired clinical results. Further, references herein to “treatment” include references to curative, palliative and prophylactic treatment. The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

The term “improving cell survival” refers to an increase in the number of cells that survive a given condition, as compared to a control, e.g., the number of cells that would survive the same conditions in the absence of treatment. Conditions can be in vitro, in vivo, ex vivo, or in situ. Improved cell survival can be expressed as a comparative value, e.g., twice as many cells survive if cell survival is improved two-fold. Improved cell survival can result from a reduction in apoptosis, an increase in the life-span of the cell, or an improvement of cellular function and condition.

For clarity, the term “instructing” is meant to include information on a label approved by a regulatory agency, in addition to its commonly understood definition.

In an embodiment, the peptides may be administered as their nucleotide equivalents via gene therapy methods. In one embodiment, the peptide-related polynucleotide is encoded in a plasmid or vector, which may be derived from an adeno-associated virus (AAV). The AAV may be a recombinant AAV virus and may comprise a capsid serotype such as, but not limited to, of AAV1, AAV2, AAV3, AAV4, AAV5. AAV6. AAV7, AAV8, AAV9, AAV9.47, AAV9(hu14), AAV10, AAV11, AAV12, AAVrh8, AAVrh10. AAV-DJ, and AAV-DJ8. As a non-limiting example, the capsid of the recombinant AAV virus is AAV2. As a non-limiting example, the capsid of the recombinant AAV virus is AAVrh10. As a non-limiting example, the capsid of the recombinant AAV virus is AAV9(hu14). As a non-limiting example, the capsid of the recombinant AAV virus is AAV-DJ. As a non-limiting example, the capsid of the recombinant AAV virus is AAV9.47. As a non-limiting example, the capsid of the recombinant AAV virus is AAV-DJ8. An embodiment comprises the nucleotide equivalents of the peptide sequences of SEQ ID No: 1-210.

A person skilled in the art may recognize that a target cell may require a specific promoter including but not limited to a promoter that is species specific, inducible, tissue-specific, or cell cycle-specific Parr et al, Nat. Med. 3:1145-9 (1997); the contents of which are herein incorporated by reference in its entirety).

As used herein, a “vector” is any molecule or moiety which transports, transduces or otherwise acts as a carrier of a heterologous molecule such as the polynucleotides of the invention. A “viral vector” is a vector which comprises one or more polynucleotide regions encoding or comprising payload molecule of interest, e.g., a transgene, a polynucleotide encoding a polypeptide or multi-polypeptide. Viral vectors of the present invention may be produced recombinantly and may be based on adeno-associated virus (AAV) parent or reference sequence. Serotypes which may be useful in the present invention include any of those arising from AAV1, AAV2, AAV3, AAV4. AAV5, AAV6, AAV7, AAV8, AAV9, AAV9.47. AAV9(hu14), AAV10, AAV11. AAV12, AAVrh8, AAVrh1O, AAV-DJ, and AAV-DJ8.

In one embodiment, the serotype which may be useful in the present invention may be AAV-DJ8. The amino acid sequence of AAV-DJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in its entirety, may comprise two mutations: (1) R587Q where arginine (R; arg) at amino acid 587 is changed to glutamine (Q; gln) and (2) R590T where arginine (R; arg) at amino acid 590 is changed to threonine (T; thr). As another non-limiting example, may comprise three mutations: (1) K406R where lysine (K; lys) at amino acid 406 is changed to arginine (R; arg), (2) R587Q where arginine (R; arg) at amino acid 587 is changed to glutamine (Q; gln) and (3) R590T where arginine (R; arg) at amino acid 590 is changed to threonine (T; thr).

AAV vectors may also comprise self-complementary AAV vectors (scAAVs), scAAV vectors contain both DNA strands which anneal together to form double stranded DNA. By skipping second strand synthesis, scAAVs allow for rapid expression in the cell.

In one embodiment, the pharmaceutical composition comprises a recombinant adeno-associated virus (AAV) vector comprising an AAV capsid and an AAV vector genome. The AAV vector genome may comprise at least one peptide related polynucleotide described herein, such as, but not limited to, SEQ ID NO 1-210 or variants having at least 95% identity thereto. The recombinant AAV vectors in the pharmaceutical composition may have at least 70% which contain an AAV vector genome.

In one embodiment, the pharmaceutical composition comprises a recombinant adeno-associated virus (AAV) vector comprising an AAV capsid and an AAV vector genome. The AAV vector genome may comprise at least one peptide related polynucleotide described herein, such as, but not limited to, SEQ ID NO 1-210 or variants having at least 95% identity thereto, plus an additional N-terminal proline. The recombinant AAV vectors in the pharmaceutical composition may have at least 70% which contain an AAV vector genome.

In one embodiment, the viral vector comprising a peptide-related polynucleotide may be administered or delivered using the methods for the delivery of AAV virions described in European Patent Application No. EP1857552, the contents of which are herein incorporated by reference in its entirety.

In one embodiment, the viral vector comprising a peptide-related polynucleotide may be administered or delivered using the methods for delivering proteins using AAV vectors described in European Patent Application No. EP2678433, the contents of which are herein incorporated by reference in its entirety.

In one embodiment, the viral vector comprising a peptide-related polynucleotide may be administered or delivered using the methods for delivering DNA molecules using AAV vectors described in U.S. Pat. No. 5,858,351, the contents of which are herein incorporated by reference in its entirety.

In one embodiment, the viral vector comprising a peptide-related polynucleotide may be administered or delivered using the methods for delivering DNA to the bloodstream described in U.S. Pat. No. 6,211,163, the contents of which are herein incorporated by reference in its entirety.

In one embodiment, the viral vector comprising a peptide-related polynucleotide may be administered or delivered using the methods for delivering AAV virions described in U.S. Pat. No. 6,325,998, the contents of which are herein incorporated by reference in its entirety.

In one embodiment, the viral vector comprising a peptide-related polynucleotide may be administered or delivered using the methods for delivering a payload to the central nervous system described in U.S. Pat. No. 7,588,757, the contents of which are herein incorporated by reference in its entirety.

In one embodiment, the viral vector comprising a peptide-related polynucleotide may be administered or delivered using the methods for delivering a payload described in U.S. Pat. No. 8,283,151, the contents of which are herein incorporated by reference in its entirety.

In one embodiment, the viral vector comprising a peptide-related polynucleotide may be administered or delivered using the methods for delivering a payload using a glutamic acid decarboxylase (GAD) delivery vector described in International Patent Publication No. WO2001089583, the contents of which are herein incorporated by reference in its entirety.

In one embodiment, the viral vector comprising a peptide-related polynucleotide may be administered or delivered using the methods for delivering a payload to neural cells described in International Patent Publication No. WO2012057363, the contents of which are herein incorporated by reference in its entirety.

In one embodiment, the viral vector comprising a peptide-related polynucleotide may be administered or delivered using the methods for delivering a payload to cells described in U.S. Pat. No. 9,585,971, the contents of which are herein incorporated by reference in its entirety.

In one embodiment, the viral vector comprising a peptide-related polynucleotide may be administered or delivered using the methods for delivering a payload to cells described in Deverman et al. Nature Biotechnology, 34, 204-09 (2016).

In one embodiment, the viral vector comprising a peptide-related polynucleotide may be administered or delivered using the methods for the delivery of AAV virions described in U.S. Pat. No. 7,198,951 [adeno-associated virus (AAV) serotype 9 sequences, vectors containing same, and uses therefor], U.S. Pat. No. 9,217,155 [isolation of novel AAV's and uses thereof], WO2011126808 [pharmacologically induced transgene ablation system], U.S. Pat. No. 6,015,709 [transcriptional activators, and compositions and uses related thereto], U.S. Pat. No. 7,094,604 [Production of pseudotyped recombinant AAV virions], WO2016126993 [anti-tau constructs], U.S. Pat. No. 7,094,604 [recombinant AAV capsid protein], U.S. Pat. No. 8,292,769 [Avian adenoasssocited viru (aaav) and uses thereof], U.S. Pat. No. 9,102,949 [CNS targeting aav vectors and methods of use thereof], US20160120960 [adeno-associated virus mediated gene transfer to the central nervous system], WO2016073693 [AADC polynucleotides for the treatment of parkinson's disease], WO2015168666 [AAV VECTORS FOR RETINAL AND CNS GENE Therapy], US20090117156 [Gene Therapy for Niemann-Pick Disease type A] or WO2005120581 [gene therapy for neurometabolic disorders].

The pharmaceutical compositions of viral vectors described herein may be characterized by one or more of bioavailability, therapeutic window and/or volume of distribution.

In some embodiments, peptide-related nucleotides and/or peptide-related nucleotide compositions of the present invention may be combined with, coated onto or embedded in a device. Devices may include, but are not limited to stents, pumps, and/or other implantable therapeutic device. Additionally, peptide-related nucleotides and/or peptide-related nucleotide compositions may be delivered to a subject while the subject is using a compression device such as, but not limited to, a compression device to reduce the chances of deep vein thrombosis (DVT) in a subject. The present invention provides for devices which may incorporate viral vectors that encode one or more peptide-related polynucleotide payload molecules. These devices contain in a stable formulation the viral vectors which may be immediately delivered to a subject in need thereof, such as a human patient.

Devices for administration may be employed to deliver the viral vectors comprising an peptide-related nucleotides of the present invention according to single, multi- or split-dosing regimens taught herein.

As used herein and in the appended claims, the singular forms “a,” “or,” and “the” include plural referents unless the context clearly dictates otherwise. It is understood that aspects and variations of the disclosure described herein include “consisting” and/or “consisting essentially of” aspects and variation.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age which suffer from the pathology. Preferably, this term encompasses individuals who are at risk to develop the pathology.

The pharmaceutical compositions are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous injection, intraperitoneal injection, intramuscular injection, intrasternal injection, intravenous injection, intraarterial injection, intrathecal injection, intraventricular injection, intraurethral injection, intracranial injection, intrasynovial injection or infusions; or kidney dialytic infusion techniques.

In various embodiments, the peptide is admixed with a pharmaceutically acceptable excipients to form a pharmaceutical composition that can be systemically administered to the subject orally or via intravenous injection, intramuscular injection, subcutaneous injection, intraperitoneal injection, transdermal injection, intra-arterial injection, intrasternal injection, intrathecal injection, intraventricular injection, intraurethral injection, intracranial injection, intrasynovial injection or via infusions. The pharmaceutical composition preferably contains at least one component that is not found in nature.

Formulations of a pharmaceutical composition suitable for parenteral administration typically generally comprise the active ingredient combined with a pharmaceutically acceptable excipient, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain carriers such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

The present disclosure includes compositions and methods for transdermal or topical delivery, to act locally at the point of application, or to act systemically once entering the body's blood circulation. In these systems, delivery may be achieved by techniques such as direct topical application of a substance or drug in the form of an ointment or the like, or by adhesion of a patch with a reservoir or the like that holds the drug (or other substance) and releases it to the skin in a time-controlled fashion. For topical administration, the compositions can be in the form of emulsions, lotions, gels, creams, jellies, solutions, suspensions, ointments, and transdermal patches. Some topical delivery compositions may contain polyenylphosphatidylcholine (herein abbreviated “PPC”). In some cases, PPC can be used to enhance epidermal penetration. The term “polyenylphosphatidylcholine,” as used herein, means any phosphatidylcholine bearing two fatty acid moieties, wherein at least one of the two fatty acids is an unsaturated fatty acid with at least two double bonds in its structure, such as linoleic acid. Such topical formulations may comprise one or more emulsifiers, one or more surfactants, one or more polyglycols, one or more lecithins, one or more fatty acid esters, or one or more transdermal penetration enhancers. Preparations can include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, organic esters such as ethyl oleate, or fixed oils including synthetic mono or di-glycerides. Aqueous solvents include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like.

For example, in one aspect, sterile injectable solutions can be prepared by incorporating a peptide in the required amount in an appropriate solvent with one or a combination of ingredients enumerated above, as required, followed by filtered sterilization. Generally, dispersions are prepared by incorporating the active peptide into a sterile vehicle that contains a basic dispersion medium and the required other ingredients from those enumerated above. In the case of sterile powders for the preparation of sterile injectable solutions, methods of preparation such as vacuum drying and freeze-drying yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin. In various embodiments, the injectable compositions will be administered using commercially available disposable injectable devices.

The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind known in the art. Injectable formulations are in accordance with the disclosure. The requirements for effective pharmaceutical excipients for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Additionally, the peptides of the present disclosures can be made into suppositories for rectal administration by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

It will be appreciated by one of skill in the art that, in addition to the above-described pharmaceutical compositions, the peptides of the disclosure can be formulated as inclusion complexes, such as cyclodextrin inclusion complexes, or liposomes.

The peptide can be administered intranasally or by inhalation, typically in the form of a dry powder (either alone, as a mixture, or as a mixed component particle, for example, mixed with a suitable pharmaceutically acceptable carrier) from a dry powder inhaler, as an aerosol spray from a pressurized container, pump, spray, atomiser (preferably an atomiser using electrohydrodynamics to produce a fine mist), or nebulizer, with or without the use of a suitable propellant, or as nasal drops. The pressurized container, pump, spray, atomizer, or nebulizer generally contains a solution or suspension of a peptide comprising, for example, a suitable agent for dispersing, solubilizing, or extending release of the active, a propellant(s) as solvent. Prior to use in a dry powder or suspension formulation, the drug product is generally micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying. Capsules, blisters and cartridges for use in an inhaler or insufflator may be formulated to contain a powder mix of the peptide, a suitable powder base and a performance modifier. Suitable flavors, such as menthol and levomenthol, or sweeteners, such as saccharin or saccharin sodium, may be added to those formulations intended for inhaled/intranasal administration. Formulations for inhaled/intranasal administration may be formulated to be immediate and/or modified release. Modified release formulations include delayed-, sustained-, pulsed-, controlled-, targeted and programmed release. In the case of dry powder inhalers and aerosols, the dosage unit is determined by means of a valve which delivers a metered amount. Units are typically arranged to administer a metered dose or “puff” of a peptide. The overall daily dose will typically be administered in a single dose or, more usually, as divided doses throughout the day.

According to one aspect, the peptides are for use in medicine, particularly human medicine. The peptides are effective to modulate cell signaling associated with aberrant cellular proliferation and malignancy. Additionally, the disclosure provides peptides effective in impacting cell viability and cytoprotection.

In some aspects, methods are provided herein for treating a condition for which apoptotic cell death, inflammation, autoimmunity, angiogenesis, and/or metastasis is an etiological determinant.

In another aspect, there is provided a peptide, for use in in the prevention and/or treatment of bone- or cartilages disorders/diseases, cancer, autoimmune diseases, fibrotic diseases, inflammatory diseases, obesity, type I and type II diabetes, neurodegenerative diseases, bone fractures, skeletal chondrodysplasias, infectious diseases, lung diseases, infertility, muscular disorders, aging, skin diseases, and metabolic diseases.

In some aspects, the peptides are administered to treat a condition associated with cellular stress responses, such as but not limited to, the induction of heat shock proteins and/or metabolic and oxidative stress. The cellular stress response can be responsive to any stressor, including, e.g., thermal, immunological, cytokine, oxidative, metabolic, anoxic, endoplasmic, reticulum, protein unfolding, nutritional, chemical, mechanical, osmotic and glycemic stresses.

In some aspects, peptides are administered according to a method provided herein to treat an inflammatory condition, such as but not limited to, diabetes, cardiovascular disease, kidney disease, retinopathy, obesity, metabolic disease, neurodegenerative disease, gastrointestinal disease, autoimmune disease, rheumatological disease or infectious disease.

Without being bound by a specific theory, free fatty acids (FFA) in cell culture media after treatment of adipocytes with the peptides indicates a modulation of pathways involved in cellular regulation of lipid or fatty acid levels. Decreases in fatty acid levels in the media may result from a number of processes, including but not limited to inhibition of signaling pathways, reduction in cellular lipogenesis, reduction in lipolysis, or increase in fatty acid oxidation. Peptides that have an effect on the net concentration of free fatty acids have potential utility for treatment of metabolic disorders.

The peptides are useful in the treatment of conditions associated with an unbalanced metabolic state manifested by abnormal blood levels of glucose, reactive oxygen species (ROS) and/or free fatty acids (FFA). A favorable metabolic status is defined as a balanced energy homeostasis, characterized by blood levels of glucose, ROS and FFA that are equivalent to those of healthy subjects (within the range of average levels for the healthy population). Accordingly, an unfavorable metabolic status as used herein refers to blood levels of glucose, ROS and/or FFA that are abnormal, i.e. significantly altered compared to their respective levels in healthy control subjects (e.g. as evaluated by a physician or skilled artisan). The term unfavorable metabolic status refers in some embodiments to blood levels of glucose, ROS and/or FFA that are significantly enhanced compared to their respective levels in healthy control subjects (e.g. as evaluated by a physician or skilled artisan). An unfavorable metabolic status may result from abnormal metabolism which may involve glucose (carbohydrate) and/or fatty acid oxidation pathways. When aberrations in fatty acid oxidation pathways are involved, the unfavorable metabolic status is typically manifested by ROS blood levels that are significantly enhanced compared to healthy control subjects and/or by abnormal FFA blood levels. These aberrations may also be manifested by elevated blood levels of oxidized low density lipoproteins (LDL). When aberrations in glucose metabolism are involved, glucose blood levels are typically significantly enhanced compared to healthy control subjects. As used herein, a patient with significantly enhanced blood glucose levels that do not exceed the threshold for unbalanced glycemic control will be defined as having an unfavorable metabolic status if said enhancement is accompanied by abnormal blood ROS and/or FFA values, as described herein. An unbalanced metabolic state may also be evaluated by said physician or skilled artisan by considering the energy intake and various energy consumption and utilization parameters, as known in the art. For example, without limitation, parameters at the cellular level such as cellular (e.g. platelet) ATP production and cellular oxidation, and parameters at the whole body level such as respiratory quotient (RQ) may be evaluated to determine the metabolic status of the subject. For example, by comparing the relative ratio of such parameters between healthy and sick patients the skilled artisan may evaluate the metabolic status of the subject compared to healthy controls. An unfavorable metabolic status may be found in patients afflicted with chronic metabolic and/or inflammatory disorders that are not adequately treated or balanced by a suitable therapeutic regimen.

The term “metabolic disease” or “metabolic disorder” refers to a group of identified disorders in which errors of metabolism, imbalances in metabolism, or sub-optimal metabolism occur, which may involve glucose (carbohydrate), fatty acid and/or protein oxidation pathways. Accordingly, when unbalanced, these disorders are typically manifested by an unfavorable metabolic status characterized by abnormal blood levels of glucose, ROS and/or FFA compared to their respective levels in healthy control subjects, as described herein. Such disorders include without limitation diabetes and disorders associated with nutritional or endocrine imbalance.

An unfavorable metabolic status may also occur as a result of chronic inflammatory disorders, in which a non-resolving, unbalanced inflammatory process is accompanied by secondary metabolic complications manifested by abnormal blood levels of glucose, ROS and/or FFA compared to their respective levels in healthy control subjects. Non-limitative examples of such disorders are sepsis and autoimmune diseases.

Syndrome X (or metabolic syndrome) denotes a set of signs and symptoms associated with the accumulation of fat in the abdomen. This form of fat distribution is common in middle-aged men and is often visible as a pot belly or paunch. Syndrome X is characterized by a number of disorders including gout, impaired glucose metabolism (increasing susceptibility to diabetes), raised blood pressure, and elevated blood cholesterol levels. People with Syndrome X have a high risk of heart disease. Syndrome X is defined as a constellation of metabolic abnormalities in serum or plasma insulin/glucose level ratios, lipids, uric acid levels, vascular physiology, and coagulation factor imbalances by the American Association of Clinical Endocrinologists. The term “syndrome X” as used herein thus refers to a condition characterized by positive diagnosis of at least two of the following: Non-insulin-dependent diabetes, blood pressure above a level considered normal, insulin level above a level considered normal, dyslipidemia, and obesity.
A peptide may be useful in the following metabolic diseases
(a) prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C;
(b) delaying or preventing diabetic disease progression, such as progression in type 2 diabetes, delaying the progression of impaired glucose tolerance (IGT) to insulin requiring type 2 diabetes, delaying or preventing insulin resistance, and/or delaying the progression of non-insulin requiring type 2 diabetes to insulin requiring type 2 diabetes;
(c) improving p-cell function, such as decreasing p-cell apoptosis, increasing p-cell function and/or p-cell mass, and/or for restoring glucose sensitivity to p-cells;
(d) prevention and/or treatment of cognitive disorders and/or neurodegenerative disorders, such as Alzheimer's disease, Parkinson's disease, and/or multiple sclerosis;
(e) prevention and/or treatment of eating disorders, such as obesity, e.g. by decreasing food intake, reducing body weight, suppressing appetite, inducing satiety; treating or preventing binge eating disorder, bulimia nervosa, and/or obesity induced by administration of an antipsychotic or a steroid; reduction of gastric motility; delaying gastric emptying; increasing physical mobility; and/or prevention and/or treatment of comorbidities to obesity, such as osteoarthritis and/or urine incontinence;
(f) prevention and/or treatment of diabetic complications, such as angiopathy; neuropathy, including peripheral neuropathy; nephropathy; and/or retinopathy;
(g) improving lipid parameters, such as prevention and/or treatment of dyslipidemia, lowering total serum lipids; increasing HDL; lowering small, dense LDL; lowering VLDL;
lowering triglycerides; lowering cholesterol; lowering plasma levels of lipoprotein a (Lp(a)) in a human; inhibiting generation of apolipoprotein a (apo(a)) in vitro and/or in vivo;
(h) prevention and/or treatment of cardiovascular diseases, such as syndrome X, atherosclerosis, myocardial infarction, coronary heart disease, reperfusion injury, stroke, hypoxia, cerebral ischemia, an early cardiac or early cardiovascular disease, left ventricular hypertrophy, coronary artery disease, hypertension, essential hypertension, acute hypertensive emergency, cardiomyopathy, heart insufficiency, exercise intolerance, acute and/or chronic heart failure, arrhythmia, cardiac dysrhythmia, syncopy, angina pectoris, cardiac bypass and/or stent reocclusion, intermittent claudication (atherosclerosis obliterans), diastolic dysfunction, and/or systolic dysfunction; and/or reduction of blood pressure, such as reduction of systolic blood pressure;
(i) prevention and/or treatment of gastrointestinal diseases, such as inflammatory bowel disease, short bowel syndrome, or Crohn's disease or colitis; dyspepsia, and/or gastric ulcers; and/or inflammation, such as psoriasis, psoriatic arthritis, rheumatoid arthritis, and/or systemic lupus erythematosus;
(j) prevention and/or treatment of critical illness, such as treatment of a critically ill patient, a critical illness poly-nephropathy (CIPNP) patient, and/or a potential CIPNP patient; prevention of development of critical illness or CIPNP; prevention, treatment and/or cure of systemic inflammatory response syndrome (SIRS) in a patient; prevention or reduction of the likelihood of a patient suffering from bacteremia, septicemia, and/or septic shock during hospitalization; and/or stabilizing blood glucose, insulin balance and optionally metabolism in intensive care unit patients with acute illness;
(k) prevention and/or treatment of polycystic ovary syndrome (PCOS);
(l) prevention and/or treatment of cerebral disease, such as cerebral ischemia, cerebral hemorrhage, and/or traumatic brain injury;
(m) prevention and/or treatment of sleep apnea;
(n) prevention and/or treatment of abuse, such as alcohol abuse and/or drug abuse;
(o) prevention or treatment of fatty liver conditions, including but not limited to Fatty Liver Disease (FLD), nonalcoholic fatty liver disease (NAFLD), and nonalcoholic steatohepatitis (NASH); and/or
(p) treatment of intracellular production of reactive oxygen species (ROS).

In further aspects, methods are provided herein for treating diabetes and/or diabetes related complications by administering an effective amount, of the peptides to a patient in need of treatment. Advantageously, the peptides used for treating diabetes and/or related complications according to methods provided herein have anti-apoptotic activity against and/or stimulate proliferation of pancreatic p cells, such that administering the peptides increases the number of insulin producing § cells and the level of insulin produced by the patient.

The present disclosure also includes methods of treating cancer comprising administering an effective amount of a peptide or a variant thereof to a subject in need of treatment. The peptides provided herein exert a variety of anticancer effects and can be used to treat a wide range of cancers and other proliferative disorders. Peptides provided herein can have a variety of anticancer activities, such as but not limited to, inducing apoptosis in cancerous cells, inhibiting tumor angiogenesis, inhibiting tumor metastasis, modulating the cell cycle, inhibiting cancer cell proliferation, promoting cancer cell differentiation, inhibiting production of and/or protecting against reactive oxygen species, and enhancing stress resistance. A “cancer” refers generally to a disease characterized by uncontrolled, abnormal cell growth and proliferation. A “tumor” or “neoplasm” is an abnormal mass of tissue that results from excessive, un controlled, and progressive cell division. Methods described herein are useful for treating cancers and proliferative disorders of any type, including but not limited to, carcinomas, sarcomas, soft tissue sarcomas, lymphomas, hematological cancers, leukemias, germ cell tumors, and cancers without solid tumors (e.g., hematopoietic cancers). In various aspects, the peptides can be used to treat cancers and/or tumors originating from and/or effecting any tissue, including but not limited to, lung, breast, epithelium, large bowel, rectum, testicle, bladder, thyroid, gallbladder, bile duct, biliary tract, prostate, colon, stomach, esophagus, pancreas, liver, kidney, uterus, cervix, ovary, and brain tissues. Non-limiting examples of specific cancers treatable with the peptides include, but are not limited to, acute lymphoblastic leukemia, acute myeloid leukemia, chronic lymphocytic leukemia, chronic myelogenous leukemia, adrenocortical carcinoma, AIDS-related lymphoma, anal cancer, astrocytoma, cerebral basal cell carcinoma, bile duct cancer, extrahepatic bladder cancer, bladder cancer, bone cancer, osteosarcoma/malignant fibrous histiocytoma, brain stem glioma, brain tumor, brain stem glioma, cerebral astrocytoma/malignant glioma, ependymoma, medulloblastoma, supratentorial primitive neuroectodermal tumor, visual pathway and hypothalamic glioma, breast cancer, male bronchial adenomas/carcinoids, Burkitt's lymphoma, carcinoid tumor, gastrointestinal carcinoma of unknown primary central nervous system lymphoma, cervical cancer, chronic lymphocytic leukemia, chronic myelogenous leukemia, chronic myeloproliferative disorders, colon cancer, colorectal cancer, cutaneous t-cell lymphoma, mycosis fungoides and sezary syndrome, endometrial cancer, ependymoma, esophageal cancer, Ewing's family tumors, germ cell tumors, extrahepatic bile duct cancer, eye cancer, intraocular melanoma, retinoblastoma, gallbladder cancer, gastric (stomach) cancer, gastrointestinal carcinoid tumors, ovarian gestational, trophoblastic tumors, glioma, hypothalamic skin cancer (melanoma), skin cancer (non-melanoma), skin carcinoma, small cell lung cancer, small intestine cancer, soft tissue sarcoma, squamous cell carcinoma, squamous neck cancer with occult primary, metastatic stomach (gastric) cancer, stomach (gastric) cancer, t-cell lymphoma, testicular cancer, thymoma, thymic carcinoma, thyroid cancer, transitional cell cancer of the renal pelvis, ureter trophoblastic tumors, transitional cell cancer, urethral cancer, uterine cancer, uterine sarcoma, vaginal cancer, hypothalamic glioma, vulvar cancer, Waldenstrom's macroglobulinemia, Wilms' tumor, hairy cell leukemia, head and neck cancer, hepatocellular (liver) cancer, Hodgkin's lymphoma, hypopharyngeal cancer, islet cell carcinoma (endocrine pancreas), Kaposi's sarcoma, kidney (renal cell) cancer, kidney cancer, laryngeal cancer, hairy cell lip and oral cavity cancer, liver cancer, lung cancer, non-small cell lung cancer, small cell lymphoma, Burkitt's lymphoma, cutaneous t-cell, Hodgkin's lymphoma, non-Hodgkin's lymphoma, Waldenstrom's malignant fibrous histiocytoma of bone/osteosarcoma medulloblastoma, intraocular (eye) merkel cell carcinoma, mesothelioma, malignant mesothelioma, metastatic squamous neck cancer with occult primary multiple endocrine neoplasia syndrome, multiple myeloma/plasma cell neoplasm, mycosis fungoides myelodysplastic syndromes, myelodysplastic/myeloproliferative diseases, myelogenous leukemia, multiple myeloproliferative disorders, chronic nasal cavity and paranasal sinus cancer, nasopharyngeal cancer, pleoropulmonary blastoma, osteosarcoma/malignant fibrous histiocytoma of bone, pheochromocytoma, pineoblastoma, and supratentorial primitive neuroectodermal tumors. In some preferred aspects, the cancer is breast cancer. In some preferred aspects, the cancer is prostate cancer.

In some aspects, administering a peptide according to a method provided herein enhances efficacy of an established cancer therapy. In further aspects, administering a peptide according to a method provided herein enhances the anticancer activity of another cancer therapy, such as radiation or chemotherapy. In some aspects, methods are provided herein for inducing cell death in cancer cells and/or tumor cells, the methods comprising administering a peptide described herein in an amount sufficient to induce cancer cell death and/or tumor cell death.

In some embodiments, the peptides have one or more cell protective or cytoprotective activities. For example, in some aspects, the peptides are capable of preventing cell damage, improving cell survival, and/or enhancing resistance to environmental stress, such as but not limited to, heat shock, serum withdrawal, chemotherapy, and/or radiation.

In some aspects, administering a peptide according to a method provided herein decreases adverse effects of an established cancer therapy.

The methods disclosed herein include neuroprotection, treating conditions associated with the integrity and function of, or treat damage to, any of the tissues or cells of the CNS, and particularly the neurons, glial cells, or endothelial cells, from a condition, disease or event that would otherwise result in damage to such tissues or cells or to the integrity of the blood-brain barrier. Such neuroprotection serves to prevent, reduce or treat the damage that would otherwise occur to such tissues or cells caused by such condition, disease or event. Such methods include treatment of traumatic spinal cord injury, traumatic brain injury, multiple sclerosis, peripheral nerve injury, and ischemic or hemorrhagic stroke.

In particular, the peptides may be effective in the protection of white blood cells from suppression, protecting germ cells from cell death induced by a chemotherapeutic agent and inhibiting a reduction or decrease in fertility induced by a chemotherapeutic agent.

For example, in some aspects, administering a peptide according to a method provided herein protects non-cancerous cells against the adverse effects of a non-specific cancer therapy, such as radiation or chemotherapy.

In some embodiments, the peptides provided herein have neuroprotective activity against neurotoxicity in the peripheral nervous system, such as but not limited to, neurotoxicity associated with chemotherapeutic agents, radiation therapy, anti-infective agents, and/or other therapeutics. For example, the peptides provided herein may exert neuroprotective activity against peripheral neurotoxicity associated with Vinca alkaloids, platinum compounds, suramin, taxanes, and/or other chemotherapeutic agents.

In some embodiments, the peptides exhibit cell survival promoting (e.g., anti-apoptotic) activity against disease-associated cells and/or stimuli, such as but not flouted to, cells of subjects suffering from diabetes, kidney disease, and/or cancer. For example, in some aspects, the peptides have anti-apoptotic activity against pancreatic P-cells of diabetic subjects and/or tumor cells.

Advantageously, administering a peptide according to methods provided herein provides a protective effect against neurodegenerative effects, including for example, cell death induced by the SOD1 mutant in amyotrophic lateral sclerosis subjects, mutant APP, PS-1, PS-22, or amyloid-beta (Aβ) peptides in Alzheimer's disease subjects, and/or polyglutamine repeat mutations in Huntington's disease subjects.

In some embodiments, the peptides provided herein have cell growth-stimulating activity against disease-associated cells, such as but not limited to, pancreatic P-cells of diabetic subjects.

In some embodiments, the peptides provided herein have differentiation-stimulating activity against disease-associated cells. For example, in some aspects, the peptides stimulate insulin-induced differentiation of adipocytes front diabetic patients.

In some embodiments, the peptides have anticancer activity. For example, in some aspects, the peptides have pro-apoptotic activity against cancer cells, such as but not limited to, prostate cancer cells and/or breast cancer cells. In further aspects, the peptides have anti-proliferative activity against cancer cells, such as but not limited to, prostate cancer cells and/or breast cancer cells.

Further preferred medical uses include treatment or prevention of degenerative disorders, particularly neurodegenerative disorders such as Alzheimer's disease, Parkinson's disease, Huntington's disease, ataxia, e.g. spinocerebellar ataxia, Kennedy disease, myotonic dystrophy, Lewy body dementia, multi-systemic atrophy, amyotrophic lateral sclerosis, primary lateral sclerosis, spinal muscular atrophy, prion-associated diseases, e.g. Creutzfeldt-Jacob disease, multiple sclerosis, telangiectasia, Batten disease, corticobasal degeneration, corticobasal degeneration, subacute combined degeneration of spinal cord, Tabes dorsalis, Tay-Sachs disease, toxic encephalopathy, infantile Refsum disease, Refsum disease, neuroacanthocytosis, Niemann-Pick disease, Lyme disease, Machado-Joseph disease, Sandhoff disease, Shy-Drager syndrome, wobbly hedgehog syndrome, proteopathy, cerebral p-amyloid angiopathy, retinal ganglion cell degeneration in glaucoma, synucleinopathies, tauopathies, frontotemporal lobar degeneration (FTLD), dementia, cadasil syndrome, hereditary cerebral hemorrhage with amyloidosis, Alexander disease, seipinopathies, familial amyloidotic neuropathy, senile systemic amyloidosis, serpinopathies, AL (light chain) amyloidosis (primary systemic amyloidosis), AH (heavy chain) amyloidosis, AA (secondary) amyloidosis, aortic medial amyloidosis, ApoAI amyloidosis, ApoAII amyloidosis, ApoAIV amyloidosis, familial amyloidosis of the Finnish type (FAF), Lysozyme amyloidosis, Fibrinogen amyloidosis, Dialysis amyloidosis, Inclusion body myositis/myopathy, Cataracts, Retinitis pigmentosa with rhodopsin mutations, medullary thyroid carcinoma, cardiac atrial amyloidosis, pituitary prolactinoma, Hereditary lattice corneal dystrophy, Cutaneous lichen amyloidosis, Mallory bodies, corneal lactoferrin amyloidosis, pulmonary alveolar proteinosis, odontogenic (Pindborg) tumor amyloid, cystic fibrosis, sickle cell disease or critical illness myopathy (CIM). Without being limited by a particular theory, it is believed that the peptides provided herein have one or more activities capable of repairing and/or preventing neurodegenerative damage of neural cells and/or other cell types. “Neurodegenerative diseases” treatable according to methods provided herein are progressive diseases resulting in the degeneration and/or loss of neurons, for example due to neuronal cell death (apoptosis). Examples of neurodegenerative diseases include, but are not limited to, cerebral degenerative diseases (e.g., Alzheimer's disease (AD), Parkinson's disease, progressive supranuclear palsy, and Huntington's disease (HD)), and spinal degenerative disease/motor neuron degenerative diseases (e.g., amyotrophic lateral sclerosis (ALS). (SMA: Werdnig-Hoffmann disease or Kugelberg-Welander syndrome), spinocerebellar ataxia, bulbospinal muscular atrophy (BSMA; Kennedy-Alter-Sung syndrome)). A “motor neuron degenerative disease” is a neurodegenerative disease characterized by a progressive, retrograde disorder of upper and lower motor neurons that control motion in the body. In further aspects, the peptides and compositions thereof are also effective in ameliorating conditions resulting from motor neuron degenerative disease, such as muscular atrophy, muscular weakness, bulbar palsy (muscular atrophy or weakness in the face, pharynx, and tongue, and aphasia or dysphagia caused thereby), muscular fasciculation, and respiratory disorder.

Further uses include the prevention and treatment of diseases or conditions associated with mitochondrial dysfunction. Mitochondria, central to metabolic processes, are involved with energy production, programmed cell death, and reactive oxygen species (ROS) generation. Traditionally, mitochondria have been considered as “end-function” organelles, receiving and processing vast amounts of cellular signals to regulate energy production and cell death. The peptides and pharmaceutical formulations thereof can be used to treat various age-related disease with much metabolic implications. Also they have an impact on has also been tested in various ways in vitro and in vivo to affect mitochondrial respiration, glucose transport, glucose utilization, glycolysis, insulin regulation and cellular proliferation/survival. Mitochondrial dysfunction is associated with but not limited to metabolic disorders, neurodegenerative diseases, chronic inflammatory diseases, and diseases of aging. Some mitochondrial diseases are due to mutations or deletions in the mitochondrial genome. Mitochondria divide and proliferate with a faster turnover rate than their host cells, and their replication is under control of the nuclear genome. If a threshold proportion of mitochondria in a cell is defective, and if a threshold proportion of such cells within a tissue have defective mitochondria, symptoms of tissue or organ dysfunction can result. Practically any tissue can be affected, and a large variety of symptoms may be present, depending on the extent to which different tissues are involved. In addition to congenital disorders involving inherited defective mitochondria, acquired mitochondrial dysfunction contributes to diseases, particularly neurodegenerative disorders associated with aging like Parkinson's, Alzheimer's, and Huntington's Diseases. The incidence of somatic mutations in mitochondrial DNA rises exponentially with age; diminished respiratory chain activity is found universally in aging people. Mitochondrial dysfunction is also implicated in excitotoxic neuronal injury, such as that associated with seizures or ischemia. Other disorders associated with mitochondrial dysfunction include chronic inflammatory disorders and metabolic disorders.

Peptides that are cytoprotective have potential utility to extend the viability of cells in culture. The peptides are useful for manufacture of biological products, including proteins, antibodies and the like. The present disclosure relates generally to peptides and processes for modulating one or more properties of a cell culture, including mammalian cell cultures such as CHO cell cultures, or E. coli cell cultures. In one embodiment, there is provided a method of increasing specific productivity in a mammalian cell culture expressing a recombinant protein comprising establishing a mammalian cell culture in a culture medium; increasing cell growth viability by contacting the cell culture with a culture medium comprising a peptide; and maintaining the cell culture by contacting the culture with a culture medium comprising a peptide.

According to another embodiment, the peptides are coadministered or co-formulated with other known chemotherapeutic agents and/or anti-inflammatory agents.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present disclosure.

It is to be noted that dosage values may vary with the type and severity of the condition to be ameliorated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the subject, the severity of the condition, the route of administration, and the particular peptide employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-subject dose-escalation as determined by the skilled artisan. Determining appropriate dosages and regimens are well-known in the relevant art and would be understood to be encompassed by the skilled artisan once provided the teachings disclosed herein.

The dose of the peptide of the present disclosure also will be determined by the existence, nature and extent of any adverse side effects that might accompany the administration of a particular peptide of the present disclosure. Typically, the attending physician will decide the dosage of the peptide of the present disclosure with which to treat each individual patient, taking into consideration a variety of factors, such as age, body weight, general health, diet, sex, peptide of the present disclosure to be administered, route of administration, and the severity of the condition being treated. By way of example and not intending to be limiting, the dose of the peptide of the present disclosure can be about 0.0001 to about 100 mg/kg body weight of the subject being treated/day, from about 0.001 to about 10 mg/kg body weight/day, or about 0.01 mg to about 1 mg/kg body weight/day. The peptide can be administered in one or more doses, such as from 1 to 3 doses.

In some embodiments, the pharmaceutical composition comprises any of the analogs disclosed herein at a purity level suitable for administration to a patient. In some embodiments, the analog has a purity level of at least about 90%, preferably above about 95%, more preferably above about 99%, and a pharmaceutically acceptable diluent, carrier or excipient.

The pharmaceutical compositions may be formulated to achieve a physiologically compatible pH. In some embodiments, the pH of the pharmaceutical composition may be at least 5, or at least 6, or at least 7, depending on the formulation and route of administration.

In various embodiments, single or multiple administrations of the pharmaceutical compositions are administered depending on the dosage and frequency as required and tolerated by the subject. In any event, the composition should provide a sufficient quantity of at least one of the peptide disclosed herein to effectively treat the subject. The dosage can be administered once but may be applied periodically until either a therapeutic result is achieved or until side effects warrant discontinuation of therapy.

The dosing frequency of the administration of the peptide pharmaceutical composition depends on the nature of the therapy and the particular disease being treated. The administration may be once, twice, three times or four times daily, for the peptide. Treatment of a subject with a therapeutically effective amount of a peptide, can include a single treatment or, preferably, can include a series of treatments. In a preferred example, a subject is treated with peptide daily, one time per week or biweekly.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

### EMBODIMENTS

The embodiments listed below are presented in numbered form for convenience and for ease and clarity of reference in referring back to multiple embodiments. The invention provides:

1. A peptide comprising any of the following amino acid sequences:

SEQ ID NO: 1
MAAAPSGCPDPTS;
SEQ ID NO: 2
MAASVERGFIFLV;
SEQ ID NO: 3
MAGGFMLMIVVMKLMAPKMEETPA;
SEQ ID NO: 4
MAHTFPLNKTSR;
SEQ ID NO: 5
MAHYSQIPSRPHGWPPSD;
SEQ ID NO: 6
MAIAVVYPKTTIIPPK;
SEQ ID NO: 7
MAIMIINDEYWLVVLVMVHCPESMLLK;
SEQ ID NO: 8
MAPPLPPLEPTVKLT;
SEQ ID NO: 9
MAQTMPMYPNGSFFPE;
SEQ ID NO: 10
MATKKNFMEKGTRAGDMGSKPHS;
SEQ ID NO: 11
MAVVDGWVNTWVVPKSASPWKN;
SEQ ID NO: 12
MAYEGCCYSCKQEDNADVSGFWVEKWSVM;
SEQ ID NO: 13
MCSCVYVYSYCKYMVCSHDKP;
SEQ ID NO: 14
MDGCALLAVL;
SEQ ID NO: 15
MDMVPQGKDEKL;
SEQ ID NO: 16
MDSWVVGVNEWG;
SEQ ID NO: 17
MDWSNWVWGVQLYVWDFLGSGCWAWTLS;
SEQ ID NO: 18
MEAMGLAWNQLWGVRFLPFLS;
SEQ ID NO: 19
METDLDYSGLNSDHVGL;
SEQ ID NO: 20
MFDQENVVTSTESSPS;
SEQ ID NO: 21
MFEELINVWVWVYMSQWEFYDGPCNEQCY;
SEQ ID NO: 22
MFGSEFMYHSENSMMDHVTNNATGMNIMEK;
SEQ ID NO: 23
MFHLRYHNHRYPHRRQSI;
SEQ ID NO: 24
MFKLSFYFYVV;
SEQ ID NO: 25
MFLVGWWGMV;
SEQ ID NO: 26
MGACQGGSDESN;
SEQ ID NO: 27
MGCDDNVVDVVVT;
SEQ ID NO: 28
MGCSGSSVSQCYRVHTPQTKMPNAW;
SEQ ID NO: 29
MGDCAVCDA;
SEQ ID NO: 30
MGEGLEENPTNPITKPTLN;
SEQ ID NO: 31
MGEVGLLWLGRI;
SEQ ID NO: 32
MGGEWGMGVWTWGCFLVWM;
SEQ ID NO: 33
MGGGVLLGLQ;
SEQ ID NO: 34
MGGLGWGWPGGWVWGGGFMVEERWWEL;
SEQ ID NO: 35
MGGRWGLGWWRAG;
SEQ ID NO: 36
MGLAAVCVCWVGWAGVVLM;
SEQ ID NO: 37
MGLLEWLLCWHLLGRIINWWA;
SEQ ID NO: 38
MGLWLVCWVL;
SEQ ID NO: 39
MGNIIRAWCGEGCL;
SEQ ID NO: 40
MGSPPPAGSKKVVL;
SEQ ID NO: 41
MGSSRINPDPSPS;
SEQ ID NO: 42
MGSSRLAVLCPPLHGQVNFTG;
SEQ ID NO: 43
MGVGMEWGLLLRRGR;
SEQ ID NO: 44
MGVKFFTLST;
SEQ ID NO: 45
MHGELPWVVN;
SEQ ID NO: 46
MHHMKHPIIC;
SEQ ID NO: 47
MHKHYYNKHPHHYNLP;
SEQ ID NO: 48
MHKRLSPIYYSHRYLPDKRL;
SEQ ID NO: 49
MHLVFSSGGYARDSIA;
SEQ ID NO: 50
MHQSCKPEMKTFFQGQI;
SEQ ID NO: 51
MIEMGASFCHV;
SEQ ID NO: 52
MITVPAHKGMAMA;
SEQ ID NO: 53
MIYVSSDVGDWWKGGWGVWWVVHG;
SEQ ID NO: 54
MKFEIWLGWC;
SEQ ID NO: 55
MKHRQVLWVLSCGS;
SEQ ID NO: 56
MKNHRCISTT;
SEQ ID NO: 57
MKPFTVHSTLQSNPFSSPWMTPL;
SEQ ID NO: 58
MKPMSPMRLY;
SEQ ID NO: 59
MKSPSTLTTQSKTPSAYFSSFSP;
SEQ ID NO: 60
MLEIVMGMETYHMSNA;
SEQ ID NO: 61
MLFMRGNAMSGAPII;
SEQ ID NO: 62
MLGVVGWCRL;
SEQ ID NO: 63
MLGWVVGSFFMGGVWVGRSGIGGMLFEFM;
SEQ ID NO: 64
MLHNNPNPNTNYLPNWKQNTQMGLSL;
SEQ ID NO: 65
MLKPETSSDSPSA;
SEQ ID NO: 66
MLLGWRFQLGAWVGGLQ;
SEQ ID NO: 67
MLLMWWVSEPHCVVVNM;
SEQ ID NO: 68
MLLNSHRCSMSKDNHHSP;
SEQ ID NO: 69
MLMVEWWLGWLHM;
SEQ ID NO: 70
MLNWSNNLTNGTSYP;
SEQ ID NO: 71
MLPLHIYHNTMGLTHPPH;
SEQ ID NO: 72
MLQGQAHLSILFSI;
SEQ ID NO: 73
MLRSMLWNLWSKPQFHAHRP;
SEQ ID NO: 74
MLSPKPQQLNQQNCSPEHYEPQLKTQ;
SEQ ID NO: 75
MLTPGLVNFVPATAVTRLTQVN;
SEQ ID NO: 76
MLTSKYSNQPSTITHQLQLQSHPSPT;
SEQ ID NO: 77
MLVPFDRGDLEGELTGTGMLACVILL;
SEQ ID NO: 78
MMAHLGKKPVSGG;
SEQ ID NO: 79
MMCLRFRWCGLWS;
SEQ ID NO: 80
MMDPEHMNSMALKKAWVQMC;
SEQ ID NO: 81
MMFPIRLHNSPIRP;
SEQ ID NO: 82
MMGITMKKIITNAWAVTMTL;
SEQ ID NO: 83
MMGMWLVGHYVLSC;
SEQ ID NO: 84
MMKGKMKWKVKNRV;
SEQ ID NO: 85
MMKLRLTPWRLPDPPNHH;
SEQ ID NO: 86
MMLSWDDIIYGG;
SEQ ID NO: 87
MMMFVYSAMKN;
SEQ ID NO: 88
MMMTIECLHSHFPH;
SEQ ID NO: 89
MMNSDSIIPS;
SEQ ID NO: 90
MMSVWKVAVQTFNCYYYVLQALIN;
SEQ ID NO: 91
MMTHPTTPLTINTKPP;
SEQ ID NO: 92
MMTWSPWKPVATKNVEP;
SEQ ID NO: 93
MMWKTIVVFQLQEHQWPQYAKLTP;
SEQ ID NO: 94
MNCGGCLWGLVGSGYGVSSGVCVLG;
SEQ ID NO: 95
MNHIMAKGHDG;
SEQ ID NO: 96
MNHNQYYQSMLIINNHNSYSNKT;
SEQ ID NO: 97
MNNSSSTVQP;
SEQ ID NO: 98
MNPDQPHHLLLSLYTAIFSKPWW;
SEQ ID NO: 99
MPFWVEVMMEVEIWCCEIVLGNSFSSQV;
SEQ ID NO: 100
MPIIRRMSWSP;
SEQ ID NO: 101
MPIVTIMSPSWLEVEKATIFLMSFCV;
SEQ ID NO: 102
MPLRLEWVQWGVGGWPWVCC;
SEQ ID NO: 103
MPPKDKIWNLV;
SEQ ID NO: 104
MPPLCKIHCRIHLYYQSLPHNNIHVP;
SEQ ID NO: 105
MPRKCWLYPSRTN;
SEQ ID NO: 106
MPTNLPTLNST;
SEQ ID NO: 107
MPTPILIFRSPWSLYSYPT;
SEQ ID NO: 108
MQFTLATPKCYGPGA;
SEQ ID NO: 109
MQNPPHSSPHSSPLPRYSYLSPLLY;
SEQ ID NO: 110
MQPYRRYRFHPRLSMIYPTLQLM;
SEQ ID NO: 111
MQRWFFMSLVVVVVRA;
SEQ ID NO: 112
MQSGVLQSLAVTEIKYCNLL;
SEQ ID NO: 113
MQTKSLQSPQ;
SEQ ID NO: 114
MQWAILGLFVVG;
SEQ ID NO: 115
MRLLAWGNTWWQLLWNEGLFFWLELE;
SEQ ID NO: 116
MSGVRKEVSLL;
SEQ ID NO: 117
MSKKIPATTMVLEWS;
SEQ ID NO: 118
MSLSYLQDSTY;
SEQ ID NO: 119
MSLTPHILPTMP;
SEQ ID NO: 120
MSMPVLGWQWG;
SEQ ID NO: 121
MSNMKTFSSA;
SEQ ID NO: 122
MSNNMAFSTFKG;
SEQ ID NO: 123
MSRNHSFCLNYMPIRFSLILFVVTH;
SEQ ID NO: 124
MSSAWIIWFRLFSI;
SEQ ID NO: 125
MSSDEFANTMPV;
SEQ ID NO: 126
MSTHLPNYHTNLSYR;
SEQ ID NO: 127
MSTINQQVIITLTVNPTQACS;
SEQ ID NO: 128
MSWSNIEVVNPIVDMDS;
SEQ ID NO: 129
MSYRAHTPKMLVMPFPY;
SEQ ID NO: 130
MTAPVKLQGVWM;
SEQ ID NO: 131
MTELLTPNWTNLSPY;
SEQ ID NO: 132
MTKNFHQTPPPPLLATALKHISAKPQKQ;
SEQ ID NO: 133
MTLGHNMIYLHTS;
SEQ ID NO: 134
MTNIFGVWAR;
SEQ ID NO: 135
MTNPENQNERKSVRFIHCPHNP;
SEQ ID NO: 136
MTNTLNSIHPPLP;
SEQ ID NO: 137
MTSLHNSFYSKDTSLRTPLMTP;
SEQ ID NO: 138
MTSPKIQNNNTPDHTANNQY;
SEQ ID NO: 139
MTWPTEQVTLGMTAQSYS;
SEQ ID NO: 140
MTYSPEQSQLQYMHQQTMFNQ;
SEQ ID NO: 141
MVEGWLLYLLVSMG;
SEQ ID NO: 142
MVEVGYNVVRVV;
SEQ ID NO: 143
MVGYLIPVWVLAIVCSDMLKPLS;
SEQ ID NO: 144
MVIICFEVSLISHCWVVISRLLM;
SEQ ID NO: 145
MVKPHLQNASI;
SEQ ID NO: 146
MVLEFGLVGYFLLGGGSGWV;
SEQ ID NO: 147
MVLFLLGMVG;
SEQ ID NO: 148
MVLNLRVHRLRRTNLQLLHTSPIIP;
SEQ ID NO: 149
MVLSSPPIFRMSCSLL;
SEQ ID NO: 150
MVLWWFGGSG;
SEQ ID NO: 151
MVNGRRTSVGV;
SEQ ID NO: 152
MVRMLLVQWW;
SEQ ID NO: 153
MVSMMTSMGM;
SEQ ID NO: 154
MVSSGDWYSWCE;
SEQ ID NO: 155
MVVCMVITFIWSCTKIFGA;
SEQ ID NO: 156
MVVHWMSGVGLPWLWGVGV;
SEQ ID NO: 157
MVVKGPLSEGGHPWGREGIWL;
SEQ ID NO: 158
MWEEAGRMSEGCLG;
SEQ ID NO: 159
MWGVSDMLVFLEV;
SEQ ID NO: 160
MWLGVGLGWVG;
SEQ ID NO: 161
MWLIEEYAMSDF;
SEQ ID NO: 162
MWLVLWLWML;
SEQ ID NO: 163
MWMPPTLPHIR;
SEQ ID NO: 164
MWNDLLQCSEP;
SEQ ID NO: 165
MWRVLGALWW;
SEQ ID NO: 166
MWSHPSHHSTINITNKWLL;
SEQ ID NO: 167
MWVIEEYAKILRSWVWFNPPQLPAMMDKIE;
SEQ ID NO: 168
MYCNWDCSGE;
SEQ ID NO: 169
MYLNGTCSAS;
SEQ ID NO: 170
MYNYAKAPTL;
SEQ ID NO: 171
MYRHLQQTLMKATK;
SEQ ID NO: 172
MYTNKQCSTSNYY;
SEQ ID NO: 173
MYTPTNNVQPVTTTNQRP;
SEQ ID NO: 174
MYYSDGYWGVSWGMGV;
SEQ ID NO: 175
MYEMHSGCWWVSQYLGGTQICFPMKEQ;
SEQ ID NO: 176
MKNIEAPLAW;
SEQ ID NO: 177
MRMGASIGSTTRLSTS;
SEQ ID NO: 178
MVQTVSISWASEMLVLVSFVVSV;
SEQ ID NO: 179
MSSSMMGEVASC;
SEQ ID NO: 180
MVVGSWCWSLSWVVGMR;
SEQ ID NO: 181
MCHWAGGASNTGDA;
SEQ ID NO: 182
MFAVSMMWSLE;
SEQ ID NO: 183
MMKVDATMDFT;
SEQ ID NO: 184
MASAKGCSSP;
SEQ ID NO: 185
MLVMLEVMFLVN;
SEQ ID NO: 186
MIINDEYWLVVLVMVHCPESMLLK;
SEQ ID NO: 187
AYEGCCYSCKQEDNADVSGFWVEK;
SEQ ID NO: 188
DWSNWVWGVQLYVWDFLGSGCWAW;
SEQ ID NO: 189
FEELINVWVWVYMSQWEFYDGPCN;
SEQ ID NO: 190
MYHSENSMMDHVTNNATGMNIMEK;
SEQ ID NO: 191
LGWGWPGGWVWGGGFMVEERWWEL;
SEQ ID NO: 192
LGWVVGSFFMGGVWVGRSGIGGML;
SEQ ID NO: 193
LHNNPNPNTNYLPNWKQNTQMGLS;
SEQ ID NO: 194
LSPKPQQLNQQNCSPEHYEPQLKT;
SEQ ID NO: 195
LTSKYSNQPSTITHQLQLQSHPSP;
SEQ ID NO: 196
LVPFDRGDLEGELTGTGMLACVIL;
SEQ ID NO: 197
WVEVMMEVEIWCCEIVLGNSFSSQ;
SEQ ID NO: 198
PIVTIMSPSWLEVEKATIFLMSFC;
SEQ ID NO: 199
PLCKIHCRIHLYYQSLPHNNIHVP;
SEQ ID NO: 200
RLLAWGNTWWQLLWNEGLFFWLEL;
SEQ ID NO: 201
TKNFHQTPPPPLLATALKHISAKP;
SEQ ID NO: 202
MWVIEEYAKILRSWVWFNPPQLPA;
SEQ ID NO: 203
KNIEAPLAW;
SEQ ID NO: 204
RMGASIGSTTRLSTS;
SEQ ID NO: 205
QTVSISWASEMLVLVSFVVSV;
SEQ ID NO: 206
SSMMGEVASC;
SEQ ID NO: 207
VVGSWCWSLSWVVGMR;
SEQ ID NO: 208
CHWAGGASNTGDA;
SEQ ID NO: 209
FAVSMMWSLE;
and
SEQ ID NO: 210
MKVDATMDFT;

or pharmaceutically acceptable salts thereof.

2. An isolated peptide comprising an amino acid sequence having at least about 80% sequence identity, as measured by BLAST, with a peptide according to embodiment 1.

3. An isolated peptide comprising an amino acid sequence having at least about 90% sequence identity, as measured by BLAST, with a peptide according to embodiment 1.

4. A peptide comprising a peptide selected from

SEQ ID NO: 1
MAAAPSGCPDPTS;
SEQ ID NO: 2
MAASVERGFIFLV;
SEQ ID NO: 3
MAGGFMLMIVVMKLMAPKMEETPA;
SEQ ID NO: 4
MAHTFPLNKTSR;
SEQ ID NO: 5
MAHYSQIPSRPHGWPPSD;
SEQ ID NO: 6
MAIAVVYPKTTIIPPK;
SEQ ID NO: 7
MAIMIINDEYWLVVLVMVHCPESMLLK;
SEQ ID NO: 8
MAPPLPPLEPTVKLT;
SEQ ID NO: 9
MAQTMPMYPNGSFFPE;
SEQ ID NO: 10
MATKKNFMEKGTRAGDMGSKPHS;
SEQ ID NO: 11
MAVVDGWVNTWVVPKSASPWKN;
SEQ ID NO: 12
MAYEGCCYSCKQEDNADVSGFWVEKWSVM;
SEQ ID NO: 13
MCSCVYVYSYCKYMVCSHDKP;
SEQ ID NO: 14
MDGCALLAVL;
SEQ ID NO: 15
MDMVPQGKDEKL;
SEQ ID NO: 16
MDSWVVGVNEWG;
SEQ ID NO: 17
MDWSNWVWGVQLYVWDFLGSGCWAWTLS;
SEQ ID NO: 18
MEAMGLAWNQLWGVRFLPFLS;
SEQ ID NO: 19
METDLDYSGLNSDHVGL;
SEQ ID NO: 20
MFDQENVVTSTESSPS;
SEQ ID NO: 21
MFEELINVWVWVYMSQWEFYDGPCNEQCY;
SEQ ID NO: 22
MFGSEFMYHSENSMMDHVTNNATGMNIMEK;
SEQ ID NO: 23
MFHLRYHNHRYPHRRQSI;
SEQ ID NO: 24
MFKLSFYFYVV;
SEQ ID NO: 25
MFLVGWWGMV;
SEQ ID NO: 26
MGACQGGSDESN;
SEQ ID NO: 27
MGCDDNVVDVVVT;
SEQ ID NO: 28
MGCSGSSVSQCYRVHTPQTKMPNAW;
SEQ ID NO: 29
MGDCAVCDA;
SEQ ID NO: 30
MGEGLEENPTNPITKPTLN;
SEQ ID NO: 31
MGEVGLLWLGRI;
SEQ ID NO: 32
MGGEWGMGVWTWGCFLVWM;
SEQ ID NO: 33
MGGGVLLGLQ;
SEQ ID NO: 34
MGGLGWGWPGGWVWGGGFMVEERWWEL;
SEQ ID NO: 35
MGGRWGLGWWRAG;
SEQ ID NO: 36
MGLAAVCVCWVGWAGVVLM;
SEQ ID NO: 37
MGLLEWLLCWHLLGRIINWWA;
SEQ ID NO: 38
MGLWLVCWVL;
SEQ ID NO: 39
MGNIIRAWCGEGCL;
SEQ ID NO: 40
MGSPPPAGSKKVVL;
SEQ ID NO: 41
MGSSRINPDPSPS;
SEQ ID NO: 42
MGSSRLAVLCPPLHGQVNFTG;
SEQ ID NO: 43
MGVGMEWGLLLRRGR;
SEQ ID NO: 44
MGVKFFTLST;
SEQ ID NO: 45
MHGELPWVVN;
SEQ ID NO: 46
MHHMKHPIIC;
SEQ ID NO: 47
MHKHYYNKHPHHYNLP;
SEQ ID NO: 48
MHKRLSPIYYSHRYLPDKRL;
SEQ ID NO: 49
MHLVFSSGGYARDSIA;
SEQ ID NO: 50
MHQSCKPEMKTFFQGQI;
SEQ ID NO: 51
MIEMGASFCHV;
SEQ ID NO: 52
MITVPAHKGMAMA;
SEQ ID NO: 53
MIYVSSDVGDWWKGGWGVWWVVHG;
SEQ ID NO: 54
MKFEIWLGWC;
SEQ ID NO: 55
MKHRQVLWVLSCGS;
SEQ ID NO: 56
MKNHRCISTT;
SEQ ID NO: 57
MKPFTVHSTLQSNPFSSPWMTPL;
SEQ ID NO: 58
MKPMSPMRLY;
SEQ ID NO: 59
MKSPSTLTTQSKTPSAYFSSFSP;
SEQ ID NO: 60
MLEIVMGMETYHMSNA;
SEQ ID NO: 61
MLFMRGNAMSGAPII;
SEQ ID NO: 62
MLGVVGWCRL;
SEQ ID NO: 63
MLGWVVGSFFMGGVWVGRSGIGGMLFEFM;
SEQ ID NO: 64
MLHNNPNPNTNYLPNWKQNTQMGLSL;
SEQ ID NO: 65
MLKPETSSDSPSA;
SEQ ID NO: 66
MLLGWRFQLGAWVGGLQ;
SEQ ID NO: 67
MLLMWWVSEPHCVVVNM;
SEQ ID NO: 68
MLLNSHRCSMSKDNHHSP;
SEQ ID NO: 69
MLMVEWWLGWLHM;
SEQ ID NO: 70
MLNWSNNLTNGTSYP;
SEQ ID NO: 71
MLPLHIYHNTMGLTHPPH;
SEQ ID NO: 72
MLQGQAHLSILFSI;
SEQ ID NO: 73
MLRSMLWNLWSKPQFHAHRP;
SEQ ID NO: 74
MLSPKPQQLNQQNCSPEHYEPQLKTQ;
SEQ ID NO: 75
MLTPGLVNFVPATAVTRLTQVN;
SEQ ID NO: 76
MLTSKYSNQPSTITHQLQLQSHPSPT;
SEQ ID NO: 77
MLVPFDRGDLEGELTGTGMLACVILL;
SEQ ID NO: 78
MMAHLGKKPVSGG;
SEQ ID NO: 79
MMCLRFRWCGLWS;
SEQ ID NO: 80
MMDPEHMNSMALKKAWVQMC;
SEQ ID NO: 81
MMFPIRLHNSPIRP;
SEQ ID NO: 82
MMGITMKKIITNAWAVTMTL;
SEQ ID NO: 83
MMGMWLVGHYVLSC;
SEQ ID NO: 84
MMKGKMKWKVKNRV;
SEQ ID NO: 85
MMKLRLTPWRLPDPPNHH;
SEQ ID NO: 86
MMLSWDDIIYGG;
SEQ ID NO: 87
MMMFVYSAMKN;
SEQ ID NO: 88
MMMTIECLHSHFPH;
SEQ ID NO: 89
MMNSDSIIPS;
SEQ ID NO: 90
MMSVWKVAVQTFNCYYYVLQALIN;
SEQ ID NO: 91
MMTHPTTPLTINTKPP;
SEQ ID NO: 92
MMTWSPWKPVATKNVEP;
SEQ ID NO: 93
MMWKTIVVFQLQEHQWPQYAKLTP;
SEQ ID NO: 94
MNCGGCLWGLVGSGYGVSSGVCVLG;
SEQ ID NO: 95
MNHIMAKGHDG;
SEQ ID NO: 96
MNHNQYYQSMLIINNHNSYSNKT;
SEQ ID NO: 97
MNNSSSTVQP;
SEQ ID NO: 98
MNPDQPHHLLLSLYTAIFSKPWW;
SEQ ID NO: 99
MPFWVEVMMEVEIWCCEIVLGNSFSSQV;
SEQ ID NO: 100
MPIIRRMSWSP;
SEQ ID NO: 101
MPIVTIMSPSWLEVEKATIFLMSFCV;
SEQ ID NO: 102
MPLRLEWVQWGVGGWPWVCC;
SEQ ID NO: 103
MPPKDKIWNLV;
SEQ ID NO: 104
MPPLCKIHCRIHLYYQSLPHNNIHVP;
SEQ ID NO: 105
MPRKCWLYPSRTN;
SEQ ID NO: 106
MPTNLPTLNST;
SEQ ID NO: 107
MPTPILIFRSPWSLYSYPT;
SEQ ID NO: 108
MQFTLATPKCYGPGA;
SEQ ID NO: 109
MQNPPHSSPHSSPLPRYSYLSPLLY;
SEQ ID NO: 110
MQPYRRYRFHPRLSMIYPTLQLM;
SEQ ID NO: 111
MQRWFFMSLVVVVVRA;
SEQ ID NO: 112
MQSGVLQSLAVTEIKYCNLL;
SEQ ID NO: 113
MQTKSLQSPQ;
SEQ ID NO: 114
MQWAILGLFVVG;
SEQ ID NO: 115
MRLLAWGNTWWQLLWNEGLFFWLELE;
SEQ ID NO: 116
MSGVRKEVSLL;
SEQ ID NO: 117
MSKKIPATTMVLEWS;
SEQ ID NO: 118
MSLSYLQDSTY;
SEQ ID NO: 119
MSLTPHILPTMP;
SEQ ID NO: 120
MSMPVLGWQWG;
SEQ ID NO: 121
MSNMKTFSSA;
SEQ ID NO: 122
MSNNMAFSTFKG;
SEQ ID NO: 123
MSRNHSFCLNYMPIRFSLILFVVTH;
SEQ ID NO: 124
MSSAWIIWFRLFSI;
SEQ ID NO: 125
MSSDEFANTMPV;
SEQ ID NO: 126
MSTHLPNYHTNLSYR;
SEQ ID NO: 127
MSTINQQVIITLTVNPTQACS;
SEQ ID NO: 128
MSWSNIEVVNPIVDMDS;
SEQ ID NO: 129
MSYRAHTPKMLVMPFPY;
SEQ ID NO: 130
MTAPVKLQGVWM;
SEQ ID NO: 131
MTELLTPNWTNLSPY;
SEQ ID NO: 132
MTKNFHQTPPPPLLATALKHISAKPQKQ;
SEQ ID NO: 133
MTLGHNMIYLHTS;
SEQ ID NO: 134
MTNIFGVWAR;
SEQ ID NO: 135
MTNPENQNERKSVRFIHCPHNP;
SEQ ID NO: 136
MTNTLNSIHPPLP;
SEQ ID NO: 137
MTSLHNSFYSKDTSLRTPLMTP;
SEQ ID NO: 138
MTSPKIQNNNTPDHTANNQY;
SEQ ID NO: 139
MTWPTEQVTLGMTAQSYS;
SEQ ID NO: 140
MTYSPEQSQLQYMHQQTMFNQ;
SEQ ID NO: 141
MVEGWLLYLLVSMG;
SEQ ID NO: 142
MVEVGYNVVRVV;
SEQ ID NO: 143
MVGYLIPVWVLAIVCSDMLKPLS;
SEQ ID NO: 144
MVIICFEVSLISHCWVVISRLLM;
SEQ ID NO: 145
MVKPHLQNASI;
SEQ ID NO: 146
MVLEFGLVGYFLLGGGSGWV;
SEQ ID NO: 147
MVLFLLGMVG;
SEQ ID NO: 148
MVLNLRVHRLRRTNLQLLHTSPIIP;
SEQ ID NO: 149
MVLSSPPIFRMSCSLL;
SEQ ID NO: 150
MVLWWFGGSG;
SEQ ID NO: 151
MVNGRRTSVGV;
SEQ ID NO: 152
MVRMLLVQWW;
SEQ ID NO: 153
MVSMMTSMGM;
SEQ ID NO: 154
MVSSGDWYSWCE;
SEQ ID NO: 155
MVVCMVITFIWSCTKIFGA;
SEQ ID NO: 156
MVVHWMSGVGLPWLWGVGV;
SEQ ID NO: 157
MVVKGPLSEGGHPWGREGIWL;
SEQ ID NO: 158
MWEEAGRMSEGCLG;
SEQ ID NO: 159
MWGVSDMLVFLEV;
SEQ ID NO: 160
MWLGVGLGWVG;
SEQ ID NO: 161
MWLIEEYAMSDF;
SEQ ID NO: 162
MWLVLWLWML;
SEQ ID NO: 163
MWMPPTLPHIR;
SEQ ID NO: 164
MWNDLLQCSEP;
SEQ ID NO: 165
MWRVLGALWW;
SEQ ID NO: 166
MWSHPSHHSTINITNKWLL;
SEQ ID NO: 167
MWVIEEYAKILRSWVWFNPPQLPAMMDKIE;
SEQ ID NO: 168
MYCNWDCSGE;
SEQ ID NO: 169
MYLNGTCSAS;
SEQ ID NO: 170
MYNYAKAPTL;
SEQ ID NO: 171
MYRHLQQTLMKATK;
SEQ ID NO: 172
MYTNKQCSTSNYY;
SEQ ID NO: 173
MYTPTNNVQPVTTTNQRP;
SEQ ID NO: 174
MYYSDGYWGVSWGMGV;
SEQ ID NO: 175
MYEMHSGCWWVSQYLGGTQICFPMKEQ;
SEQ ID NO: 176
MKNIEAPLAW;
SEQ ID NO: 177
MRMGASIGSTTRLSTS;
SEQ ID NO: 178
MVQTVSISWASEMLVLVSFVVSV;
SEQ ID NO: 179
MSSSMMGEVASC;
SEQ ID NO: 180
MVVGSWCWSLSWVVGMR;
SEQ ID NO: 181
MCHWAGGASNTGDA;
SEQ ID NO: 182
MFAVSMMWSLE;
SEQ ID NO: 183
MMKVDATMDFT;
SEQ ID NO: 184
MASAKGCSSP;
SEQ ID NO: 185
MLVMLEVMFLVN;
SEQ ID NO: 186
MIINDEYWLVVLVMVHCPESMLLK;
SEQ ID NO: 187
AYEGCCYSCKQEDNADVSGFWVEK;
SEQ ID NO: 188
DWSNWVWGVQLYVWDFLGSGCWAW;
SEQ ID NO: 189
FEELINVWVWVYMSQWEFYDGPCN;
SEQ ID NO: 190
MYHSENSMMDHVTNNATGMNIMEK;
SEQ ID NO: 191
LGWGWPGGWVWGGGFMVEERWWEL;
SEQ ID NO: 192
LGWVVGSFFMGGVWVGRSGIGGML;
SEQ ID NO: 193
LHNNPNPNTNYLPNWKQNTQMGLS;
SEQ ID NO: 194
LSPKPQQLNQQNCSPEHYEPQLKT;
SEQ ID NO: 195
LTSKYSNQPSTITHQLQLQSHPSP;
SEQ ID NO: 196
LVPFDRGDLEGELTGTGMLACVIL;
SEQ ID NO: 197
WVEVMMEVEIWCCEIVLGNSFSSQ;
SEQ ID NO: 198
PIVTIMSPSWLEVEKATIFLMSFC;
SEQ ID NO: 199
PLCKIHCRIHLYYQSLPHNNIHVP;
SEQ ID NO: 200
RLLAWGNTWWQLLWNEGLFFWLEL;
SEQ ID NO: 201
TKNFHQTPPPPLLATALKHISAKP;
SEQ ID NO: 202
MWVIEEYAKILRSWVWFNPPQLPA;
SEQ ID NO: 203
KNIEAPLAW;
SEQ ID NO: 204
RMGASIGSTTRLSTS;
SEQ ID NO: 205
VQTVSISWASEMLVLVSFVVSV;
SEQ ID NO: 206
SSSMMGEVASC;
SEQ ID NO: 207
VVGSWCWSLSWVVGMR;
SEQ ID NO: 208
CHWAGGASNTGDA;
SEQ ID NO: 209
FAVSMMWSLE;
and
SEQ ID NO: 210
MKVDATMDFT;

wherein the amino acid sequence of the peptide is modified such that it comprises a deletion, insertion or substitution of one to four amino acids.

5. A peptide comprising any of the following amino acid sequences:

SEQ ID NO: 12
MAYEGCCYSCKQEDNADVSGFWVEKWSVM;
SEQ ID NO: 21
MFEELINVWVWVYMSQWEFYDGPCNEQCY;
SEQ ID NO: 22
MFGSEFMYHSENSMMDHVTNNATGMNIMEK;
SEQ ID NO: 36
MGLAAVCVCWVGWAGVVLM;
SEQ ID NO: 51
MIEMGASFCHV;
SEQ ID NO: 59
MKSPSTLTTQSKTPSAYFSSFSP;
SEQ ID NO: 64
MLHNNPNPNTNYLPNWKQNTQMGLSL;
SEQ ID NO: 67
MLLMWWVSEPHCVVVNM;
SEQ ID NO: 74
MLSPKPQQLNQQNCSPEHYEPQLKTQ;
SEQ ID NO: 76
MLTSKYSNQPSTITHQLQLQSHPSPT;
SEQ ID NO: 77
MLVPFDRGDLEGELTGTGMLACVILL;
SEQ ID NO: 94
MNCGGCLWGLVGSGYGVSSGVCVLG;
SEQ ID NO: 99
MPFWVEVMMEVEIWCCEIVLGNSFSSQV;
SEQ ID NO: 101
MPIVTIMSPSWLEVEKATIFLMSFCV;
SEQ ID NO: 104
MPPLCKIHCRIHLYYQSLPHNNIHVP;
SEQ ID NO: 109
MQNPPHSSPHSSPLPRYSYLSPLLY;
SEQ ID NO: 111
MQRWFFMSLVVVVVRA;
SEQ ID NO: 112
MQSGVLQSLAVTEIKYCNLL;
SEQ ID NO: 115
MRLLAWGNTWWQLLWNEGLFFWLELE;
SEQ ID NO: 122
MSNNMAFSTFKG;
SEQ ID NO: 127
MSTINQQVIITLTVNPTQACS;
SEQ ID NO: 131
MTELLTPNWTNLSPY;
SEQ ID NO: 137
MTSLHNSFYSKDTSLRTPLMTP;
SEQ ID NO: 141
MVEGWLLYLLVSMG;
SEQ ID NO: 144
MVIICFEVSLISHCWVVISRLLM;
SEQ ID NO: 148
MVLNLRVHRLRRTNLQLLHTSPIIP;
SEQ ID NO: 167
MWVIEEYAKILRSWVWFNPPQLPAMMDKIE;
and
SEQ ID NO: 171
MYRHLQQTLMKATK;

or a pharmaceutically acceptable salt thereof.

6. A peptide of any one of embodiments 1-5, wherein the peptide comprises substitution with at least one amino acid selected from (i) an amino acid having a D-configuration, and (ii) a non-naturally occurring amino acid residue; or pharmaceutically acceptable salts thereof.

7. A peptide of any one of embodiments 1-6, further comprising a duration enhancing moiety, optionally further comprising a metabolically cleavable linker coupling the peptide to the duration enhancing moiety.

8. A conjugate comprising the peptide of any one of embodiments 1-7 conjugated to a heterologous moiety.

9. A composition comprising a peptide of any of embodiments 1-7 or a conjugate of embodiment 8 and a pharmaceutically acceptable excipient.

10. The composition of embodiment 9, wherein the pharmaceutically acceptable excipient is not found in nature.

11. A method of modulating cell viability comprising administering a peptide of any one of embodiments 1-7, a conjugate of embodiment 8, or a composition of embodiment 9 or 10.

12. A method of treating cell proliferation in patient in need of such treatment, comprising administering to the patient a pharmacologically effective amount of a peptide of any one of embodiments 1-7, a conjugate of embodiment 8, or a composition of embodiment 9 or 10.

13. A method of treating an apoptosic disease in a patient in need of such treatment, comprising administering to the patient a pharmacologically effect amount of a peptide of any one of embodiments 1-7, a conjugate of embodiment 8, or a composition of embodiment 9 or 10.

14. A method of treating a metabolic disease in a patient in need of such treatment, comprising administering to the patient a pharmacologically effect amount of a peptide of any one of embodiments 1-7, a conjugate of embodiment 8, or a composition of embodiment 9 or 10.

15. A method of providing cytoprotection in a patient in need of such treatment, comprising administering to the patient a pharmacologically effect amount of a peptide of any one embodiments 1-7, a conjugate of embodiment 8, or a composition of embodiment 9 or 10.

16. A nucleic acid comprising a nucleotide sequence that encodes a peptide according to any one of embodiments 1-7.

17. A vector, preferably an expression vector, comprising the nucleic acid according to embodiment 15.

18. A host cell, preferably an isolated host cell, comprising the nucleic acid of embodiment 16 or the vector of embodiment 17.

19. A method of treating a metabolic disease in a subject in need thereof, comprising administering to the subject a peptide or peptide analog, a conjugate, or a composition of any one of embodiments 1-10, in an amount effective to treat the metabolic disease.

20. The method of embodiment 19, wherein the disease is selected from the group consisting of obesity, diabetes (e.g., Type 2 diabetes), cognitive disorders and/or neurodegenerative disorders, cardiovascular disease, fatty liver disease, and gastrointestinal disease.

21. A method of treating a cancer in a subject in need thereof, comprising administering to the subject a peptide or peptide analog, a conjugate, or a composition of any one of embodiments 1-10, in an amount effective to treat the cancer.

22. The method of embodiment 21, wherein the cancer is lung cancer, pancreatic cancer, breast cancer, prostate cancer, ovarian cancer, or hepatocellular cancer.

23. A method of treating a liver disease in a subject in need thereof, comprising administering to the subject a peptide or peptide analog, a conjugate, or a composition of any one of embodiments 1-10, in an amount effective to treat the liver disease.

24. The method of embodiment 23, wherein the liver disease is a fatty liver disease.

25. The method of embodiment 24, wherein the fatty liver disease is NAFLD or NASH.

26. A method of modulating fatty acid metabolism in a subject in need thereof, comprising administering to the subject a peptide or peptide analog, a conjugate, or a composition of any one of embodiments 1-10, in an amount effective to modulate fatty acid metabolism.

27. The method of embodiment 26, wherein fatty acid metabolism is increased in the subject after the peptide or peptide analog, conjugate, or composition is administered to the subject.

28. A method of reducing body weight in a subject in need thereof, comprising administering to the subject a peptide or peptide analog, a conjugate, or a composition of any one of embodiments 1-10, in an amount effective to reduce body weight in the subject.

29. The peptides, peptide analogs, conjugates, or compositions of any one of embodiments 1-10 for use in a therapeutic treatment of a metabolic disease, cancer, liver disease, or any disease, disorder, or medical condition described herein.

30. Use of any one of the peptides, peptide analogs, conjugates, or compositions of any one of embodiments 1-10 in therapeutic treatment of a metabolic disease, cancer, liver disease, or any disease, disorder, or medical condition described herein.

31. Use of any one of the peptides, peptide analogs, conjugates, or compositions of any one embodiments 1-10 in the manufacture of a medicament for treating a metabolic disease, cancer, liver disease, or any disease, disorder, or medical condition described herein.

Relatedly, the embodiments listed below are presented in numbered form for convenience and for ease and clarity of reference in referring back to multiple embodiments. The invention provides:

1A. A peptide comprising any of the following amino acid sequences:

SEQ ID NO: 1
MAAAPSGCPDPTS;
SEQ ID NO: 2
MAASVERGFIFLV;
SEQ ID NO: 3
MAGGFMLMIVVMKLMAPKMEETPA;
SEQ ID NO: 4
MAHTFPLNKTSR;
SEQ ID NO: 5
MAHYSQIPSRPHGWPPSD;
SEQ ID NO: 6
MAIAVVYPKTTIIPPK;
SEQ ID NO: 7
MAIMIINDEYWLVVLVMVHCPESMLLK;
SEQ ID NO: 8
MAPPLPPLEPTVKLT;
SEQ ID NO: 9
MAQTMPMYPNGSFFPE;
SEQ ID NO: 10
MATKKNFMEKGTRAGDMGSKPHS;
SEQ ID NO: 11
MAVVDGWVNTWVVPKSASPWKN;
SEQ ID NO: 12
MAYEGCCYSCKQEDNADVSGFWVEKWSVM;
SEQ ID NO: 13
MCSCVYVYSYCKYMVCSHDKP;
SEQ ID NO: 14
MDGCALLAVL;
SEQ ID NO: 15
MDMVPQGKDEKL;
SEQ ID NO: 16
MDSWVVGVNEWG;
SEQ ID NO: 17
MDWSNWVWGVQLYVWDFLGSGCWAWTLS;
SEQ ID NO: 18
MEAMGLAWNQLWGVRFLPFLS;
SEQ ID NO: 19
METDLDYSGLNSDHVGL;
SEQ ID NO: 20
MFDQENVVTSTESSPS;
SEQ ID NO: 21
MFEELINVWVWVYMSQWEFYDGPCNEQCY;
SEQ ID NO: 22
MFGSEFMYHSENSMMDHVTNNATGMNIMEK;
SEQ ID NO: 23
MFHLRYHNHRYPHRRQSI;
SEQ ID NO: 24
MFKLSFYFYVV;
SEQ ID NO: 25
MFLVGWWGMV;
SEQ ID NO: 26
MGACQGGSDESN;
SEQ ID NO: 27
MGCDDNVVDVVVT;
SEQ ID NO: 28
MGCSGSSVSQCYRVHTPQTKMPNAW;
SEQ ID NO: 29
MGDCAVCDA;
SEQ ID NO: 30
MGEGLEENPTNPITKPTLN;
SEQ ID NO: 31
MGEVGLLWLGRI;
SEQ ID NO: 32
MGGEWGMGVWTWGCFLVWM;
SEQ ID NO: 33
MGGGVLLGLQ;
SEQ ID NO: 34
MGGLGWGWPGGWVWGGGFMVEERWWEL;
SEQ ID NO: 35
MGGRWGLGWWRAG;
SEQ ID NO: 36
MGLAAVCVCWVGWAGVVLM;
SEQ ID NO: 37
MGLLEWLLCWHLLGRIINWWA;
SEQ ID NO: 38
MGLWLVCWVL;
SEQ ID NO: 39
MGNIIRAWCGEGCL;
SEQ ID NO: 40
MGSPPPAGSKKVVL;
SEQ ID NO: 41
MGSSRINPDPSPS;
SEQ ID NO: 42
MGSSRLAVLCPPLHGQVNFTG;
SEQ ID NO: 43
MGVGMEWGLLLRRGR;
SEQ ID NO: 44
MGVKFFTLST;
SEQ ID NO: 45
MHGELPWVVN;
SEQ ID NO: 46
MHHMKHPIIC;
SEQ ID NO: 47
MHKHYYNKHPHHYNLP;
SEQ ID NO: 48
MHKRLSPIYYSHRYLPDKRL;
SEQ ID NO: 49
MHLVFSSGGYARDSIA;
SEQ ID NO: 50
MHQSCKPEMKTFFQGQI;
SEQ ID NO: 51
MIEMGASFCHV;
SEQ ID NO: 52
MITVPAHKGMAMA;
SEQ ID NO: 53
MIYVSSDVGDWWKGGWGVWWVVHG;
SEQ ID NO: 54
MKFEIWLGWC;
SEQ ID NO: 55
MKHRQVLWVLSCGS;
SEQ ID NO: 56
MKNHRCISTT;
SEQ ID NO: 57
MKPFTVHSTLQSNPFSSPWMTPL;
SEQ ID NO: 58
MKPMSPMRLY;
SEQ ID NO: 59
MKSPSTLTTQSKTPSAYFSSFSP;
SEQ ID NO: 60
MLEIVMGMETYHMSNA;
SEQ ID NO: 61
MLFMRGNAMSGAPII;
SEQ ID NO: 62
MLGVVGWCRL;
SEQ ID NO: 63
MLGWVVGSFFMGGVWVGRSGIGGMLFEFM;
SEQ ID NO: 64
MLHNNPNPNTNYLPNWKQNTQMGLSL;
SEQ ID NO: 65
MLKPETSSDSPSA;
SEQ ID NO: 66
MLLGWRFQLGAWVGGLQ;
SEQ ID NO: 67
MLLMWWVSEPHCVVVNM;
SEQ ID NO: 68
MLLNSHRCSMSKDNHHSP;
SEQ ID NO: 69
MLMVEWWLGWLHM;
SEQ ID NO: 70
MLNWSNNLTNGTSYP;
SEQ ID NO: 71
MLPLHIYHNTMGLTHPPH;
SEQ ID NO: 72
MLQGQAHLSILFSI;
SEQ ID NO: 73
MLRSMLWNLWSKPQFHAHRP;
SEQ ID NO: 74
MLSPKPQQLNQQNCSPEHYEPQLKTQ;
SEQ ID NO: 75
MLTPGLVNFVPATAVTRLTQVN;
SEQ ID NO: 76
MLTSKYSNQPSTITHQLQLQSHPSPT;
SEQ ID NO: 77
MLVPFDRGDLEGELTGTGMLACVILL;
SEQ ID NO: 78
MMAHLGKKPVSGG;
SEQ ID NO: 79
MMCLRFRWCGLWS;
SEQ ID NO: 80
MMDPEHMNSMALKKAWVQMC;
SEQ ID NO: 81
MMFPIRLHNSPIRP;
SEQ ID NO: 82
MMGITMKKIITNAWAVTMTL;
SEQ ID NO: 83
MMGMWLVGHYVLSC;
SEQ ID NO: 84
MMKGKMKWKVKNRV;
SEQ ID NO: 85
MMKLRLTPWRLPDPPNHH;
SEQ ID NO: 86
MMLSWDDIIYGG;
SEQ ID NO: 87
MMMFVYSAMKN;
SEQ ID NO: 88
MMMTIECLHSHFPH;
SEQ ID NO: 89
MMNSDSIIPS;
SEQ ID NO: 90
MMSVWKVAVQTFNCYYYVLQALIN;
SEQ ID NO: 91
MMTHPTTPLTINTKPP;
SEQ ID NO: 92
MMTWSPWKPVATKNVEP;
SEQ ID NO: 93
MMWKTIVVFQLQEHQWPQYAKLTP;
SEQ ID NO: 94
MNCGGCLWGLVGSGYGVSSGVCVLG;
SEQ ID NO: 95
MNHIMAKGHDG;
SEQ ID NO: 96
MNHNQYYQSMLIINNHNSYSNKT;
SEQ ID NO: 97
MNNSSSTVQP;
SEQ ID NO: 98
MNPDQPHHLLLSLYTAIFSKPWW;
SEQ ID NO: 99
MPFWVEVMMEVEIWCCEIVLGNSFSSQV;
SEQ ID NO: 100
MPIIRRMSWSP;
SEQ ID NO: 101
MPIVTIMSPSWLEVEKATIFLMSFCV;
SEQ ID NO: 102
MPLRLEWVQWGVGGWPWVCC;
SEQ ID NO: 103
MPPKDKIWNLV;
SEQ ID NO: 104
MPPLCKIHCRIHLYYQSLPHNNIHVP;
SEQ ID NO: 105
MPRKCWLYPSRTN;
SEQ ID NO: 106
MPTNLPTLNST;
SEQ ID NO: 107
MPTPILIFRSPWSLYSYPT;
SEQ ID NO: 108
MQFTLATPKCYGPGA;
SEQ ID NO: 109
MQNPPHSSPHSSPLPRYSYLSPLLY;
SEQ ID NO: 110
MQPYRRYRFHPRLSMIYPTLQLM;
SEQ ID NO: 111
MQRWFFMSLVVVVVRA;
SEQ ID NO: 112
MQSGVLQSLAVTEIKYCNLL;
SEQ ID NO: 113
MQTKSLQSPQ;
SEQ ID NO: 114
MQWAILGLFVVG;
SEQ ID NO: 115
MRLLAWGNTWWQLLWNEGLFFWLELE;
SEQ ID NO: 116
MSGVRKEVSLL;
SEQ ID NO: 117
MSKKIPATTMVLEWS;
SEQ ID NO: 118
MSLSYLQDSTY;
SEQ ID NO: 119
MSLTPHILPTMP;
SEQ ID NO: 120
MSMPVLGWQWG;
SEQ ID NO: 121
MSNMKTFSSA;
SEQ ID NO: 122
MSNNMAFSTFKG;
SEQ ID NO: 123
MSRNHSFCLNYMPIRFSLILFVVTH;
SEQ ID NO: 124
MSSAWIIWFRLFSI;
SEQ ID NO: 125
MSSDEFANTMPV;
SEQ ID NO: 126
MSTHLPNYHTNLSYR;
SEQ ID NO: 127
MSTINQQVIITLTVNPTQACS;
SEQ ID NO: 128
MSWSNIEVVNPIVDMDS;
SEQ ID NO: 129
MSYRAHTPKMLVMPFPY;
SEQ ID NO: 130
MTAPVKLQGVWM;
SEQ ID NO: 131
MTELLTPNWTNLSPY;
SEQ ID NO: 132
MTKNFHQTPPPPLLATALKHISAKPQKQ;
SEQ ID NO: 133
MTLGHNMIYLHTS;
SEQ ID NO: 134
MTNIFGVWAR;
SEQ ID NO: 135
MTNPENQNERKSVRFIHCPHNP;
SEQ ID NO: 136
MTNTLNSIHPPLP;
SEQ ID NO: 137
MTSLHNSFYSKDTSLRTPLMTP;
SEQ ID NO: 138
MTSPKIQNNNTPDHTANNQY;
SEQ ID NO: 139
MTWPTEQVTLGMTAQSYS;
SEQ ID NO: 140
MTYSPEQSQLQYMHQQTMFNQ;
SEQ ID NO: 141
MVEGWLLYLLVSMG;
SEQ ID NO: 142
MVEVGYNVVRVV;
SEQ ID NO: 143
MVGYLIPVWVLAIVCSDMLKPLS;
SEQ ID NO: 144
MVIICFEVSLISHCWVVISRLLM;
SEQ ID NO: 145
MVKPHLQNASI;
SEQ ID NO: 146
MVLEFGLVGYFLLGGGSGWV;
SEQ ID NO: 147
MVLFLLGMVG;
SEQ ID NO: 148
MVLNLRVHRLRRTNLQLLHTSPIIP;
SEQ ID NO: 149
MVLSSPPIFRMSCSLL;
SEQ ID NO: 150
MVLWWFGGSG;
SEQ ID NO: 151
MVNGRRTSVGV;
SEQ ID NO: 152
MVRMLLVQWW;
SEQ ID NO: 153
MVSMMTSMGM;
SEQ ID NO: 154
MVSSGDWYSWCE;
SEQ ID NO: 155
MVVCMVITFIWSCTKIFGA;
SEQ ID NO: 156
MVVHWMSGVGLPWLWGVGV;
SEQ ID NO: 157
MVVKGPLSEGGHPWGREGIWL;
SEQ ID NO: 158
MWEEAGRMSEGCLG;
SEQ ID NO: 159
MWGVSDMLVFLEV;
SEQ ID NO: 160
MWLGVGLGWVG;
SEQ ID NO: 161
MWLIEEYAMSDF;
SEQ ID NO: 162
MWLVLWLWML;
SEQ ID NO: 163
MWMPPTLPHIR;
SEQ ID NO: 164
MWNDLLQCSEP;
SEQ ID NO: 165
MWRVLGALWW;
SEQ ID NO: 166
MWSHPSHHSTINITNKWLL;
SEQ ID NO: 167
MWVIEEYAKILRSWVWFNPPQLPAMMDKIE;
SEQ ID NO: 168
MYCNWDCSGE;
SEQ ID NO: 169
MYLNGTCSAS;
SEQ ID NO: 170
MYNYAKAPTL;
SEQ ID NO: 171
MYRHLQQTLMKATK;
SEQ ID NO: 172
MYTNKQCSTSNYY;
SEQ ID NO: 173
MYTPTNNVQPVTTTNQRP;
SEQ ID NO: 174
MYYSDGYWGVSWGMGV;
SEQ ID NO: 175
MYEMHSGCWWVSQYLGGTQICFPMKEQ;
SEQ ID NO: 176
MKNIEAPLAW;
SEQ ID NO: 177
MRMGASIGSTTRLSTS;
SEQ ID NO: 178
MVQTVSISWASEMLVLVSFVVSV;
SEQ ID NO: 179
MSSSMMGEVASC;
SEQ ID NO: 180
MVVGSWCWSLSWVVGMR;
SEQ ID NO: 181
MCHWAGGASNTGDA;
SEQ ID NO: 182
MFAVSMMWSLE;
SEQ ID NO: 183
MMKVDATMDFT;
SEQ ID NO: 184
MASAKGCSSP;
and
SEQ ID NO: 185
MLVMLEVMFLVN;

or pharmaceutically acceptable salts thereof.

2A. An isolated peptide comprising an amino acid sequence having at least about 70% sequence identity, as measured by BLAST, with a peptide according to paragraph 1A.

3A. An isolated peptide comprising an amino acid sequence having at least about 80% sequence identity, as measured by BLAST, with a peptide according to paragraph 1A.

4A. An isolated peptide comprising an amino acid sequence having at least about 90% sequence identity, as measured by BLAST, with a peptide according to paragraph 1A.

5A. A peptide comprising an amino acid sequence having a deletion, insertion or substitution of one to four amino acids compared to a reference peptide that comprises an amino acid sequence selected from

SEQ ID NO: 1
MAAAPSGCPDPTS;
SEQ ID NO: 2
MAASVERGFIFLV;
SEQ ID NO: 3
MAGGFMLMIVVMKLMAPKMEETPA;
SEQ ID NO: 4
MAHTFPLNKTSR;
SEQ ID NO: 5
MAHYSQIPSRPHGWPPSD;
SEQ ID NO: 6
MAIAVVYPKTTIIPPK;
SEQ ID NO: 7
MAIMIINDEYWLVVLVMVHCPESMLLK;
SEQ ID NO: 8
MAPPLPPLEPTVKLT;
SEQ ID NO: 9
MAQTMPMYPNGSFFPE;
SEQ ID NO: 10
MATKKNFMEKGTRAGDMGSKPHS;
SEQ ID NO: 11
MAVVDGWVNTWVVPKSASPWKN;
SEQ ID NO: 12
MAYEGCCYSCKQEDNADVSGFWVEKWSVM;
SEQ ID NO: 13
MCSCVYVYSYCKYMVCSHDKP;
SEQ ID NO: 14
MDGCALLAVL;
SEQ ID NO: 15
MDMVPQGKDEKL;
SEQ ID NO: 16
MDSWVVGVNEWG;
SEQ ID NO: 17
MDWSNWVWGVQLYVWDFLGSGCWAWTLS;
SEQ ID NO: 18
MEAMGLAWNQLWGVRFLPFLS;
SEQ ID NO: 19
METDLDYSGLNSDHVGL;
SEQ ID NO: 20
MFDQENVVTSTESSPS;
SEQ ID NO: 21
MFEELINVWVWVYMSQWEFYDGPCNEQCY;
SEQ ID NO: 22
MFGSEFMYHSENSMMDHVTNNATGMNIMEK;
SEQ ID NO: 23
MFHLRYHNHRYPHRRQSI;
SEQ ID NO: 24
MFKLSFYFYVV;
SEQ ID NO: 25
MFLVGWWGMV;
SEQ ID NO: 26
MGACQGGSDESN;
SEQ ID NO: 27
MGCDDNVVDVVVT;
SEQ ID NO: 28
MGCSGSSVSQCYRVHTPQTKMPNAW;
SEQ ID NO: 29
MGDCAVCDA;
SEQ ID NO: 30
MGEGLEENPTNPITKPTLN;
SEQ ID NO: 31
MGEVGLLWLGRI;
SEQ ID NO: 32
MGGEWGMGVWTWGCFLVWM;
SEQ ID NO: 33
MGGGVLLGLQ;
SEQ ID NO: 34
MGGLGWGWPGGWVWGGGFMVEERWWEL;
SEQ ID NO: 35
MGGRWGLGWWRAG;
SEQ ID NO: 36
MGLAAVCVCWVGWAGVVLM;
SEQ ID NO: 37
MGLLEWLLCWHLLGRIINWWA;
SEQ ID NO: 38
MGLWLVCWVL;
SEQ ID NO: 39
MGNIIRAWCGEGCL;
SEQ ID NO: 40
MGSPPPAGSKKVVL;
SEQ ID NO: 41
MGSSRINPDPSPS;
SEQ ID NO: 42
MGSSRLAVLCPPLHGQVNFTG;
SEQ ID NO: 43
MGVGMEWGLLLRRGR;
SEQ ID NO: 44
MGVKFFTLST;
SEQ ID NO: 45
MHGELPWVVN;
SEQ ID NO: 46
MHHMKHPIIC;
SEQ ID NO: 47
MHKHYYNKHPHHYNLP;
SEQ ID NO: 48
MHKRLSPIYYSHRYLPDKRL;
SEQ ID NO: 49
MHLVFSSGGYARDSIA;
SEQ ID NO: 50
MHQSCKPEMKTFFQGQI;
SEQ ID NO: 51
MIEMGASFCHV;
SEQ ID NO: 52
MITVPAHKGMAMA;
SEQ ID NO: 53
MIYVSSDVGDWWKGGWGVWWVVHG;
SEQ ID NO: 54
MKFEIWLGWC;
SEQ ID NO: 55
MKHRQVLWVLSCGS;
SEQ ID NO: 56
MKNHRCISTT;
SEQ ID NO: 57
MKPFTVHSTLQSNPFSSPWMTPL;
SEQ ID NO: 58
MKPMSPMRLY;
SEQ ID NO: 59
MKSPSTLTTQSKTPSAYFSSFSP;
SEQ ID NO: 60
MLEIVMGMETYHMSNA;
SEQ ID NO: 61
MLFMRGNAMSGAPII;
SEQ ID NO: 62
MLGVVGWCRL;
SEQ ID NO: 63
MLGWVVGSFFMGGVWVGRSGIGGMLFEFM;
SEQ ID NO: 64
MLHNNPNPNTNYLPNWKQNTQMGLSL;
SEQ ID NO: 65
MLKPETSSDSPSA;
SEQ ID NO: 66
MLLGWRFQLGAWVGGLQ;
SEQ ID NO: 67
MLLMWWVSEPHCVVVNM;
SEQ ID NO: 68
MLLNSHRCSMSKDNHHSP;
SEQ ID NO: 69
MLMVEWWLGWLHM;
SEQ ID NO: 70
MLNWSNNLTNGTSYP;
SEQ ID NO: 71
MLPLHIYHNTMGLTHPPH;
SEQ ID NO: 72
MLQGQAHLSILFSI;
SEQ ID NO: 73
MLRSMLWNLWSKPQFHAHRP;
SEQ ID NO: 74
MLSPKPQQLNQQNCSPEHYEPQLKTQ;
SEQ ID NO: 75
MLTPGLVNFVPATAVTRLTQVN;
SEQ ID NO: 76
MLTSKYSNQPSTITHQLQLQSHPSPT;
SEQ ID NO: 77
MLVPFDRGDLEGELTGTGMLACVILL;
SEQ ID NO: 78
MMAHLGKKPVSGG;
SEQ ID NO: 79
MMCLRFRWCGLWS;
SEQ ID NO: 80
MMDPEHMNSMALKKAWVQMC;
SEQ ID NO: 81
MMFPIRLHNSPIRP;
SEQ ID NO: 82
MMGITMKKIITNAWAVTMTL;
SEQ ID NO: 83
MMGMWLVGHYVLSC;
SEQ ID NO: 84
MMKGKMKWKVKNRV;
SEQ ID NO: 85
MMKLRLTPWRLPDPPNHH;
SEQ ID NO: 86
MMLSWDDIIYGG;
SEQ ID NO: 87
MMMFVYSAMKN;
SEQ ID NO: 88
MMMTIECLHSHFPH;
SEQ ID NO: 89
MMNSDSIIPS;
SEQ ID NO: 90
MMSVWKVAVQTFNCYYYVLQALIN;
SEQ ID NO: 91
MMTHPTTPLTINTKPP;
SEQ ID NO: 92
MMTWSPWKPVATKNVEP;
SEQ ID NO: 93
MMWKTIVVFQLQEHQWPQYAKLTP;
SEQ ID NO: 94
MNCGGCLWGLVGSGYGVSSGVCVLG;
SEQ ID NO: 95
MNHIMAKGHDG;
SEQ ID NO: 96
MNHNQYYQSMLIINNHNSYSNKT;
SEQ ID NO: 97
MNNSSSTVQP;
SEQ ID NO: 98
MNPDQPHHLLLSLYTAIFSKPWW;
SEQ ID NO: 99
MPFWVEVMMEVEIWCCEIVLGNSFSSQV;
SEQ ID NO: 100
MPIIRRMSWSP;
SEQ ID NO: 101
MPIVTIMSPSWLEVEKATIFLMSFCV;
SEQ ID NO: 102
MPLRLEWVQWGVGGWPWVCC;
SEQ ID NO: 103
MPPKDKIWNLV;
SEQ ID NO: 104
MPPLCKIHCRIHLYYQSLPHNNIHVP;
SEQ ID NO: 105
MPRKCWLYPSRTN;
SEQ ID NO: 106
MPTNLPTLNST;
SEQ ID NO: 107
MPTPILIFRSPWSLYSYPT;
SEQ ID NO: 108
MQFTLATPKCYGPGA;
SEQ ID NO: 109
MQNPPHSSPHSSPLPRYSYLSPLLY;
SEQ ID NO: 110
MQPYRRYRFHPRLSMIYPTLQLM;
SEQ ID NO: 111
MQRWFFMSLVVVVVRA;
SEQ ID NO: 112
MQSGVLQSLAVTEIKYCNLL;
SEQ ID NO: 113
MQTKSLQSPQ;
SEQ ID NO: 114
MQWAILGLFVVG;
SEQ ID NO: 115
MRLLAWGNTWWQLLWNEGLFFWLELE;
SEQ ID NO: 116
MSGVRKEVSLL;
SEQ ID NO: 117
MSKKIPATTMVLEWS;
SEQ ID NO: 118
MSLSYLQDSTY;
SEQ ID NO: 119
MSLTPHILPTMP;
SEQ ID NO: 120
MSMPVLGWQWG;
SEQ ID NO: 121
MSNMKTFSSA;
SEQ ID NO: 122
MSNNMAFSTFKG;
SEQ ID NO: 123
MSRNHSFCLNYMPIRFSLILFVVTH;
SEQ ID NO: 124
MSSAWIIWFRLFSI;
SEQ ID NO: 125
MSSDEFANTMPV;
SEQ ID NO: 126
MSTHLPNYHTNLSYR;
SEQ ID NO: 127
MSTINQQVIITLTVNPTQACS;
SEQ ID NO: 128
MSWSNIEVVNPIVDMDS;
SEQ ID NO: 129
MSYRAHTPKMLVMPFPY;
SEQ ID NO: 130
MTAPVKLQGVWM;
SEQ ID NO: 131
MTELLTPNWTNLSPY;
SEQ ID NO: 132
MTKNFHQTPPPPLLATALKHISAKPQKQ;
SEQ ID NO: 133
MTLGHNMIYLHTS;
SEQ ID NO: 134
MTNIFGVWAR;
SEQ ID NO: 135
MTNPENQNERKSVRFIHCPHNP;
SEQ ID NO: 136
MTNTLNSIHPPLP;
SEQ ID NO: 137
MTSLHNSFYSKDTSLRTPLMTP;
SEQ ID NO: 138
MTSPKIQNNNTPDHTANNQY;
SEQ ID NO: 139
MTWPTEQVTLGMTAQSYS;
SEQ ID NO: 140
MTYSPEQSQLQYMHQQTMFNQ;
SEQ ID NO: 141
MVEGWLLYLLVSMG;
SEQ ID NO: 142
MVEVGYNVVRVV;
SEQ ID NO: 143
MVGYLIPVWVLAIVCSDMLKPLS;
SEQ ID NO: 144
MVIICFEVSLISHCWVVISRLLM;
SEQ ID NO: 145
MVKPHLQNASI;
SEQ ID NO: 146
MVLEFGLVGYFLLGGGSGWV;
SEQ ID NO: 147
MVLFLLGMVG;
SEQ ID NO: 148
MVLNLRVHRLRRTNLQLLHTSPIIP;
SEQ ID NO: 149
MVLSSPPIFRMSCSLL;
SEQ ID NO: 150
MVLWWFGGSG;
SEQ ID NO: 151
MVNGRRTSVGV;
SEQ ID NO: 152
MVRMLLVQWW;
SEQ ID NO: 153
MVSMMTSMGM;
SEQ ID NO: 154
MVSSGDWYSWCE;
SEQ ID NO: 155
MVVCMVITFIWSCTKIFGA;
SEQ ID NO: 156
MVVHWMSGVGLPWLWGVGV;
SEQ ID NO: 157
MVVKGPLSEGGHPWGREGIWL;
SEQ ID NO: 158
MWEEAGRMSEGCLG;
SEQ ID NO: 159
MWGVSDMLVFLEV;
SEQ ID NO: 160
MWLGVGLGWVG;
SEQ ID NO: 161
MWLIEEYAMSDF;
SEQ ID NO: 162
MWLVLWLWML;
SEQ ID NO: 163
MWMPPTLPHIR;
SEQ ID NO: 164
MWNDLLQCSEP;
SEQ ID NO: 165
MWRVLGALWW;
SEQ ID NO: 166
MWSHPSHHSTINITNKWLL;
SEQ ID NO: 167
MWVIEEYAKILRSWVWFNPPQLPAMMDKIE;
SEQ ID NO: 168
MYCNWDCSGE;
SEQ ID NO: 169
MYLNGTCSAS;
SEQ ID NO: 170
MYNYAKAPTL;
SEQ ID NO: 171
MYRHLQQTLMKATK;
SEQ ID NO: 172
MYTNKQCSTSNYY;
SEQ ID NO: 173
MYTPTNNVQPVTTTNQRP;
SEQ ID NO: 174
MYYSDGYWGVSWGMGV;
SEQ ID NO: 175
MYEMHSGCWWVSQYLGGTQICFPMKEQ;
SEQ ID NO: 176
MKNIEAPLAW;
SEQ ID NO: 177
MRMGASIGSTTRLSTS;
SEQ ID NO: 178
MVQTVSISWASEMLVLVSFVVSV;
SEQ ID NO: 179
MSSSMMGEVASC;
SEQ ID NO: 180
MVVGSWCWSLSWVVGMR;
SEQ ID NO: 181
MCHWAGGASNTGDA;
SEQ ID NO: 182
MFAVSMMWSLE;
SEQ ID NO: 183
MMKVDATMDFT;
SEQ ID NO: 184
MASAKGCSSP;
and
SEQ ID NO: 185
MLVMLEVMFLVN.

6A. A peptide of Paragraph 5A wherein the peptide comprises substitution with at least one amino acid selected from (i) an amino acid having a D-configuration, and (ii) a non-naturally occurring amino acid residue; or pharmaceutically acceptable salts thereof.

7A. A composition comprising a peptide of any one of paragraphs 1A-6A and a pharmaceutically acceptable excipient.

8A. A method of modulating cell viability comprising administering a peptide of any one of paragraphs 1A-6A.

9A. A method of treating cancer in patient in need of such treatment, comprising administering to the patient a pharmacologically effective amount of a peptide of any one of paragraphs 1A-6A.

10A. A method of treating cell proliferation in patient in need of such treatment, comprising administering the patient a pharmacologically effective amount of a peptide of any one of paragraphs 1A-6A.

11A. A method of treating an apoptosic disease in a patient in need of such treatment, comprising administering to the patient a pharmacologically effect amount of a peptide of any one of paragraphs 1A-6A.

12A. A method of treating a metabolic disease in a patient in need of such treatment, comprising administering to the patient a pharmacologically effect amount of a peptide of any one of paragraphs 1A-6A.

13A. A method of providing cytoprotection in a patient in need of such treatment, comprising administering to the patient a pharmacologically effect amount of a peptide of any one of paragraphs 1A-6A.

14A. A peptide of any one of paragraphs 1A-6A, further comprising a duration enhancing moiety coupled to the peptide, and optionally further comprising a metabolically cleavable linker coupling the peptide to the duration enhancing moiety.

15A. The composition of paragraph 7A, wherein the pharmaceutically acceptable excipient is not found in nature.

16A. A pharmaceutical composition comprising a peptide of any of paragraphs 1A-6A.

17A. An isolated nucleic acid that comprises a nucleotide sequence that encodes a peptide of any one of paragraphs 1A-6A.

18A. A vector or expression vector that comprises an isolated nucleic acid according to paragraph 17A.

19A. A host cell that comprises a nucleic acid according to paragraph 17A or a vector or expression vector according to paragraph 18A.

20A. A method of treating a metabolic disease in a subject in need thereof, comprising administering to the subject a peptide or peptide analog, a conjugate, a nucleic acid, an expression vector, a host cell, or a composition of any one of paragraphs 1A-7A and 14A-19A, in an amount effective to treat the metabolic disease.

21A. The method of paragraph 20A, wherein the disease is selected from the group consisting of obesity, diabetes (e.g., Type 2 diabetes), cognitive disorders and/or neurodegenerative disorders, cardiovascular disease, fatty liver disease, and gastrointestinal disease.

22A. A method of treating a cancer in a subject in need thereof, comprising administering to the subject a peptide or peptide analog, a conjugate, a nucleic acid, an expression vector, a host cell, or a composition of any one of paragraphs 1A-7A and 14A-19A, in an amount effective to treat the cancer.

23A. The method of paragraph 22A, wherein the cancer is lung cancer, pancreatic cancer, breast cancer, prostate cancer, ovarian cancer, or hepatocellular cancer.

24A. A method of treating a liver disease in a subject in need thereof, comprising administering to the subject a peptide or peptide analog, a conjugate, a nucleic acid, an expression vector, a host cell, or a composition of any one of paragraphs 1A-7A and 14A-19A, in an amount effective to treat the liver disease.

25A. The method of paragraph 24A, wherein the liver disease is a fatty liver disease.

26A. The method of paragraph 25A, wherein the fatty liver disease is NAFLD or NASH.

27A. A method of modulating fatty acid metabolism in a subject in need thereof, comprising administering to the subject a peptide or peptide analog, a conjugate, a nucleic acid, an expression vector, a host cell, or a composition of any one of paragraphs 1A-7A and 14A-19A, in an amount effective to modulate fatty acid metabolism.

28A. The method of paragraph 27A, wherein fatty acid metabolism is increased in the subject after the peptide or peptide analog, conjugate, nucleic acid, expression vector, or host cell is administered to the subject.

29A. A method of reducing body weight in a subject in need thereof, comprising administering to the subject a peptide or peptide analog, a conjugate, a nucleic acid, an expression vector, a host cell, or a composition of any one of paragraphs 1A-7A and 14A-19A, in an amount effective to reduce body weight in the subject.

30A. Use of any one of the peptides, peptide analogs, conjugates, nucleic acids, expression vectors, host cells, or compositions of any one of paragraphs 1A-7A and 14A-19A, in therapeutic treatment of a metabolic disease, cancer, liver disease, or any disease, disorder, or medical condition described herein.

31A. Use of any one of the peptides, peptide analogs, conjugates, nucleic acids, expression vectors, host cells, or compositions of any one of paragraphs 1A-7A and 14A-19A, in the manufacture of a medicament for treating a metabolic disease, cancer, liver disease, or any disease, disorder, or medical condition described herein.

The peptides and their uses having been described, the following examples are offered by way of illustration, and not limitation.

### EXAMPLES

### Example 1

The peptides are prepared via solid phase synthesis on a suitable resin using t-Boc or Fmoc chemistry or other well established techniques, (see for example: Stewart and Young, Solid Phase Peptide Synthesis, Pierce Chemical Co., Rockford, III., 1984; E. Atherton and R. C. Sheppard, Solid Phase Peptide Synthesis. A Practical Approach, Oxford-IRL Press, New York, 1989; Greene and Wuts, “Protective Groups in Organic Synthesis”, John Wiley & Sons, 1999, Florencio Zaragoza Dorwald, “Organic Synthesis on solid Phase”, Wiley-VCH Verlag GmbH, 2000, and “Fmoc Solid Phase Peptide Synthesis”, Edited by W. C. Chan and P. D. White, Oxford University Press, 2000) by a method similar to that described below, unless specified otherwise.

Solid phase synthesis is initiated by attaching an N-terminally protected amino acid with its carboxy terminus to an inert solid support carrying a cleavable linker. This solid support can be any polymer that allows coupling of the initial amino acid, e.g. a Pam resin, trityl resin, a chlorotrityl resin, a Wang resin or a Rink resin in which the linkage of the carboxy group (or carboxamide for Rink resin) to the resin is sensitive to acid (when Fmoc strategy is used). The polymer support is stable under the conditions used to deprotect the α-amino group during the peptide synthesis. After the first amino acid has been coupled to the solid support, the α-amino protecting group of this amino acid is removed. The remaining protected amino acids are then coupled one after the other in the order represented by the peptide sequence using appropriate amide coupling reagents, for example BOP (benzotriazol-1-yl-oxy-tris-(dimethylamino)-phosphonium), HBTU (2-(1H-benzotriazol-1-yl)-1,1,3,3-tetramethyl-uronium), HATU (O-(7-azabenztriazol-1-yl-oxy-tris-(dimethylamino)-phosphonium) or DIC (N,N′-diisopropylcarbodiimide)/HOBt (1-hydroxybenzotriazol), wherein BOP, HBTU and HATU are used with tertiary amine bases. Alternatively, the liberated N-terminus can be functionalized with groups other than amino acids, for example carboxylic acids, etc. Usually, reactive side-chain groups of the amino acids are protected with suitable blocking groups. These protecting groups are removed after the desired peptides have been assembled. They are removed concomitantly with the cleavage of the desired product from the resin under the same conditions. Protecting groups and the procedures to introduce protecting groups can be found in Protective Groups in Organic Synthesis, 3d ed., Greene, T. W. and Wuts, P. G. M., Wiley & Sons (New York: 1999). In some cases, it might be desirable to have side-chain protecting groups that can selectively be removed while other side-chain protecting groups remain intact. In this case the liberated functionality can be selectively functionalized. For example, a lysine may be protected with an ivDde protecting group (S. R. Chhabra et al., Tetrahedron Lett. 39, (1998), 1603) which is labile to a very nucleophilic base, for example 4% hydrazine in DMF (dimethyl formamide). Thus, if the N-terminal amino group and all side-chain functionalities are protected with acid labile protecting groups, the ivDde ([1-(4,4-dimethyl-2,6-dioxocyclohex-1-ylidene)-3-methylbutyl) group can be selectively removed using 4% hydrazine in DMF and the corresponding free amino group can then be further modified, e.g. by acylation. The lysine can alternatively be coupled to a protected amino acid and the amino group of this amino acid can then be deprotected resulting in another free amino group which can be acylated or attached to further amino acids. Finally, the peptide is cleaved from the resin. This can be achieved by using HF or King's cocktail (D. S. King, C. G. Fields, G. B. Fields, Int. J. Peptide Protein Res. 36, 1990, 255-266). The raw material can then be purified by chromatography, e.g. preparative RP-HPLC, if necessary.

Those peptides, analogs or derivatives which include non-natural amino acids and/or a covalently attached N-terminal mono- or dipeptide mimetic may be produced as described in the experimental part. Or see e.g., Hodgson et al: “The synthesis of peptides and proteins containing non-natural amino acids”, and Chemical Society Reviews, vol. 33, no. 7 (2004), p. 422-430.

The peptides are prepared according to the below-mentioned peptide synthesis and the sequences as presented in the Table 1 can be prepared similar to the below-mentioned synthesis, unless specified otherwise.

One method of peptide synthesis is by Fmoc chemistry on a microwave-based Liberty peptide synthesizer (CEM Corp., North Carolina). The resin is Tentagel S RAM with a loading of about 0.25 mmol/g or PAL-ChemMatrix with a loading of about 0.43 mmol/g or PAL AM matrix with a loading of 0.5-0.75 mmol/g. The coupling chemistry is DIC/HOAt or DIC/Oxyma in NMP or DMF using amino acid solutions of 0.3 M and a molar excess of 6-8 fold. Coupling conditions are 5 minutes at up to 70° C. Deprotection is with 10% piperidine in NMP at up to 70° C. The protected amino acids used are standard Fmoc-amino acids (supplied from e.g. Anaspec or Novabiochem or Protein Technologies).

Another method of peptide synthesis is by Fmoc chemistry on a Prelude peptide synthesizer (Protein Technologies, Arizona). The resin is Tentagel S RAM with a loading of about 0.25 mmol/g or PAL-ChemMatrix with a loading of about 0.43 mmol/g or PAL AM with a loading of 0.5-0.75 mmol/g. The coupling chemistry is DIC/HOAt or DIC/Oxyma in NMP or DMF using amino acid solutions of 0.3 M and a molar excess of 6-8 fold. Coupling conditions are single or double couplings for 1 or 2 hours at room temperature. Deprotection is with 20% piperidine in NMP. The protected amino acids used are standard Fmoc-amino acids (supplied from e.g. Anaspec or Novabiochem or Protein Technologies). The crude peptides are purified such as by semipreparative HPLC on a 20 mm×250 mm column packed with either 5 um or 7 um C-18 silica. Peptide solutions are pumped onto the HPLC column and precipitated peptides are dissolved in 5 ml 50% acetic acid H₂O and diluted to 20 ml with H₂O and injected on the column which then is eluted with a gradient of 40-60% CH₃CN in 0.1% TFA 10 ml/min during 50 min at 40° C. The peptide containing fractions are collected. The purified peptide is lyophilized after dilution of the eluate with water.

All peptides with C terminal amides described herein are prepared by a method similar to that described below unless specified otherwise. MBHA resin (4-methylbenzhydrylamine polystyrene resin is used during peptide synthesis. MBHA resin, 100-180 mesh, 1% DVB cross-linked polystyrene; loading of 0.7-1.0 mmol/g), Boc-protected and Fmoc protected amino acids can be purchased from Midwest Biotech. The solid phase peptide syntheses using Boc-protected amino acids are performed on an Applied Biosystem 430A Peptide Synthesizer. Fmoc protected amino acid synthesis is performed using the Applied Biosystems Model 433 Peptide Synthesizer.

Synthesis of the peptides is performed on the Applied Biosystem Model 430A Peptide Synthesizer. Synthetic peptides are constructed by sequential addition of amino acids to a cartridge containing 2 mmol of Boc protected amino acid. Specifically, the synthesis is carried out using Boc DEPBT-activated single couplings. At the end of the coupling step, the peptidyl-resin is treated with TFA to remove the N-terminal Boc protecting group. It is washed repeatedly with DMF and this repetitive cycle is repeated for the desired number of coupling steps. After the assembly, the sidechain protection, Fmoc, is removed by 20% piperidine treatment and acylation was conducted using DIC. The peptidyl-resin at the end of the entire synthesis is dried by using DCM, and the peptide is cleaved from the resin with anhydrous HF. The peptidyl-resin is treated with anhydrous HF, and this typically yielded approximately 350 mg (˜50% yield) of a crude deprotected-peptide. Specifically, the peptidyl-resin (30 mg to 200 mg) is placed in the hydrogen fluoride (HF) reaction vessel for cleavage. 500 μL of p-cresol was added to the vessel as a carbonium ion scavenger. The vessel is attached to the HF system and submerged in the methanol/dry ice mixture. The vessel is evacuated with a vacuum pump and 10 ml of HF is distilled to the reaction vessel. This reaction mixture of the peptidyl-resin and the HF is stirred for one hour at 0° C., after which a vacuum is established and the HF is quickly evacuated (10-15 min). The vessel is removed carefully and filled with approximately 35 ml of ether to precipitate the peptide and to extract the p-cresol and small molecule organic protecting groups resulting from HF treatment. This mixture is filtered utilizing a Teflon filter and repeated twice to remove all excess cresol. This filtrate is discarded. The precipitated peptide dissolves in approximately 20 ml of 10% acetic acid (aq). This filtrate, which contained the desired peptide, is collected and lyophilized.

### Example 2.—Caspase 37 Activity

The effect of the peptides on cell death/survival can be assessed using a caspase-3/7 assay in cultured cells. Peptides are dissolved at 10 mM in DMSO as stock solutions for use at a final concentration of 10 μM. Staurosporine is used as a highly potent positive control for caspase induction. Staurosporine (Selleckchem) is dissolved at 1 mM in DMSO as a stock solution. Caspase-Glo 3/7 Assay Reagent is purchased from Promega (Madison, Wis.). A172 human brain glioblastoma cell line is purchased from American Type Culture Collection (Manassas, Va.). A172 cells are grown in DMEM supplemented with 10% FBS with 100 IU/ml penicillin and 100 μg/ml streptomycin. Cultures are maintained at 37° C. in a humidified atmosphere of 5% CO2/95% air. A172 cells are seeded at 8,000 cells per well on 96-well plates. The next day cells are incubated with test peptides at 10 μM or staurosporine at concentrations between 10 nM and 1 μM using a final concentration of 0.1% DMSO and maintained at 37° C. in a humidified atmosphere of 5% CO2/95% air for 18-20 hours. Caspase 3/7 activity is determined using a Caspase-Glo 3/7 Assay kit (Promega) according to the manufacturer's instructions. Luminescence for each sample well on the plate is measured using a Cytation 3 plate reader (BioTek, Winooski, Vt.). Activity is calculated relative to 0.1% DMSO control. The relative standard deviation of the DMSO control is <10%. Caspase 3/7 activity of staurosporine (10 nM) treatment is 189% of the background-corrected DMSO control value. Administration of the peptides of the invention alone and/or in combination with positive control results in increased or decreased caspase 3/7 activity in MDA-MB-231 cells than those treated with vehicle control.

### Example 3.—Cell Viability

The effect of the peptides on cell viability can be assessed in cultured cells using suitable assay of viability such as the PrestoBlue® assay (Thermo Fisher Scientific, Waltham, Mass.). Peptides are initially prepared as 10 mM stock in DMSO and tested at a final concentration of 10 μM (0.1% DMSO). Staurosporine is used as a highly potent positive control for induction of apoptosis/cell death. Staurosporine is dissolved with DMSO and tested at final concentrations between 10 nM and 1 μM (0.1% DMSO). PrestoBlue Assay Reagent is purchased from Thermo Fisher Scientific. A172 human brain glioblastoma cell line is purchased from American Type Culture Collection (Manassas, Va.). A172 cells are grown in DMEM supplemented with 10% FBS with 100 IU/ml penicillin and 100 μg/ml streptomycin. Cultures are maintained at 37° C. in a humidified atmosphere of 5% CO₂/95% air. A172 cells are seeded at 8,000 cells per well on 96-well plates. The next day cells are incubated with test peptides at 10 μM or staurosporine using a final concentration of 0.1% DMSO and maintained at 37° C. in a humidified atmosphere of 5% CO₂/95% air for 18-20 hours. Cell viability is determined using a PrestoBlue Assay reagent (Promega) according to the manufacturer's instructions. Absorption for each sample well on the plate is measured using a Cytation 3 plate reader at 560 nm and 590 nm (BioTek, Winooski, Vt.). Activity is calculated relative to 0.1% DMSO untreated control. Treatment with 0.1% DMSO alone is used as the cell viability activity control. Staurosporine is used as a highly potent positive control for decreasing cell viability. Administration of the peptides of the invention alone and/or in combination with positive control results in increased or decreased cell viability in A-172 cells than those treated with vehicle control.

### Example 4.—Free Fatty Acid Levels in Cultured Mouse Adipocytes

The effect of the peptides on fatty acid metabolism can be assessed using an assay of free fatty acid levels in cultured cells such as mouse adipocytes. Peptides are initially prepared as 10 mM stock in DMSO and used at a final concentration of 10 μM (0.1% DMSO). Isoproterenol is used as a highly potent inducer of fatty acid production. Mouse 3T3-L1 cells purchased from ZenBio are seeded at 3,000 cells per well in 96-well plates in Pre-adipocyte Medium (Zen-Bio) and grown to confluence at 37° C. in a humidified atmosphere of 5% CO₂/95% air. Two days after confluence, cells are placed in Adipocyte Differentiation Medium (Zen-Bio) and cultured for three additional days at 37° C. in a humidified atmosphere of 5% CO2/95% air. The culture media is then replaced with Adipocyte Maintenance Medium (Zen-Bio) and the cells maintained for an additional 9-12 days at 37° C. in a humidified atmosphere of 5% CO₂/95% air with partial medium replacement every other day. Following 12-15 days of differentiation, test peptides are added at a final concentration of 10 μM in 0.1% DMSO and incubated for 20-22 hours in Adipocyte Maintenance Medium at 37° C. in a humidified atmosphere of 5% CO₂/95% air. After 20-22 hours, 1 nM isoproterenol is added to all wells except untreated controls and test peptides are replenished. Insulin at 100 nM is added to control wells. Cells are incubated for 3 hours in Assay Buffer (Zen-Bio) at 37° C. in a humidified atmosphere of 5% CO₂/95% air. Free fatty acid concentrations in the media are determined using a Free Fatty Acid Assay kit (Zen-Bio) according to the manufacturer's instructions using a Cytation 3 plate reader at 540 nm (BioTek, Winooski, Vt.). Absorbance values are corrected for untreated background and expressed relative to isoproterenol treated cells. Treatment with isoproterenol (1 nM) alone is used as the free fatty acid level stimulatory control. Insulin is used as a highly potent positive control for decreasing free fatty acid levels. Administration of the peptides of the invention alone and/or in combination with positive control results in increased or decreased free fatty acid levels in cultured 3T3-L1 mouse adipocytes than those treated with vehicle control.

### Example 5.—Glucose Utilization

The effect of the peptides on glucose metabolism can be assessed using an assay of glucose utilization in cultured cultured cells such as mouse myoblasts. Peptides are initially prepared as 10 mM stock in DMSO and used at a final concentration of 10 μM (0.1% DMSO). C2C12 mouse myoblast cell line is purchased from American Type Culture Collection (Manassas, Va.). C2C12 cultures are maintained at 37° C. in a humidified atmosphere of 5% CO2/95% air with medium changes every second day. C2C12 cells are grown in DMEM (1 g/L glucose) supplemented with 10% FBS with 100 IU/ml penicillin and 100 μg/ml streptomycin. C2C12 cells are seeded at 7,000 cells per well on 96-well plates and cultured to confluence. Once the cell reached confluence the media is changed to DMEM (1 g/L glucose) supplemented with 2% HS with 100 IU/ml penicillin and 100 μg/ml streptomycin and maintained at 37° C. in a humidified atmosphere of 5% CO2/95% air. 5 days post-induction of differentiation fresh DMEM (1 g/L glucose) supplemented with 2% HS with 100 IU/ml penicillin and 100 μg/ml streptomycin is added to cultures. Cells are maintained at 37° C. in a humidified atmosphere of 5% CO₂ and 95% air for 5 hours. After 5 hours test peptides at 10 μM or controls (0.5 mM or 1 mM metformin in 1% DMSO) prepared in fresh differentiation medium are added to cells and the cultures are maintained at 37° C. in a humidified atmosphere of 5% CO2/95% air for 18-22 hours. At the end of the incubation culture media is removed from the cells and the remaining glucose concentration is measured using a Glucose Assay kit (Abcam) according to the manufacturer's instructions, using a Cytation 3 plate reader at 570 nm (BioTek, Winooski, Vt.). Glucose concentrations in the medium are calculated relative to 0.1% DMSO treated control cells. Metformin is used as a positive control for reduction of glucose levels. Administration of the peptides of the invention alone and/or in combination with positive control results in increased or decreased glucose utilization in C2C12 mouse myoblasts than those treated with vehicle control.

### Example 6—ATP Levels

The effect of the peptides on cellular metabolism can be assessed using an assay of ATP levels in cultured cells such as human neuroblastoma cells. Peptides are initially prepared as 10 mM stock in DMSO and used at a final concentration of 10 μM (0.1% DMSO). Staurosporine is used as a highly potent positive control for induction of apoptosis/cell death resulting in reduction of ATP levels. Staurosporine is dissolved in DMSO and used at final concentrations between 10 nM and 1 μM in 0.1% DMSO. CellTiter-Glo® Assay kit is purchased from Promega. SH-SY5Y human bone marrow neuroblastoma cell line is purchased from American Type Culture Collection (Manassas, Va.) and licensed from Memorial Sloan-Kettering Cancer Center (New York, N.Y.). SH-SY5Y cells are grown in DMEM/F12 medium supplemented with 10% FBS with 100 IU/ml penicillin and 100 μg/ml streptomycin. Cultures are maintained at 37° C. in a humidified atmosphere of 5% CO2/95% air. SH-SY5Y cells are seeded at 30,000 cells per well on 96-well plates. The next day cells are incubated with test peptides at 10 μM or staurosporine at the indicated concentration in 0.1% DMSO and maintained at 37° C. in a humidified atmosphere of 5% CO2/95% air for 18-20 hours. ATP levels are determined using a CellTiter-Glo Assay kit (Promega) according to the manufacturer's instructions. Luminescence for each sample well on the plate is measured using a Cytation 3 plate reader (BioTek, Winooski, Vt.). Activity is calculated relative to 0.1% DMSO treated control. Staurosporine is used as a highly potent positive control for reduction of ATP levels. Administration of the peptides of the invention alone and/or in combination with positive control results in increased or decreased ATP levels in cultured SH-SY5Y nuroblastoma cells than those treated with vehicle control.

### Example 7—ATP Levels in Cells Exposed to Staurosporine

The potential cytoprotective effects or potential synergistic effects on cell viability of the peptides can be assessed using an assay of ATP levels in cultured cells such as human neuroblastoma cells exposed to a suitable stress such as staurosporine exposure. Peptides are initially prepared as 10 mM stock in DMSO and tested at a final concentration of 10 μM (0.1% DMSO). Staurosporine is used as a highly potent inducer of apoptosis/cell death that reduces cellular ATP levels. Staurosporin is used at concentrations ranging from 10 nM to 1 μM. CellTiter-Glo® Assay kit is purchased from Promega. SH-SY5Y human neuroblastoma cell line is purchased from American Type Culture Collection (Manassas, Va.) and licensed from Memorial Sloan-Kettering Cancer Center (New York, N.Y.). SH-SY5Y cells are grown in DMEM/F12 medium supplemented with 10% FBS with 100 IU/ml penicillin and 100 μg/ml streptomycin. Cultures are maintained at 37° C. in a humidified atmosphere of 5% CO2/95% air. SH-SY5Y cells are seeded at 30,000 cells per well on 96-well plates. The next day cells are incubated with test peptides at 10 μM in 0.1% DMSO and staurosporine (40 μM) and maintained at 37° C. in a humidified atmosphere of 5% CO2/95% air for 18-20 hours. ATP levels are determined using a CellTiter-Glo Assay kit (Promega) according to the manufacturer's instructions. Luminescence for each sample well on the plate is measured using a Cytation 3 plate reader (BioTek, Winooski, Vt.). Activity is calculated relative to the reduction in ATP by treatment with 40 μM staurosporine. A value less than 100% is indicative of a cytoprotective effect, while a value greater than 100% is indicative of a synergistic effect on viability. The relative standard deviation of the result for the 40 μM staurosporine treated control cells is <5%. Administration of the peptides of the invention alone and/or in combination with positive control results in increased or decreased ATP levels in cultured SH-SY5Y nuroblastoma cells exposed to staurosporine than those treated with vehicle control.

### Example 8—Cell Proliferation

The effect of the peptides on cell proliferation is assessed using a DNA dye binding assay in cultured cells. Peptides are initially prepared as 10 mM stock in DMSO and diluted to 1 mM in H2O and added at a final concentration of 10 μM (0.1% DMSO). Lovastatin is used as a growth arrest control at final concentration of 10 μM. C2C12 mouse muscle myoblast cell line is purchased from American Type Culture Collection (Manassas, Va.). C2C12 cells are grown in DMEM supplemented with 10% FBS with 100 IU/ml penicillin and 100 μg/ml streptomycin. Cultures are maintained at 37° C. in a humidified atmosphere of 5% CO2/95% air. C2C12 cells are seeded at 2,000 cells per well on 96-well plates. The next day cells are incubated with test peptides at 10 μM in 0.1% DMSO and maintained at 37° C. in a humidified atmosphere of 5% CO2/95% air for 48 hours with readdition of compounds at 24 hour. Cell proliferation is determined using Cyquant Direct Nucleic Acid Stain (Thermo Fisher Scientific, Waltham, Mass.) according to the manufacturer's instructions. Fluorescence for each sample well on the plate is measured using a Cytation 3 plate reader at at Ex/Em=495/535 nm (BioTek, Winooski, Vt.). Activity is calculated relative to 0.1% DMSO untreated control. Administration of the peptides of the invention alone and/or in combination with positive control results in increased or decreased cell proliferation in C2C12 cells than those treated with vehicle control.

### Example 9—Levels of Reactive Oxygen Species

The protective or synergistic effect of the peptides on cellular levels of reactive oxygen species (ROS) induced by oxidative stress can be assessed using an assay of ROS in cultured cells exposed to a suitable oxidative stress. Peptides are initially prepared as 10 mM stock in DMSO and diluted to 1 mM in H2O and added at a final concentration of 10 μM (0.1% DMSO). Tert-butyl hydrogen peroxide (TBHP) is used as a highly potent inducer of ROS. TBHP is used at final concentration of 100 μM. Glutathione ethyl ester (GEE) at final concentration of 5 mM or sulforaphane at final concentration of 10 uM are used as protective controls against TBHP induced ROS production. C2C12 mouse muscle myoblast cell line is purchased from American Type Culture Collection (Manassas, Va.). C2C12 cells are grown in DMEM supplemented with 10% FBS with 100 IU/ml penicillin and 100 μg/ml streptomycin. Cultures are maintained at 37° C. in a humidified atmosphere of 5% CO2/95% air. C2C12 cells are seeded at 7,500 cells per well on 96-well plates. Two days after seeding cells are incubated with test peptides at 10 μM in 0.1% DMSO or sulforaphane at 10 uM and maintained at 37° C. in a humidified atmosphere of 5% CO2/95% air for 18-20 hours. After 18-20 hours of incubation the cells are loaded with DCFDA for 45 min. TBHP at 100 μM and GEE at 5 mM are then added to the appropriate wells for 1 hour. ROS activity is determined using a DCFDA Cellular ROS Detection Assay kit (Abcam, Cambridge, Mass.) according to the manufacturer's instructions. Fluorescence in each sample well on the plate is measured using a Cytation 3 plate reader at Ex/Em=485/535 nm (BioTek, Winooski, Vt.). Activity is calculated relative to TBHP control. Administration of the peptides of the invention alone and/or in combination with positive control results in increased or decreased cellular ROS levels induced by TBHP in C2C12 cells than those treated with vehicle control.

### Example 10—Effects on Metabolic Parameters in Diet Induced Obese (DIO) Mice

DIO mouse studies are conducted by methods well known in the art. C57BL/6 mice are maintained on a high fat diet for 6 to 48 weeks to develop diet induced obesity. Animals are randomized to treatment groups based on blood glucose levels and/or body weight. The peptides of the invention or vehicle control are administered daily or twice daily by intraperitoneal or subcutaneous injection for 5 to 21 days. Body weight, blood glucose levels and food intake are monitored. Glucose tolerance is assessed by intraperitoneal administration of glucose (1 to 3 g/kg) followed by measurement of blood glucose levels over 2 hours. Administration of the peptides of the invention results in one or more effects selected from greater body weight loss, greater reduction in blood glucose, and improved glucose tolerance, when compared to animals treated with vehicle control.

### Example 11—Mouse Xenograft Models

Mouse xenograft models are prepared by methods well known in the art. For example, SCID mice are injected with human tumor cells (for example, MCF-7, MDA-MB-231, PC-3, or the like) and tumor growth is monitored. When tumors are of sufficient size, animals are randomized to treatment groups and dosed daily, every other day, or weekly with the peptides of the invention, vehicle control, positive control (e.g., gemcitabine or paclitaxel) or the combination of the peptides of the invention+positive control. Tumor growth, body weight, and survival are monitored over 14 to 28 days. Administration of the peptides of the invention alone and/or in combination with positive control results in decreased tumor growth and/or extension of survival when compared to animals treated with vehicle control.

### Example 12—Protection of Cells from Cytotoxic Insults

Cells (for example primary cultures of rodent cerebral cells, rodent or human nerve-derived cell lines, and the like) are cultured by methods well known in the art. Cells are treated with the peptides of the invention, vehicle control, or positive controls and cells are exposed to a cytotoxic condition, for example, addition of glutamic acid, removal of serum, generation of reactive oxygen species, addition of beta-amyloid protein, exposure to a cytotoxic agent (e.g., MPTP, staurosporine, oligomycin, etc.), exposure to a chemotherapeutic agent (e.g., cisplatin, etc.), and the like. Cell survival is measured by methods well known in the art (for example measurement of lactate dehydrogenase (LDH) activity in cell extracts; measurement of intracellular ATP, MTT (3-(4,5-dimethyl-2-thiazolyl)-2,5-diphenyl-2H-tetrazolium bromide) assay; MTS (3 (4,5-dimethylthiazol-2-yl)-5-(3-carboxymethoxyphenyl)-2 (4-sulfophenyl)-2H-tetrazolium) assay; trypan blue staining; calcein staining; etc.). Treatment of cells with the peptides of the invention prior to and/or during exposure to the cytotoxic condition produces an increase in cell survival when compared to cells treated with vehicle control.

### Example 13—Stability in Plasma

The metabolic stability of the peptides can be assessed in vitro by incubation in plasma. Peptides (100 uM) are incubated in pooled plasma from a suitable species such as rodent or primate species at 37° C. and samples are removed and immediately analyzed for the concentration of intact peptide by LC/MS/MS over the course of 3 hours. The percent of peptide remaining in plasma at each time point is calculated relative to the initial peak area.

### Example 14—Effects on Triglyceride Levels and Markers of Liver Damage

The effects of the peptides on circulating levels of triglycerides and markers of liver damage can be assessed in suitable animal models. Male C57BU/6 mice are maintained on a high fat diet for 12 to 22 weeks to develop diet induced obesity. Animals are randomized to treatment groups based on blood glucose levels and body weight. The peptides are administered to groups of male DIO mice once or twice daily by appropriate routes for between 5 and 28 days. Additional groups of male DIO mice (n=8/group) receive control test articles or vehicle alone. Serum samples are obtained at termination. Samples are analyzed for standard clinical chemistry parameters by methods well known in the art. Seum concentrations of truglycerides and markers of liver damage such as ALT and AST are compared to those in animals treated with vehicle alone.

### Example 15—Pharmacokinetics in Cynomolgus Monkeys

Male cynomolgus monkeys (2 to 6 kg) are fasted for 8 hours prior to dosing. Groups of animals are injected with a single dose of the test peptide (0.1 to 15 mg/kg) by a suitable route. Blood samples are withdrawn at intervals over 24 hours and processed for plasma. Food is returned at four hours post-injection. Concentrations of peptides and/or metabolites in plasma samples are determined by suitable analytical methods (e.g., LC/MS-MS) and pharmacokinetic parameters are calculated by non-compartmental methods.

### Example 16—Effects in a Non-Human Primate Model of Obesity

Spontaneously obese male cynomolgus monkeys are acclimated to dosing and handling for at least 3 weeks. Baseline animal characteristics are determined and animals are randomized into treatment groups based upon body weight and baseline metabolic parameters such as triglyceride levels. Following randomization, groups of monkeys receive daily or twice daily doses of the peptides of the present invention administered by a suitable route for 4 or more weeks. Control groups of monkeys receive daily doses of vehicle or positive control. Food consumption and body weight are measured at intervals during the study. Effects of the administered peptides on body weight, food intake, BMI and/or metabolic parameters are compared to control animals treated with vehicle.

### Example 17

### Effects in the STAM® Mouse Model of Non-alcoholic Steatohepatitis (NASH)

In the STAM model of NASH, C57/bl6 mice are injected with a single subcutaneous dose of streptotoxin, three days after birth to destroy pancreatic cells. At the age of 4 weeks, animals are put on a high fat diet. This combined treatment results in the development of steatosis, fibrosis, cirrhosis and finally hepatocellularcarcinoma (HCC) along with hyperglycemia and moderate hyperlipidemia thus closely resembling human NASH. Beginning at 5 weeks of age, groups of STAM animals (8 animals per group) are treated with the peptides of the present invention administered daily or twice daily by an appropriate route, until study termination. A control group of animals receive daily administration of a suitable positive control compound (e.g. telmisartan). At approximately 10 weeks of age, metabolic parameters are determined and animals are sacrificed. Liver samples are obtained and fixed, embedded in paraffin, stained with hematoxylin and eosin or Masson's trichrome, and examined by light microscopy. The extent of steatosis and the non-alcoholic fatty liver disease (NAFLD) activity score (NAS) are determined histopathologically according to methods known in the art.

### Example 18—Free Fatty Acid Levels in Cultured Mouse Adipocytes

The effect of the peptides on fatty acid metabolism can be assessed using an assay to monitor free fatty acid levels produced by cultured cells such as mouse adipocytes. Peptide arrays were initially prepared as 5 mM stock in DMSO and diluted to 0.5 mM in H₂O; peptides were used at a final concentration of 5 μM (0.1% DMSO). Calculations for peptide concentrations based on approximate molecular weight of 2000 Daltons. Isoproterenol was used as a highly potent inducer of free fatty acid production. Mouse 3T3-L1 cells purchased from ZenBio (Research Triangle Park, NC) were seeded at 3,000 cells per well in 96-well plates in Pre-adipocyte Medium (Zen-Bio) and grown to confluence at 37° C. in a humidified atmosphere of 5% CO2/95% air. Two days after confluence, cells were placed in Adipocyte Differentiation Medium (Zen-Bio) and cultured for three additional days at 37° C. in a humidified atmosphere of 5% CO2/95% air. The culture medium was then replaced with Adipocyte Maintenance Medium (Zen-Bio) and the cells maintained for an additional 9 days at 37° C. in a humidified atmosphere of 5% CO2/95% air with partial medium replacement every other day. Following 12 days of differentiation, medium was replaced Adipocyte Maintenance Medium without insulin and the cultures placed at 37° C. in a humidified atmosphere of 5% CO₂/95% air. On day 13 of differentiation, test peptides were added directly to each well (without replacing the medium) at a final concentration of 5 μM in 0.1% DMSO and incubated for 20-22 hours at 37° C. in a humidified atmosphere of 5% CO2/95% air. After 20-22 hours, the medium was removed and replaced with Assay Buffer (ZenBio) containing the appropriate compounds; 0.1 nM isoproterenol was added to all wells except untreated controls, test peptides were reapplied to appropriate wells at 5 μM, and 0.25 nM insulin was added to control wells as a highly potent inhibitor of free fatty acid production. Cells were incubated for 3 hours in Assay Buffer (Zen-Bio) at 37° C. in a humidified atmosphere of 5% CO2/95% air. Following 3 h incubation, conditioned medium from each well was transferred to a fresh 96-well plate. Free fatty acid concentrations in the medium were determined using a Free Fatty Acid Assay kit (Zen-Bio) according to the manufacturer's instructions; absorbance was measured using a Cytation 3 plate reader at 540 nm (BioTek, Winooski, Vt.). Treatment with 0.1 nM isoproterenol alone was used as the free fatty acid level stimulatory control. Absorbance values were expressed as percent control activity relative to 0.1 nM isoproterenol alone treated cells, 100%. The relative standard deviation of the isoproterenol control was <10%. Insulin at 0.25 nM was used as a potent control for decreasing free fatty acid levels. Free fatty acid levels for insulin (0.25 nM) treatment were at or below levels for unstimulated control without isoproterenol. Data presented are representative values from 2 independent experiments with each data point performed in triplicate in each independent experiment. The results are reported in Table 4.

TABLE 4
Free Fatty Acid Levels in Cultured
3T3-L1 Mouse Adipocytes

| | Percent of |
|---|---|
| | Isoproterenol |
| SEQ ID NO: | Control Activity |
| 1 | 85 |
| 2 | 93 |
| 3 | 91 |
| 4 | 80 |
| 5 | 98 |
| 6 | 80 |
| 8 | 102 |
| 9 | 98 |
| 10 | 94 |
| 11 | 84 |
| 13 | 92 |
| 14 | 101 |
| 15 | 90 |
| 16 | 89 |
| 18 | 102 |
| 19 | 89 |
| 20 | 88 |
| 23 | 94 |
| 24 | 92 |
| 25 | 91 |
| 26 | 93 |
| 27 | 87 |
| 28 | 105 |
| 29 | 90 |
| 30 | 98 |
| 31 | 78 |
| 32 | 94 |
| 33 | 103 |
| 35 | 98 |
| 36 | 103 |
| 37 | 106 |
| 38 | 103 |
| 39 | 99 |
| 40 | 95 |
| 41 | 99 |
| 42 | 106 |
| 43 | 103 |
| 44 | 112 |
| 45 | 98 |
| 46 | 98 |
| 47 | 96 |
| 48 | 91 |
| 49 | 87 |
| 50 | 100 |
| 51 | 89 |
| 52 | 92 |
| 53 | 91 |
| 54 | 104 |
| 55 | 98 |
| 56 | 89 |
| 57 | 102 |
| 58 | 105 |
| 59 | 100 |
| 60 | 94 |
| 61 | 86 |
| 62 | 94 |
| 65 | 99 |
| 66 | 104 |
| 67 | 116 |
| 68 | 99 |
| 69 | 104 |
| 70 | 110 |
| 71 | 89 |
| 72 | 96 |
| 73 | 98 |
| 75 | 79 |
| 78 | 96 |
| 79 | 90 |
| 80 | 87 |
| 81 | 90 |
| 82 | 98 |
| 83 | 102 |
| 84 | 99 |
| 85 | 81 |
| 86 | 98 |
| 87 | 101 |
| 88 | 95 |
| 89 | 111 |
| 90 | 101 |
| 91 | 93 |
| 92 | 99 |
| 93 | 96 |
| 95 | 111 |
| 96 | 100 |
| 97 | 105 |
| 98 | 97 |
| 100 | 105 |
| 102 | 120 |
| 103 | 108 |
| 105 | 91 |
| 106 | 98 |
| 107 | 104 |
| 108 | 102 |
| 109 | 94 |
| 110 | 93 |
| 111 | 89 |
| 112 | 103 |
| 113 | 102 |
| 114 | 76 |
| 116 | 90 |
| 117 | 98 |
| 118 | 98 |
| 119 | 97 |
| 120 | 95 |
| 121 | 118 |
| 122 | 113 |
| 123 | 104 |
| 124 | 113 |
| 125 | 115 |
| 126 | 108 |
| 127 | 104 |
| 128 | 116 |
| 129 | 115 |
| 130 | 110 |
| 131 | 110 |
| 133 | 107 |
| 134 | 111 |
| 135 | 127 |
| 136 | 124 |
| 137 | 115 |
| 138 | 111 |
| 139 | 91 |
| 140 | 100 |
| 141 | 96 |
| 142 | 97 |
| 143 | 107 |
| 144 | 96 |
| 145 | 94 |
| 146 | 97 |
| 147 | 99 |
| 148 | 103 |
| 149 | 100 |
| 150 | 94 |
| 151 | 81 |
| 152 | 105 |
| 153 | 93 |
| 154 | 89 |
| 155 | 97 |
| 156 | 97 |
| 157 | 106 |
| 158 | 96 |
| 159 | 100 |
| 160 | 102 |
| 161 | 99 |
| 162 | 106 |
| 163 | 87 |
| 164 | 116 |
| 165 | 108 |
| 166 | 96 |
| 168 | 108 |
| 169 | 110 |
| 170 | 95 |
| 171 | 104 |
| 172 | 94 |
| 173 | 114 |
| 174 | 105 |
| 176 | 95 |
| 177 | 94 |
| 178 | 90 |
| 179 | 102 |
| 180 | 101 |
| 181 | 103 |
| 182 | 92 |
| 183 | 94 |
| 184 | 104 |
| 185 | 86 |
| 186 | 105 |
| 187 | 102 |
| 188 | 104 |
| 189 | 95 |
| 190 | 101 |
| 191 | 101 |
| 192 | 104 |
| 193 | 105 |
| 194 | 103 |
| 195 | 110 |
| 196 | 98 |
| 197 | 100 |
| 198 | 112 |
| 199 | 105 |
| 200 | 104 |
| 201 | 102 |
| 202 | 93 |
| 203 | 107 |
| 204 | 111 |
| 205 | 105 |
| 206 | 95 |
| 207 | 98 |
| 208 | 103 |
| 209 | 97 |
| 210 | 95 |

### Example 19—Glucose Utilization in Cultured C2C12 Cells

The effect of the peptides on glucose homeostasis can be assessed using an assay to monitor glucose utilization by cultured cells such as mouse myotubes. Peptide arrays were initially prepared as 5 mM stock in DMSO and diluted to 0.5 mM in H2O; peptides were used at a final concentration of 5 μM (0.1% DMSO). Calculations for peptide concentrations based on approximate molecular weight of 2000 Daltons. C2C12 mouse muscle cell line was purchased from Millipore Sigma (Saint Louis, Mo.). C2C12 cells were seeded in standard medium (DMEM/low glucose (1 g/L)+10% Fetal Bovine Serum+antibiotics) at 7,500 cells/well on 96-well plates and grown to confluence at 37° C. in a humidified atmosphere of 5% CO2/95% air. Three days after plating the medium was removed and with Differentiation Medium (DMEM/low glucose (1 g/L)+2% Horse Serum+antibiotics) added. The cultures were placed at 37° C. in a humidified atmosphere of 5% CO2/95% air for 5 days with partially replacement of medium daily. After 5 days of differentiation, the medium was removed from myotube cultures and replaced with Assay Medium (DMEM/low glucose (1 g/L)+antibiotics). Culture were placed at 37° C. in a humidified atmosphere of 5% CO2/95% air for 5 h. Following the 5 h incubation the medium was removed and fresh Assay Medium containing compounds was added; 5 μM peptide or 1 mM metformin as a potent stimulator of glucose utilization. Cells were incubated for 22 h at 37° C. in a humidified atmosphere of 5% CO2/95% air. Following 22 h incubation, conditioned medium from each well was transferred to a fresh 96-well plate. Glucose concentrations in the medium were determined using a Glucose Assay kit (Abcam; Cambridge, Mass.) according to the manufacturer's instructions; absorbance measured using a Cytation 3 plate reader at 570 nm (BioTek, Winooski, Vt.). Untreated cells were used as reference for basal glucose utilization. Absorbance values were expressed as percent control activity relative to untreated treated cells for 5 μM peptide. The relative standard deviation from plate to plate of untreated control was <10%. Metformin at 1 mM dramatically increased glucose utilization, reducing extracellular glucose by 50-70% compared to untreated controls across all plates. Data presented are representative values from 2-3 independent experiments with each data point performed in triplicate in each independent experiment. The results are reported in Table 5.

TABLE 5
Glucose Utilization in Cultured C2C12 Cells

| | Percent of Control |
|---|---|
| SEQ ID NO: | Activity |
| 1 | 99 |
| 2 | 99 |
| 3 | 94 |
| 4 | 101 |
| 5 | 101 |
| 6 | 100 |
| 8 | 102 |
| 9 | 98 |
| 10 | 100 |
| 11 | 96 |
| 13 | 101 |
| 14 | 100 |
| 15 | 96 |
| 16 | 100 |
| 18 | 97 |
| 19 | 94 |
| 20 | 102 |
| 23 | 99 |
| 24 | 94 |
| 25 | 97 |
| 26 | 99 |
| 27 | 95 |
| 28 | 100 |
| 29 | 109 |
| 30 | 102 |
| 31 | 107 |
| 32 | 108 |
| 33 | 111 |
| 35 | 108 |
| 36 | 106 |
| 37 | 109 |
| 38 | 108 |
| 39 | 107 |
| 40 | 109 |
| 41 | 109 |
| 42 | 107 |
| 43 | 101 |
| 44 | 103 |
| 45 | 108 |
| 46 | 104 |
| 47 | 103 |
| 48 | 104 |
| 49 | 108 |
| 50 | 99 |
| 51 | 103 |
| 52 | 104 |
| 53 | 101 |
| 54 | 103 |
| 55 | 103 |
| 56 | 104 |
| 57 | 107 |
| 58 | 101 |
| 59 | 108 |
| 60 | 101 |
| 61 | 104 |
| 62 | 91 |
| 65 | 99 |
| 66 | 101 |
| 67 | 88 |
| 68 | 99 |
| 69 | 107 |
| 70 | 118 |
| 71 | 105 |
| 72 | 97 |
| 73 | 111 |
| 75 | 100 |
| 78 | 106 |
| 79 | 94 |
| 80 | 99 |
| 81 | 118 |
| 82 | 105 |
| 83 | 95 |
| 84 | 95 |
| 85 | 88 |
| 86 | 87 |
| 87 | 97 |
| 88 | 87 |
| 89 | 112 |
| 90 | 97 |
| 91 | 109 |
| 92 | 93 |
| 93 | 104 |
| 95 | 92 |
| 96 | 91 |
| 97 | 95 |
| 98 | 91 |
| 100 | 102 |
| 102 | 102 |
| 103 | 98 |
| 105 | 114 |
| 106 | 101 |
| 107 | 110 |
| 108 | 96 |
| 109 | 95 |
| 110 | 110 |
| 111 | 101 |
| 112 | 93 |
| 113 | 97 |
| 114 | 107 |
| 116 | 99 |
| 117 | 91 |
| 118 | 83 |
| 119 | 109 |
| 120 | 98 |
| 121 | 99 |
| 122 | 103 |
| 123 | 88 |
| 124 | 96 |
| 125 | 109 |
| 126 | 110 |
| 127 | 109 |
| 128 | 103 |
| 129 | 110 |
| 130 | 103 |
| 131 | 107 |
| 133 | 100 |
| 134 | 101 |
| 135 | 109 |
| 136 | 99 |
| 137 | 116 |
| 138 | 115 |
| 139 | 100 |
| 140 | 91 |
| 141 | 97 |
| 142 | 95 |
| 143 | 98 |
| 144 | 105 |
| 145 | 103 |
| 146 | 106 |
| 147 | 107 |
| 148 | 103 |
| 149 | 100 |
| 150 | 102 |
| 151 | 95 |
| 152 | 110 |
| 153 | 104 |
| 154 | 107 |
| 155 | 107 |
| 156 | 85 |
| 157 | 102 |
| 158 | 106 |
| 159 | 102 |
| 160 | 94 |
| 161 | 103 |
| 162 | 103 |
| 163 | 105 |
| 164 | 102 |
| 165 | 95 |
| 166 | 105 |
| 168 | 103 |
| 169 | 107 |
| 170 | 106 |
| 171 | 110 |
| 172 | 106 |
| 173 | 95 |
| 174 | 93 |
| 176 | 103 |
| 177 | 91 |
| 178 | 99 |
| 179 | 105 |
| 180 | 103 |
| 181 | 107 |
| 182 | 98 |
| 183 | 101 |
| 184 | 106 |
| 185 | 92 |
| 186 | 94 |
| 187 | 110 |
| 188 | 113 |
| 189 | 98 |
| 190 | 107 |
| 191 | 95 |
| 192 | 98 |
| 193 | 110 |
| 194 | 92 |
| 195 | 112 |
| 196 | 97 |
| 197 | 102 |
| 198 | 88 |
| 199 | 94 |
| 200 | 93 |
| 201 | 96 |
| 202 | 89 |
| 203 | 98 |
| 204 | 95 |
| 205 | 99 |
| 206 | 99 |
| 207 | 90 |
| 208 | 89 |
| 209 | 102 |
| 210 | 92 |

### Example 20—Glucose Production in Cultured H4-IIE Cells

The effect of the peptides on glucose homeostasis can be assessed using an assay to monitor glucose production by cultured cells such as rat hepatocytes. Peptide arrays were initially prepared as 5 mM stock in DMSO and diluted to 0.5 mM in H2O; peptides were used at a final concentration of 5 μM (0.1% DMSO). Calculations for peptide concentrations based on approximate molecular weight of 2000 Daltons. H4-IIE rat hepatic cell line was purchased from American Type Culture Collection (Manassas, Va.). H4-IIE cells were seeded in standard medium (DMEM/high glucose+10% Fetal Bovine Serum+antibiotics) at 100,000 cells/well on 96-well plates and allowed to adhere overnight at 37° C. in a humidified atmosphere of 5% CO2/95% air. 24 h after seeding, the medium was removed, cells were rinsed with glucose free DMEM, and the medium replaced with Glucose Production Medium (glucose free DMEM+2 mM sodium pyruvate+10 mM sodium lactate+antibiotics). The cultures were placed at 37° C. in a humidified atmosphere of 5% CO2/95% air overnight. The next morning, the medium was removed and fresh Glucose Production Medium containing compounds was added; 5 uM peptide or 0.5-1 nM insulin as a potent inhibitor of glucose production. Cells were incubated for 24 h at 37° C. in a humidified atmosphere of 5% CO2/95% air. Following 24 h incubation, conditioned medium from each well was transferred to a fresh 96-well plate. Glucose concentrations in the medium were determined using a Glucose Assay kit (Abcam; Cambridge, Mass.) according to the manufacturer's instructions; absorbance measured using a Cytation 3 plate reader at 570 nm (BioTek, Winooski, Vt.). Untreated cells were used as reference for maximal glucose production. Absorbance values were expressed as percent control activity relative to untreated treated cells for 5 μM peptide. The relative standard deviation from plate to plate of untreated control was <25%. Insulin was used as control to demonstrate inhibition of glucose production. Glucose levels for 0.5-1 nM insulin treatment were <5% of the untreated control value. Data are presented as representative values from 2-3 independent experiments with each data point performed in triplicate in each independent experiment. The results are shown in Table 6.

TABLE 6
Glucose Production in Cultured H4-IIE Cells

| | Percent of Control |
|---|---|
| SEQ ID NO: | Activity |
| 1 | 128 |
| 2 | 119 |
| 3 | 118 |
| 4 | 116 |
| 5 | 122 |
| 6 | 122 |
| 8 | 90 |
| 9 | 90 |
| 10 | 87 |
| 11 | 76 |
| 13 | 87 |
| 14 | 120 |
| 15 | 88 |
| 16 | 89 |
| 18 | 99 |
| 19 | 99 |
| 20 | 97 |
| 23 | 96 |
| 24 | 96 |
| 25 | 94 |
| 26 | 97 |
| 27 | 100 |
| 28 | 121 |
| 29 | 106 |
| 30 | 102 |
| 31 | 101 |
| 32 | 100 |
| 33 | 100 |
| 35 | 100 |
| 36 | 101 |
| 37 | 103 |
| 38 | 111 |
| 39 | 114 |
| 40 | 109 |
| 41 | 108 |
| 42 | 112 |
| 43 | 112 |
| 44 | 103 |
| 45 | 107 |
| 46 | 106 |
| 47 | 107 |
| 48 | 101 |
| 49 | 104 |
| 50 | 106 |
| 51 | 111 |
| 52 | 107 |
| 53 | 115 |
| 54 | 113 |
| 55 | 118 |
| 56 | 112 |
| 57 | 112 |
| 58 | 112 |
| 59 | 108 |
| 60 | 105 |
| 61 | 107 |
| 62 | 106 |
| 65 | 102 |
| 66 | 101 |
| 67 | 107 |
| 68 | 105 |
| 69 | 111 |
| 70 | 105 |
| 71 | 107 |
| 72 | 107 |
| 73 | 111 |
| 75 | 112 |
| 78 | 114 |
| 79 | 116 |
| 80 | 111 |
| 81 | 115 |
| 82 | 114 |
| 83 | 109 |
| 84 | 108 |
| 85 | 108 |
| 86 | 108 |
| 87 | 109 |
| 88 | 112 |
| 89 | 110 |
| 90 | 104 |
| 91 | 116 |
| 92 | 124 |
| 93 | 120 |
| 95 | 121 |
| 96 | 130 |
| 97 | 122 |
| 98 | 117 |
| 100 | 123 |
| 102 | 108 |
| 103 | 97 |
| 105 | 118 |
| 106 | 107 |
| 107 | 96 |
| 108 | 104 |
| 109 | 105 |
| 110 | 108 |
| 111 | 91 |
| 112 | 144 |
| 113 | 134 |
| 114 | 118 |
| 116 | 123 |
| 117 | 121 |
| 118 | 126 |
| 119 | 122 |
| 120 | 118 |
| 121 | 95 |
| 122 | 97 |
| 123 | 106 |
| 124 | 97 |
| 125 | 96 |
| 126 | 87 |
| 127 | 88 |
| 128 | 93 |
| 129 | 90 |
| 130 | 97 |
| 131 | 97 |
| 133 | 91 |
| 134 | 92 |
| 135 | 93 |
| 136 | 94 |
| 137 | 96 |
| 138 | 92 |
| 139 | 97 |
| 140 | 97 |
| 141 | 103 |
| 142 | 103 |
| 143 | 111 |
| 144 | 105 |
| 145 | 102 |
| 146 | 99 |
| 147 | 98 |
| 148 | 109 |
| 149 | 97 |
| 150 | 87 |
| 151 | 90 |
| 152 | 89 |
| 153 | 91 |
| 154 | 98 |
| 155 | 95 |
| 156 | 106 |
| 157 | 109 |
| 158 | 127 |
| 159 | 111 |
| 160 | 102 |
| 161 | 114 |
| 162 | 113 |
| 163 | 110 |
| 164 | 109 |
| 165 | 125 |
| 166 | 105 |
| 168 | 120 |
| 169 | 109 |
| 170 | 109 |
| 171 | 100 |
| 172 | 111 |
| 173 | 103 |
| 174 | 110 |
| 176 | 108 |
| 177 | 111 |
| 178 | 108 |
| 179 | 100 |
| 180 | 106 |
| 181 | 109 |
| 182 | 113 |
| 183 | 121 |
| 184 | 118 |
| 185 | 119 |
| 186 | 115 |
| 187 | 113 |
| 188 | 125 |
| 189 | 116 |
| 190 | 118 |
| 191 | 111 |
| 192 | 110 |
| 193 | 110 |
| 194 | 109 |
| 195 | 108 |
| 196 | 116 |
| 197 | 105 |
| 198 | 100 |
| 199 | 111 |
| 200 | 97 |
| 201 | 109 |
| 202 | 103 |
| 203 | 102 |
| 204 | 107 |
| 205 | 102 |
| 206 | 106 |
| 207 | 103 |
| 208 | 107 |
| 209 | 104 |
| 210 | 100 |

All of the articles and methods disclosed and claimed herein can be made and executed without undue experimentation in light of the present disclosure. While the articles and methods of this disclosure have been described in terms of preferred embodiments, it will be apparent to those of skill in the art that variations may be applied to the articles and methods without departing from the spirit and scope of the disclosure. All such variations and equivalents apparent to those skilled in the art, whether now existing or later developed, are deemed to be within the spirit and scope of the disclosure as defined by the appended claims. All patents, patent applications, and publications mentioned in the specification are indicative of the levels of those of ordinary skill in the art to which the disclosure pertains. The disclosure illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law). All headings and sub-headings are used herein for convenience only and should not be construed as being limiting in any way. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure. The citation and incorporation of patent documents herein is done for convenience only and does not reflect any view of the validity, patentability, and/or enforceability of such patent documents.

This disclosure includes all modifications and equivalents of the subject matter recited in the aspects appended hereto as permitted by applicable law.

The present application includes a Sequence Listing. To the extent differences exist between information/description of sequences in the specification and information in the Sequence Listing, the specification is controlling.

What is claimed:

1. A peptide comprising any of the following amino acid sequences:
SEQ ID NO: 1
MAAAPSGCPDPTS;
SEQ ID NO: 2
MAASVERGFIFLV;
SEQ ID NO: 3
MAGGFMLMIVVMKLMAPKMEETPA;
SEQ ID NO: 4
MAHTFPLNKTSR;
SEQ ID NO: 5
MAHYSQIPSRPHGWPPSD;
SEQ ID NO: 6
MAIAVVYPKTTIIPPK;
SEQ ID NO: 7
MAIMIINDEYWLVVLVMVHCPESMLLK;
SEQ ID NO: 8
MAPPLPPLEPTVKLT;
SEQ ID NO: 9
MAQTMPMYPNGSFFPE;
SEQ ID NO: 10
MATKKNFMEKGTRAGDMGSKPHS;
SEQ ID NO: 11
MAVVDGWVNTWVVPKSASPWKN;
SEQ ID NO: 12
MAYEGCCYSCKQEDNADVSGFWVEKWSVM;
SEQ ID NO: 13
MCSCVYVYSYCKYMVCSHDKP;
SEQ ID NO: 14
MDGCALLAVL;
SEQ ID NO: 15
MDMVPQGKDEKL;
SEQ ID NO: 16
MDSWVVGVNEWG;
SEQ ID NO: 17
MDWSNWVWGVQLYVWDFLGSGCWAWTLS;
SEQ ID NO: 18
MEAMGLAWNQLWGVRFLPFLS;
SEQ ID NO: 19
METDLDYSGLNSDHVGL;
SEQ ID NO: 20
MFDQENVVTSTESSPS;
SEQ ID NO: 21
MFEELINVWVWVYMSQWEFYDGPCNEQCY;
SEQ ID NO: 22
MFGSEFMYHSENSMMDHVTNNATGMNIMEK;
SEQ ID NO: 23
MFHLRYHNHRYPHRRQSI;
SEQ ID NO: 24
MFKLSFYFYVV;
SEQ ID NO: 25
MFLVGWWGMV;
SEQ ID NO: 26
MGACQGGSDESN;
SEQ ID NO: 27
MGCDDNVVDVVVT;
SEQ ID NO: 28
MGCSGSSVSQCYRVHTPQTKMPNAW;
SEQ ID NO: 29
MGDCAVCDA;
SEQ ID NO: 30
MGEGLEENPTNPITKPTLN;
SEQ ID NO: 31
MGEVGLLWLGRI;
SEQ ID NO: 32
MGGEWGMGVWTWGCFLVWM;
SEQ ID NO: 33
MGGGVLLGLQ;
SEQ ID NO: 34
MGGLGWGWPGGWVWGGGFMVEERWWEL;
SEQ ID NO: 35
MGGRWGLGWWRAG;
SEQ ID NO: 36
MGLAAVCVCWVGWAGVVLM;
SEQ ID NO: 37
MGLLEWLLCWHLLGRIINWWA;
SEQ ID NO: 38
MGLWLVCWVL;
SEQ ID NO: 39
MGNIIRAWCGEGCL;
SEQ ID NO: 40
MGSPPPAGSKKVVL;
SEQ ID NO: 41
MGSSRINPDPSPS;
SEQ ID NO: 42
MGSSRLAVLCPPLHGQVNFTG;
SEQ ID NO: 43
MGVGMEWGLLLRRGR;
SEQ ID NO: 44
MGVKFFTLST;
SEQ ID NO: 45
MHGELPWVVN;
SEQ ID NO: 46
MHHMKHPIIC;
SEQ ID NO: 47
MHKHYYNKHPHHYNLP;
SEQ ID NO: 48
MHKRLSPIYYSHRYLPDKRL;
SEQ ID NO: 49
MHLVFSSGGYARDSIA;
SEQ ID NO: 50
MHQSCKPEMKTFFQGQI;
SEQ ID NO: 51
MIEMGASFCHV;
SEQ ID NO: 52
MITVPAHKGMAMA;
SEQ ID NO: 53
MIYVSSDVGDWWKGGWGVWWVVHG;
SEQ ID NO: 54
MKFEIWLGWC;
SEQ ID NO: 55
MKHRQVLWVLSCGS;
SEQ ID NO: 56
MKNHRCISTT;
SEQ ID NO: 57
MKPFTVHSTLQSNPFSSPWMTPL;
SEQ ID NO: 58
MKPMSPMRLY;
SEQ ID NO: 59
MKSPSTLTTQSKTPSAYFSSFSP;
SEQ ID NO: 60
MLEIVMGMETYHMSNA;
SEQ ID NO: 61
MLFMRGNAMSGAPII;
SEQ ID NO: 62
MLGVVGWCRL;
SEQ ID NO: 63
MLGWVVGSFFMGGVWVGRSGIGGMLFEFM;
SEQ ID NO: 64
MLHNNPNPNTNYLPNWKQNTQMGLSL;
SEQ ID NO: 65
MLKPETSSDSPSA;
SEQ ID NO: 66
MLLGWRFQLGAWVGGLQ;
SEQ ID NO: 67
MLLMWWVSEPHCVVVNM;
SEQ ID NO: 68
MLLNSHRCSMSKDNHHSP;
SEQ ID NO: 69
MLMVEWWLGWLHM;
SEQ ID NO: 70
MLNWSNNLTNGTSYP;
SEQ ID NO: 71
MLPLHIYHNTMGLTHPPH;
SEQ ID NO: 72
MLQGQAHLSILFSI;
SEQ ID NO: 73
MLRSMLWNLWSKPQFHAHRP;
SEQ ID NO: 74
MLSPKPQQLNQQNCSPEHYEPQLKTQ;
SEQ ID NO: 75
MLTPGLVNFVPATAVTRLTQVN;
SEQ ID NO: 76
MLTSKYSNQPSTITHQLQLQSHPSPT;
SEQ ID NO: 77
MLVPFDRGDLEGELTGTGMLACVILL;
SEQ ID NO: 78
MMAHLGKKPVSGG;
SEQ ID NO: 79
MMCLRFRWCGLWS;
SEQ ID NO: 80
MMDPEHMNSMALKKAWVQMC;
SEQ ID NO: 81
MMFPIRLHNSPIRP;
SEQ ID NO: 82
MMGITMKKIITNAWAVTMTL;
SEQ ID NO: 83
MMGMWLVGHYVLSC;
SEQ ID NO: 84
MMKGKMKWKVKNRV;
SEQ ID NO: 85
MMKLRLTPWRLPDPPNHH;
SEQ ID NO: 86
MMLSWDDIIYGG;
SEQ ID NO: 87
MMMFVYSAMKN;
SEQ ID NO: 88
MMMTIECLHSHFPH;
SEQ ID NO: 89
MMNSDSIIPS;
SEQ ID NO: 90
MMSVWKVAVQTFNCYYYVLQALIN;
SEQ ID NO: 91
MMTHPTTPLTINTKPP;
SEQ ID NO: 92
MMTWSPWKPVATKNVEP;
SEQ ID NO: 93
MMWKTIVVFQLQEHQWPQYAKLTP;
SEQ ID NO: 94
MNCGGCLWGLVGSGYGVSSGVCVLG;
SEQ ID NO: 95
MNHIMAKGHDG;
SEQ ID NO: 96
MNHNQYYQSMLIINNHNSYSNKT;
SEQ ID NO: 97
MNNSSSTVQP;
SEQ ID NO: 98
MNPDQPHHLLLSLYTAIFSKPWW;
SEQ ID NO: 99
MPFWVEVMMEVEIWCCEIVLGNSFSSQV;
SEQ ID NO: 100
MPIIRRMSWSP;
SEQ ID NO: 101
MPIVTIMSPSWLEVEKATIFLMSFCV;
SEQ ID NO: 102
MPLRLEWVQWGVGGWPWVCC;
SEQ ID NO: 103
MPPKDKIWNLV;
SEQ ID NO: 104
MPPLCKIHCRIHLYYQSLPHNNIHVP;
SEQ ID NO: 105
MPRKCWLYPSRTN;
SEQ ID NO: 106
MPTNLPTLNST;
SEQ ID NO: 107
MPTPILIFRSPWSLYSYPT;
SEQ ID NO: 108
MQFTLATPKCYGPGA;
SEQ ID NO: 109
MQNPPHSSPHSSPLPRYSYLSPLLY;
SEQ ID NO: 110
MQPYRRYRFHPRLSMIYPTLQLM;
SEQ ID NO: 111
MQRWFFMSLVVVVVRA;
SEQ ID NO: 112
MQSGVLQSLAVTEIKYCNLL;
SEQ ID NO: 113
MQTKSLQSPQ;
SEQ ID NO: 114
MQWAILGLFVVG;
SEQ ID NO: 115
MRLLAWGNTWWQLLWNEGLFFWLELE;
SEQ ID NO: 116
MSGVRKEVSLL;
SEQ ID NO: 117
MSKKIPATTMVLEWS;
SEQ ID NO: 118
MSLSYLQDSTY;
SEQ ID NO: 119
MSLTPHILPTMP;
SEQ ID NO: 120
MSMPVLGWQWG;
SEQ ID NO: 121
MSNMKTFSSA;
SEQ ID NO: 122
MSNNMAFSTFKG;
SEQ ID NO: 123
MSRNHSFCLNYMPIRFSLILFVVTH;
SEQ ID NO: 124
MSSAWIIWFRLFSI;
SEQ ID NO: 125
MSSDEFANTMPV;
SEQ ID NO: 126
MSTHLPNYHTNLSYR;
SEQ ID NO: 127
MSTINQQVIITLTVNPTQACS;
SEQ ID NO: 128
MSWSNIEVVNPIVDMDS;
SEQ ID NO: 129
MSYRAHTPKMLVMPFPY;
SEQ ID NO: 130
MTAPVKLQGVWM;
SEQ ID NO: 131
MTELLTPNWTNLSPY;
SEQ ID NO: 132
MTKNFHQTPPPPLLATALKHISAKPQKQ;
SEQ ID NO: 133
MTLGHNMIYLHTS;
SEQ ID NO: 134
MTNIFGVWAR;
SEQ ID NO: 135
MTNPENQNERKSVRFIHCPHNP;
SEQ ID NO: 136
MTNTLNSIHPPLP;
SEQ ID NO: 137
MTSLHNSFYSKDTSLRTPLMTP;
SEQ ID NO: 138
MTSPKIQNNNTPDHTANNQY;
SEQ ID NO: 139
MTWPTEQVTLGMTAQSYS;
SEQ ID NO: 140
MTYSPEQSQLQYMHQQTMFNQ;
SEQ ID NO: 141
MVEGWLLYLLVSMG;
SEQ ID NO: 142
MVEVGYNVVRVV;
SEQ ID NO: 143
MVGYLIPVWVLAIVCSDMLKPLS;
SEQ ID NO: 144
MVIICFEVSLISHCWVVISRLLM;
SEQ ID NO: 145
MVKPHLQNASI;
SEQ ID NO: 146
MVLEFGLVGYFLLGGGSGWV;
SEQ ID NO: 147
MVLFLLGMVG;
SEQ ID NO: 148
MVLNLRVHRLRRTNLQLLHTSPIIP;
SEQ ID NO: 149
MVLSSPPIFRMSCSLL;
SEQ ID NO: 150
MVLWWFGGSG;
SEQ ID NO: 151
MVNGRRTSVGV;
SEQ ID NO: 152
MVRMLLVQWW;
SEQ ID NO: 153
MVSMMTSMGM;
SEQ ID NO: 154
MVSSGDWYSWCE;
SEQ ID NO: 155
MVVCMVITFIWSCTKIFGA;
SEQ ID NO: 156
MVVHWMSGVGLPWLWGVGV;
SEQ ID NO: 157
MVVKGPLSEGGHPWGREGIWL;
SEQ ID NO: 158
MWEEAGRMSEGCLG;
SEQ ID NO: 159
MWGVSDMLVFLEV;
SEQ ID NO: 160
MWLGVGLGWVG;
SEQ ID NO: 161
MWLIEEYAMSDF;
SEQ ID NO: 162
MWLVLWLWML;
SEQ ID NO: 163
MWMPPTLPHIR;
SEQ ID NO: 164
MWNDLLQCSEP;
SEQ ID NO: 165
MWRVLGALWW;
SEQ ID NO: 166
MWSHPSHHSTINITNKWLL;
SEQ ID NO: 167
MWVIEEYAKILRSWVWFNPPQLPAMMDKIE;
SEQ ID NO: 168
MYCNWDCSGE;
SEQ ID NO: 169
MYLNGTCSAS;
SEQ ID NO: 170
MYNYAKAPTL;
SEQ ID NO: 171
MYRHLQQTLMKATK;
SEQ ID NO: 172
MYTNKQCSTSNYY;
SEQ ID NO: 173
MYTPTNNVQPVTTTNQRP;
SEQ ID NO: 174
MYYSDGYWGVSWGMGV;
SEQ ID NO: 175
MYEMHSGCWWVSQYLGGTQICFPMKEQ;
SEQ ID NO: 176
MKNIEAPLAW;
SEQ ID NO: 177
MRMGASIGSTTRLSTS;
SEQ ID NO: 178
MVQTVSISWASEMLVLVSFVVSV;
SEQ ID NO: 179
MSSSMMGEVASC;
SEQ ID NO: 180
MVVGSWCWSLSWVVGMR;
SEQ ID NO: 181
MCHWAGGASNTGDA;
SEQ ID NO: 182
MFAVSMMWSLE;
SEQ ID NO: 183
MMKVDATMDFT;
SEQ ID NO: 184
MASAKGCSSP;
SEQ ID NO: 185
MLVMLEVMFLVN;
SEQ ID NO: 186
MIINDEYWLVVLVMVHCPESMLLK;
SEQ ID NO: 187
AYEGCCYSCKQEDNADVSGFWVEK;
SEQ ID NO: 188
DWSNWVWGVQLYVWDFLGSGCWAW;
SEQ ID NO: 189
FEELINVWVWVYMSQWEFYDGPCN;
SEQ ID NO: 190
MYHSENSMMDHVTNNATGMNIMEK;
SEQ ID NO: 191
LGWGWPGGWVWGGGFMVEERWWEL;
SEQ ID NO: 192
LGWVVGSFFMGGVWVGRSGIGGML;
SEQ ID NO: 193
LHNNPNPNTNYLPNWKQNTQMGLS;
SEQ ID NO: 194
LSPKPQQLNQQNCSPEHYEPQLKT;
SEQ ID NO: 195
LTSKYSNQPSTITHQLQLQSHPSP;
SEQ ID NO: 196
LVPFDRGDLEGELTGTGMLACVIL;
SEQ ID NO: 197
WVEVMMEVEIWCCEIVLGNSFSSQ;
SEQ ID NO: 198
PIVTIMSPSWLEVEKATIFLMSFC;
SEQ ID NO: 199
PLCKIHCRIHLYYQSLPHNNIHVP;
SEQ ID NO: 200
RLLAWGNTWWQLLWNEGLFFWLEL;
SEQ ID NO: 201
TKNFHQTPPPPLLATALKHISAKP;
SEQ ID NO: 202
MWVIEEYAKILRSWVWFNPPQLPA;
SEQ ID NO: 203
KNIEAPLAW;
SEQ ID NO: 204
RMGASIGSTTRLSTS;
SEQ ID NO: 205
VQTVSISWASEMLVLVSFVVSV;
SEQ ID NO: 206
SSSMMGEVASC;
SEQ ID NO: 207
VVGSWCWSLSWVVGMR;
SEQ ID NO: 208
CHWAGGASNTGDA;
SEQ ID NO: 209
FAVSMMWSLE;
and
SEQ ID NO: 210
MKVDATMDFT;
or pharmaceutically acceptable salts thereof.

2. An isolated peptide comprising an amino acid sequence having at least about 80% sequence identity, as measured by BLAST, with a peptide according to claim 1.

3. An isolated peptide comprising an amino acid sequence having at least about 90% sequence identity, as measured by BLAST, with a peptide according to claim 1.

4. A peptide comprising a peptide selected from
SEQ ID NO: 1
MAAAPSGCPDPTS;
SEQ ID NO: 2
MAASVERGFIFLV;
SEQ ID NO: 3
MAGGFMLMIVVMKLMAPKMEETPA;
SEQ ID NO: 4
MAHTFPLNKTSR;
SEQ ID NO: 5
MAHYSQIPSRPHGWPPSD;
SEQ ID NO: 6
MAIAVVYPKTTIIPPK;
SEQ ID NO: 7
MAIMIINDEYWLVVLVMVHCPESMLLK;
SEQ ID NO: 8
MAPPLPPLEPTVKLT;
SEQ ID NO: 9
MAQTMPMYPNGSFFPE;
SEQ ID NO: 10
MATKKNFMEKGTRAGDMGSKPHS;
SEQ ID NO: 11
MAVVDGWVNTWVVPKSASPWKN;
SEQ ID NO: 12
MAYEGCCYSCKQEDNADVSGFWVEKWSVM;
SEQ ID NO: 13
MCSCVYVYSYCKYMVCSHDKP;
SEQ ID NO: 14
MDGCALLAVL;
SEQ ID NO: 15
MDMVPQGKDEKL;
SEQ ID NO: 16
MDSWVVGVNEWG;
SEQ ID NO: 17
MDWSNWVWGVQLYVWDFLGSGCWAWTLS;
SEQ ID NO: 18
MEAMGLAWNQLWGVRFLPFLS;
SEQ ID NO: 19
METDLDYSGLNSDHVGL;
SEQ ID NO: 20
MFDQENVVTSTESSPS;
SEQ ID NO: 21
MFEELINVWVWVYMSQWEFYDGPCNEQCY;
SEQ ID NO: 22
MFGSEFMYHSENSMMDHVTNNATGMNIMEK;
SEQ ID NO: 23
MFHLRYHNHRYPHRRQSI;
SEQ ID NO: 24
MFKLSFYFYVV;
SEQ ID NO: 25
MFLVGWWGMV;
SEQ ID NO: 26
MGACQGGSDESN;
SEQ ID NO: 27
MGCDDNVVDVVVT;
SEQ ID NO: 28
MGCSGSSVSQCYRVHTPQTKMPNAW;
SEQ ID NO: 29
MGDCAVCDA;
SEQ ID NO: 30
MGEGLEENPTNPITKPTLN;
SEQ ID NO: 31
MGEVGLLWLGRI;
SEQ ID NO: 32
MGGEWGMGVWTWGCFLVWM;
SEQ ID NO: 33
MGGGVLLGLQ;
SEQ ID NO: 34
MGGLGWGWPGGWVWGGGFMVEERWWEL;
SEQ ID NO: 35
MGGRWGLGWWRAG;
SEQ ID NO: 36
MGLAAVCVCWVGWAGVVLM;
SEQ ID NO: 37
MGLLEWLLCWHLLGRIINWWA;
SEQ ID NO: 38
MGLWLVCWVL;
SEQ ID NO: 39
MGNIIRAWCGEGCL;
SEQ ID NO: 40
MGSPPPAGSKKVVL;
SEQ ID NO: 41
MGSSRINPDPSPS;
SEQ ID NO: 42
MGSSRLAVLCPPLHGQVNFTG;
SEQ ID NO: 43
MGVGMEWGLLLRRGR;
SEQ ID NO: 44
MGVKFFTLST;
SEQ ID NO: 45
MHGELPWVVN;
SEQ ID NO: 46
MHHMKHPIIC;
SEQ ID NO: 47
MHKHYYNKHPHHYNLP;
SEQ ID NO: 48
MHKRLSPIYYSHRYLPDKRL;
SEQ ID NO: 49
MHLVFSSGGYARDSIA;
SEQ ID NO: 50
MHQSCKPEMKTFFQGQI;
SEQ ID NO: 51
MIEMGASFCHV;
SEQ ID NO: 52
MITVPAHKGMAMA;
SEQ ID NO: 53
MIYVSSDVGDWWKGGWGVWWVVHG;
SEQ ID NO: 54
MKFEIWLGWC;
SEQ ID NO: 55
MKHRQVLWVLSCGS;
SEQ ID NO: 56
MKNHRCISTT;
SEQ ID NO: 57
MKPFTVHSTLQSNPFSSPWMTPL;
SEQ ID NO: 58
MKPMSPMRLY;
SEQ ID NO: 59
MKSPSTLTTQSKTPSAYFSSFSP;
SEQ ID NO: 60
MLEIVMGMETYHMSNA;
SEQ ID NO: 61
MLFMRGNAMSGAPII;
SEQ ID NO: 62
MLGVVGWCRL;
SEQ ID NO: 63
MLGWVVGSFFMGGVWVGRSGIGGMLFEFM;
SEQ ID NO: 64
MLHNNPNPNTNYLPNWKQNTQMGLSL;
SEQ ID NO: 65
MLKPETSSDSPSA;
SEQ ID NO: 66
MLLGWRFQLGAWVGGLQ;
SEQ ID NO: 67
MLLMWWVSEPHCVVVNM;
SEQ ID NO: 68
MLLNSHRCSMSKDNHHSP;
SEQ ID NO: 69
MLMVEWWLGWLHM;
SEQ ID NO: 70
MLNWSNNLTNGTSYP;
SEQ ID NO: 71
MLPLHIYHNTMGLTHPPH;
SEQ ID NO: 72
MLQGQAHLSILFSI;
SEQ ID NO: 73
MLRSMLWNLWSKPQFHAHRP;
SEQ ID NO: 74
MLSPKPQQLNQQNCSPEHYEPQLKTQ;
SEQ ID NO: 75
MLTPGLVNFVPATAVTRLTQVN;
SEQ ID NO: 76
MLTSKYSNQPSTITHQLQLQSHPSPT;
SEQ ID NO: 77
MLVPFDRGDLEGELTGTGMLACVILL;
SEQ ID NO: 78
MMAHLGKKPVSGG;
SEQ ID NO: 79
MMCLRFRWCGLWS;
SEQ ID NO: 80
MMDPEHMNSMALKKAWVQMC;
SEQ ID NO: 81
MMFPIRLHNSPIRP;
SEQ ID NO: 82
MMGITMKKIITNAWAVTMTL;
SEQ ID NO: 83
MMGMWLVGHYVLSC;
SEQ ID NO: 84
MMKGKMKWKVKNRV;
SEQ ID NO: 85
MMKLRLTPWRLPDPPNHH;
SEQ ID NO: 86
MMLSWDDIIYGG;
SEQ ID NO: 87
MMMFVYSAMKN;
SEQ ID NO: 88
MMMTIECLHSHFPH;
SEQ ID NO: 89
MMNSDSIIPS;
SEQ ID NO: 90
MMSVWKVAVQTFNCYYYVLQALIN;
SEQ ID NO: 91
MMTHPTTPLTINTKPP;
SEQ ID NO: 92
MMTWSPWKPVATKNVEP;
SEQ ID NO: 93
MMWKTIVVFQLQEHQWPQYAKLTP;
SEQ ID NO: 94
MNCGGCLWGLVGSGYGVSSGVCVLG;
SEQ ID NO: 95
MNHIMAKGHDG;
SEQ ID NO: 96
MNHNQYYQSMLIINNHNSYSNKT;
SEQ ID NO: 97
MNNSSSTVQP;
SEQ ID NO: 98
MNPDQPHHLLLSLYTAIFSKPWW;
SEQ ID NO: 99
MPFWVEVMMEVEIWCCEIVLGNSFSSQV;
SEQ ID NO: 100
MPIIRRMSWSP;
SEQ ID NO: 101
MPIVTIMSPSWLEVEKATIFLMSFCV;
SEQ ID NO: 102
MPLRLEWVQWGVGGWPWVCC;
SEQ ID NO: 103
MPPKDKIWNLV;
SEQ ID NO: 104
MPPLCKIHCRIHLYYQSLPHNNIHVP;
SEQ ID NO: 105
MPRKCWLYPSRTN;
SEQ ID NO: 106
MPTNLPTLNST;
SEQ ID NO: 107
MPTPILIFRSPWSLYSYPT;
SEQ ID NO: 108
MQFTLATPKCYGPGA;
SEQ ID NO: 109
MQNPPHSSPHSSPLPRYSYLSPLLY;
SEQ ID NO: 110
MQPYRRYRFHPRLSMIYPTLQLM;
SEQ ID NO: 111
MQRWFFMSLVVVVVRA;
SEQ ID NO: 112
MQSGVLQSLAVTEIKYCNLL;
SEQ ID NO: 113
MQTKSLQSPQ;
SEQ ID NO: 114
MQWAILGLFVVG;
SEQ ID NO: 115
MRLLAWGNTWWQLLWNEGLFFWLELE;
SEQ ID NO: 116
MSGVRKEVSLL;
SEQ ID NO: 117
MSKKIPATTMVLEWS;
SEQ ID NO: 118
MSLSYLQDSTY;
SEQ ID NO: 119
MSLTPHILPTMP;
SEQ ID NO: 120
MSMPVLGWQWG;
SEQ ID NO: 121
MSNMKTFSSA;
SEQ ID NO: 122
MSNNMAFSTFKG;
SEQ ID NO: 123
MSRNHSFCLNYMPIRFSLILFVVTH;
SEQ ID NO: 124
MSSAWIIWFRLFSI;
SEQ ID NO: 125
MSSDEFANTMPV;
SEQ ID NO: 126
MSTHLPNYHTNLSYR;
SEQ ID NO: 127
MSTINQQVIITLTVNPTQACS;
SEQ ID NO: 128
MSWSNIEVVNPIVDMDS;
SEQ ID NO: 129
MSYRAHTPKMLVMPFPY;
SEQ ID NO: 130
MTAPVKLQGVWM;
SEQ ID NO: 131
MTELLTPNWTNLSPY;
SEQ ID NO: 132
MTKNFHQTPPPPLLATALKHISAKPQKQ;
SEQ ID NO: 133
MTLGHNMIYLHTS;
SEQ ID NO: 134
MTNIFGVWAR;
SEQ ID NO: 135
MTNPENQNERKSVRFIHCPHNP;
SEQ ID NO: 136
MTNTLNSIHPPLP;
SEQ ID NO: 137
MTSLHNSFYSKDTSLRTPLMTP;
SEQ ID NO: 138
MTSPKIQNNNTPDHTANNQY;
SEQ ID NO: 139
MTWPTEQVTLGMTAQSYS;
SEQ ID NO: 140
MTYSPEQSQLQYMHQQTMFNQ;
SEQ ID NO: 141
MVEGWLLYLLVSMG;
SEQ ID NO: 142
MVEVGYNVVRVV;
SEQ ID NO: 143
MVGYLIPVWVLAIVCSDMLKPLS;
SEQ ID NO: 144
MVIICFEVSLISHCWVVISRLLM;
SEQ ID NO: 145
MVKPHLQNASI;
SEQ ID NO: 146
MVLEFGLVGYFLLGGGSGWV;
SEQ ID NO: 147
MVLFLLGMVG;
SEQ ID NO: 148
MVLNLRVHRLRRTNLQLLHTSPIIP;
SEQ ID NO: 149
MVLSSPPIFRMSCSLL;
SEQ ID NO: 150
MVLWWFGGSG;
SEQ ID NO: 151
MVNGRRTSVGV;
SEQ ID NO: 152
MVRMLLVQWW;
SEQ ID NO: 153
MVSMMTSMGM;
SEQ ID NO: 154
MVSSGDWYSWCE;
SEQ ID NO: 155
MVVCMVITFIWSCTKIFGA;
SEQ ID NO: 156
MVVHWMSGVGLPWLWGVGV;
SEQ ID NO: 157
MVVKGPLSEGGHPWGREGIWL;
SEQ ID NO: 158
MWEEAGRMSEGCLG;
SEQ ID NO: 159
MWGVSDMLVFLEV;
SEQ ID NO: 160
MWLGVGLGWVG;
SEQ ID NO: 161
MWLIEEYAMSDF;
SEQ ID NO: 162
MWLVLWLWML;
SEQ ID NO: 163
MWMPPTLPHIR;
SEQ ID NO: 164
MWNDLLQCSEP;
SEQ ID NO: 165
MWRVLGALWW;
SEQ ID NO: 166
MWSHPSHHSTINITNKWLL;
SEQ ID NO: 167
MWVIEEYAKILRSWVWFNPPQLPAMMDKIE;
SEQ ID NO: 168
MYCNWDCSGE;
SEQ ID NO: 169
MYLNGTCSAS;
SEQ ID NO: 170
MYNYAKAPTL;
SEQ ID NO: 171
MYRHLQQTLMKATK;
SEQ ID NO: 172
MYTNKQCSTSNYY;
SEQ ID NO: 173
MYTPTNNVQPVTTTNQRP;
SEQ ID NO: 174
MYYSDGYWGVSWGMGV;
SEQ ID NO: 175
MYEMHSGCWWVSQYLGGTQICFPMKEQ;
SEQ ID NO: 176
MKNIEAPLAW;
SEQ ID NO: 177
MRMGASIGSTTRLSTS;
SEQ ID NO: 178
MVQTVSISWASEMLVLVSFVVSV;
SEQ ID NO: 179
MSSSMMGEVASC;
SEQ ID NO: 180
MVVGSWCWSLSWVVGMR;
SEQ ID NO: 181
MCHWAGGASNTGDA;
SEQ ID NO: 182
MFAVSMMWSLE;
SEQ ID NO: 183
MMKVDATMDFT;
SEQ ID NO: 184
MASAKGCSSP;
SEQ ID NO: 185
MLVMLEVMFLVN;
SEQ ID NO: 186
MIINDEYWLVVLVMVHCPESMLLK;
SEQ ID NO: 187
AYEGCCYSCKQEDNADVSGFWVEK;
SEQ ID NO: 188
DWSNWVWGVQLYVWDFLGSGCWAW;
SEQ ID NO: 189
FEELINVWVWVYMSQWEFYDGPCN;
SEQ ID NO: 190
MYHSENSMMDHVTNNATGMNIMEK;
SEQ ID NO: 191
LGWGWPGGWVWGGGFMVEERWWEL;
SEQ ID NO: 192
LGWVVGSFFMGGVWVGRSGIGGML;
SEQ ID NO: 193
LHNNPNPNTNYLPNWKQNTQMGLS;
SEQ ID NO: 194
LSPKPQQLNQQNCSPEHYEPQLKT;
SEQ ID NO: 195
LTSKYSNQPSTITHQLQLQSHPSP;
SEQ ID NO: 196
LVPFDRGDLEGELTGTGMLACVIL;
SEQ ID NO: 197
WVEVMMEVEIWCCEIVLGNSFSSQ;
SEQ ID NO: 198
PIVTIMSPSWLEVEKATIFLMSFC;
SEQ ID NO: 199
PLCKIHCRIHLYYQSLPHNNIHVP;
SEQ ID NO: 200
RLLAWGNTWWQLLWNEGLFFWLEL;
SEQ ID NO: 201
TKNFHQTPPPPLLATALKHISAKP;
SEQ ID NO: 202
MWVIEEYAKILRSWVWFNPPQLPA;
SEQ ID NO: 203
KNIEAPLAW;
SEQ ID NO: 204
RMGASIGSTTRLSTS;
SEQ ID NO: 205
VQTVSISWASEMLVLVSFVVSV;
SEQ ID NO: 206
SSSMMGEVASC;
SEQ ID NO: 207
VVGSWCWSLSWVVGMR;
SEQ ID NO: 208
CHWAGGASNTGDA;
SEQ ID NO: 209
FAVSMMWSLE;
and
SEQ ID NO: 210
MKVDATMDFT;
wherein the amino acid sequence of the peptide is modified such that it comprises a deletion, insertion or substitution of one to four amino acids.

5. A peptide comprising any of the following amino acid sequences:
SEQ ID NO: 12
MAYEGCCYSCKQEDNADVSGFWVEKWSVM;
SEQ ID NO: 21
MFEELINVWVWVYMSQWEFYDGPCNEQCY;
SEQ ID NO: 22
MFGSEFMYHSENSMMDHVTNNATGMNIMEK;
SEQ ID NO: 36
MGLAAVCVCWVGWAGVVLM;
SEQ ID NO: 51
MIEMGASFCHV;
SEQ ID NO: 59
MKSPSTLTTQSKTPSAYFSSFSP;
SEQ ID NO: 64
MLHNNPNPNTNYLPNWKQNTQMGLSL;
SEQ ID NO: 67
MLLMWWVSEPHCVVVNM;
SEQ ID NO: 74
MLSPKPQQLNQQNCSPEHYEPQLKTQ;
SEQ ID NO: 76
MLTSKYSNQPSTITHQLQLQSHPSPT;
SEQ ID NO: 77
MLVPFDRGDLEGELTGTGMLACVILL;
SEQ ID NO: 94
MNCGGCLWGLVGSGYGVSSGVCVLG;
SEQ ID NO: 99
MPFWVEVMMEVEIWCCEIVLGNSFSSQV;
SEQ ID NO: 101
MPIVTIMSPSWLEVEKATIFLMSFCV;
SEQ ID NO: 104
MPPLCKIHCRIHLYYQSLPHNNIHVP;
SEQ ID NO: 109
MQNPPHSSPHSSPLPRYSYLSPLLY;
SEQ ID NO: 111
MQRWFFMSLVVVVVRA;
SEQ ID NO: 112
MQSGVLQSLAVTEIKYCNLL;
SEQ ID NO: 115
MRLLAWGNTWWQLLWNEGLFFWLELE;
SEQ ID NO: 122
MSNNMAFSTFKG;
SEQ ID NO: 127
MSTINQQVIITLTVNPTQACS;
SEQ ID NO: 131
MTELLTPNWTNLSPY;
SEQ ID NO: 137
MTSLHNSFYSKDTSLRTPLMTP;
SEQ ID NO: 141
MVEGWLLYLLVSMG;
SEQ ID NO: 144
MVIICFEVSLISHCWVVISRLLM;
SEQ ID NO: 148
MVLNLRVHRLRRTNLQLLHTSPIIP;
SEQ ID NO: 167
MWVIEEYAKILRSWVWFNPPQLPAMMDKIE;
and
SEQ ID NO: 171
MYRHLQQTLMKATK;
or a pharmaceutically acceptable salt thereof.

6. A peptide of any one of claims 1-5, wherein the peptide comprises substitution with at least one amino acid selected from (i) an amino acid having a D-configuration, and (ii) a non-naturally occurring amino acid residue; or pharmaceutically acceptable salts thereof.

7. A peptide of any one of claims 1-6, further comprising a duration enhancing moiety, optionally further comprising a metabolically cleavable linker coupling the peptide to the duration enhancing moiety.

8. A conjugate comprising the peptide of any one of claims 1-7 conjugated to a heterologous moiety.

9. A composition comprising a peptide of any of claims 1-7 or a conjugate of claim 8 and a pharmaceutically acceptable excipient.

10. The composition of claim 9, wherein the pharmaceutically acceptable excipient is not found in nature.

11. A method of modulating cell viability comprising administering a peptide of any one of claims 1-7, a conjugate of claim 8, or a composition of claim 9 or 10.

12. A method of treating cell proliferation in patient in need of such treatment, comprising administering to the patient a pharmacologically effective amount of a peptide of any one of claims 1-7, a conjugate of claim 8, or a composition of claim 9 or 10.

13. A method of treating an apoptosic disease in a patient in need of such treatment, comprising administering to the patient a pharmacologically effect amount of a peptide of any one of claims 1-7, a conjugate of claim 8, or a composition of claim 9 or 10.

14. A method of treating a metabolic disease in a patient in need of such treatment, comprising administering to the patient a pharmacologically effect amount of a peptide of any one of claims 1-7, a conjugate of claim 8, or a composition of claim 9 or 10.

15. A method of providing cytoprotection in a patient in need of such treatment, comprising administering to the patient a pharmacologically effect amount of a peptide of any one claims 1-7, a conjugate of claim 8, or a composition of claim 9 or 10.

16. A nucleic acid comprising a nucleotide sequence that encodes a peptide according to any one of claims 1-7.

17. A vector, preferably an expression vector, comprising the nucleic acid according to claim 15.

18. A host cell, preferably an isolated host cell, comprising the nucleic acid of claim 16 or the vector of claim 17.

19. A method of treating a metabolic disease in a subject in need thereof, comprising administering to the subject a peptide or peptide analog, a conjugate, or a composition of any one of claims 1-10, in an amount effective to treat the metabolic disease.

20. The method of claim 19, wherein the disease is selected from the group consisting of obesity, diabetes (e.g., Type 2 diabetes), cognitive disorders and/or neurodegenerative disorders, cardiovascular disease, fatty liver disease, and gastrointestinal disease.

21. A method of treating a cancer in a subject in need thereof, comprising administering to the subject a peptide or peptide analog, a conjugate, or a composition of any one of claims 1-10, in an amount effective to treat the cancer.

22. The method of claim 21, wherein the cancer is lung cancer, pancreatic cancer, breast cancer, prostate cancer, ovarian cancer, or hepatocellular cancer.

23. A method of treating a liver disease in a subject in need thereof, comprising administering to the subject a peptide or peptide analog, a conjugate, or a composition of any one of claims 1-10, in an amount effective to treat the liver disease.

24. The method of claim 23, wherein the liver disease is a fatty liver disease.

25. The method of claim 24, wherein the fatty liver disease is NAFLD or NASH.

26. A method of modulating fatty acid metabolism in a subject in need thereof, comprising administering to the subject a peptide or peptide analog, a conjugate, or a composition of any one of claims 1-10, in an amount effective to modulate fatty acid metabolism.

27. The method of claim 26, wherein fatty acid metabolism is increased in the subject after the a peptide or peptide analog, a conjugate, or a composition is administered to the subject.

28. A method of reducing body weight in a subject in need thereof, comprising administering to the subject a peptide or peptide analog, a conjugate, or a composition of any one of claims 1-10, in an amount effective to reduce body weight in the subject.

29. The peptides, peptide analogs, conjugates, compositions of any one of claims 1-10 for use in a therapeutic treatment of a metabolic disease, cancer, liver disease, or any disease, disorder, or medical condition described herein.

30. Use of any one of the peptides, peptide analogs, conjugates, compositions of any one of claims 1-10 in therapeutic treatment of a metabolic disease, cancer, liver disease, or any disease, disorder, or medical condition described herein.

31. Use of any one of the peptides, peptide analogs, conjugates, or compositions of any one claims 1-18 in the manufacture of a medicament for treating a metabolic disease, cancer, liver disease, or any disease, disorder, or medical condition described herein.
